SEQUENCE LISTING (27 sequences)

SEQ ID NO: 1 - DNA, 1271 bp, Homo sapiens
atgctcataactgttaatgaaagcagattcaaagcaacaccaccaccactgaagtatttttagttatataagattggaactaccaagcatgtggctcctggtcagtgtaattctaatctcacggatatcctctgttgggggagaagcaacattttgtgattttccaaaaataaaccatggaattctatatgatgaagaaaaatataagccattttcccaggttcctacaggggaagttttctattactcctgtgaatataattttgtgtctccttcaaaatcattttggactcgcataacatgcacagaagaaggatggtcaccaacaccaaagtgtctcagactgtgtttctttccttttgtggaaaatggtcattctgaatcttcaggacaaacacatctggaaggtgatactgtgcaaattatttgcaacacaggatacagacttcaaaacaatgagaacaacatttcatgtgtagaacggggctggtccacccctcccaaatgcaggtccactgacacttcctgtgtgaatccgcccacagtacaaaatgctcatatactgtcgagacagatgagtaaatatccatctggtgagagagtacgttatgaatgtaggagcccttatgaaatgtttggggatgaagaagtgatgtgtttaaatggaaactggacagaaccacctcaatgcaaagattctacgggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttatttgagaacaggtgaatcagctgaatttgtgtgtaaacggggatatcgtctttcatcacgttctcacacattgcgaacaacatgttgggatgggaaactggagtatccaacttgtgcaaaaagatagaatcaatcataaaatgcacacctttattcagaactttagtattaaatcagttcttaatttcatttttaagtattgttttactcctttttattcatacgtaaaattttggattaatttgtgaaaatgtaattataagctgagaccggtggctctcttcttaaaagcaccatattaaaacttggaaaactaa

SEQ ID NO: 2 - protein, 330 aa, Homo sapiens
MWLLVSVILISRISSVGGEATFCDFPKINHGILYDEEKYKPFSQVPTGEVFYYSCEYNFVSPSKSFWTRITCTEEGWSPTPKCLRLCFFPFVENGHSESSGQTHLEGDTVQIICNTGYRLQNNENNISCVERGWSTPPKCRSTDTSCVNPPTVQNAHILSRQMSKYPSGERVRYECRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYLRTGESAEFVCKRGYRLSSRSHTLRTTCWDGKLEYPTCAKR

SEQ ID NO: 3 - DNA, 1062 bp, Homo sapiens
cagttagtacactgaaattcaaagtcatgctcataactgttaatgaaagcagattcaaagcaacaccaccaccactgaagtatttttagttatataagattggaactaccaagcatgtggctcctggtcagtgtaattctaatctcacggatatcctctgttgggggagaagcaatgttctgtgattttccaaaaataaaccatggaattctatatgatgaagaaaaatataagccattttcccaagttcctacaggggaagttttctattactcctgtgaatataattttgtgtctccttcaaaatccttttggactcgcataacgtgcgcagaagaaggatggtcaccaacaccaaagtgtctcagactgtgtttctttccttttgtggaaaatggtcattctgaatcttcaggacaaacacatctggaaggtgatactgtacaaattatttgcaacacaggatacagacttcaaaacaatgagaacaacatttcatgtgtagaacggggctggtccactcctcccaaatgcaggtccactatttctgcagaaaaatgtgggccccctccacctattgacaatggagacattacttcattcctgttgtcagtatatgctccaggttcatcagttgagtaccagtgccagaacttgtatcaacttgagggtaacaatcaaataacatgtagaaacggacaatggtcagaaccaccaaaatgcttagatccatgtgtaatatcacaagaaattatggaaaaatataacataaaattaaagtggacaaaccaacaaaagctttattcaagaacaggtgacatagttgaatttgtttgtaaatctggatatcatccaacaaaatctcattcatttcgagcaatgtgtcagaatgggaaactggtatatcccagttgtgaagaaaaatagaatcaatggcattactattagtaaaatgcacacctttttctgaatttactattatatttgttttcaatttcatttttcaagtactgttttactcatttttattcataaataaagttttgtgttgatttgtgaaaa

SEQ ID NO: 4 - protein, 270 aa, Homo sapiens
MWLLVSVILISRISSVGGEAMFCDFPKINHGILYDEEKYKPFSQVPTGEVFYYSCEYNFVSPSKSFWTRITCAEEGWSPTPKCLRLCFFPFVENGHSESSGQTHLEGDTVQIICNTGYRLQNNENNISCVERGWSTPPKCRSTISAEKCGPPPPIDNGDITSFLLSVYAPGSSVEYQCQNLYQLEGNNQITCRNGQWSEPPKCLDPCVISQEIMEKYNIKLKWTNQQKLYSRTGDIVEFVCKSGYHPTKSHSFRAMCQNGKLVYPSCEEK

SEQ ID NO: 5 - DNA, 1645 bp, Homo sapiens
gaaccacacttggtaactaataatgaaagatttcaaaccccaaacagtgcaactgaaacttttgtattagcatactactgagaatatctaacatgttgttactaatcaatgtcattctgaccttgtgggtttcctgtgctaatggacaagtgaaaccttgtgattttccagacattaaacatggaggtctatttcatgagaatatgcgtagaccatactttccagtagctgtaggaaaatattactcctattactgtgatgaacattttgagactccttcaggaagttactgggattacattcattgcacacaaaatgggtggtcaccagcagtaccatgtctcagaaaatgttattttccttatttggaaaatggatataatcaaaattatggaagaaagtttgtacagggtaactctacagaagttgcctgccatcctggctacggtcttccaaaagcgcagaccacagttacatgtacggagaaaggctggtctcctactcccagatgcatccgtgtcagaacatgctcaaaatcagatatagaaattgaaaatggattcatttccgaatcttcctctatttatattttaaataaagaaatacaatataaatgtaaaccaggatatgcaacagcagatggaaattcttcaggatcaattacatgtttgcaaaatggatggtcagcacaaccaatttgcattaattcttcagaaaagtgtgggcctcctccacctattagcaatggtgataccacctcctttctactaaaagtgtatgtgccacagtcaagagtcgagtaccaatgccagccctactatgaacttcagggttctaattatgtaacatgtagtaatggagagtggtcggaaccaccaagatgcatacatccatgtataataactgaagaaaacatgaataaaaataacataaagttaaaaggaagaagtgacagaaaatattatgcaaaaacaggggataccattgaatttatgtgtaaattgggatataatgcaaatacatcaattctatcatttcaagcagtgtgtcgggaagggatagtggaataccccagatgcgaataaggcagcattgttaccctaaatgtatgtccaacttccacttttccacttctcactcttatggtctcaaagcttgcaaagatagcttctgatattgttgtaatttctactttatttcaaagaaaattaatataatagtttcaatttgcaacttaatatattctcaaaaatatattaaaacaaactaaattattgcttatgcttgtactaaaataataaaaactactcttatattggacttcttatcaatgaattagtaagtatagagacagacagctgaatggctttctgcatattgtatagtatacctagacatagaaacaaaatgactttagattttatttggggaagtaataataccataaaattagatattaaaattgtaagtgaagataaacacactatagtattcccttattgtagccatggtcctctagatgcagttaaccaaatagggtcatttttattaaaagtagtgtttcctggcaaacactgacattacatcattatcatgatttaaaggaaatagtactagagaaggtgaattattatcattttcctgt

SEQ ID NO: 6 - protein, 330 aa, Homo sapiens
MLLLINVILTLWVSCANGQVKPCDFPDIKHGGLFHENMRRPYFPVAVGKYYSYYCDEHFETPSGSYWDYIHCTQNGWSPAVPCLRKCYFPYLENGYNQNYGRKFVQGNSTEVACHPGYGLPKAQTTVTCTEKGWSPTPRCIRVRTCSKSDIEIENGFISESSSIYILNKEIQYKCKPGYATADGNSSGSITCLQNGWSAQPICINSSEKCGPPPPISNGDTTSFLLKVYVPQSRVEYQCQPYYELQGSNYVTCSNGEWSEPPRCIHPCIITEENMNKNNIKLKGRSDRKYYAKTGDTIEFMCKLGYNANTSILSFQAVCREGIVEYPRCE

SEQ ID NO: 7 - DNA, 1292 bp, Homo sapiens
tgaaagatttcaaaccccaaacagtgcaactgaaacttttgcattactatactactgagaatatctaacatgttgttactaatcaatgtcattctgaccttgtgggtttcctgtgctaatggacaagcaatgaaaccttgtgagtttccagaaattcaacatggacatctatattatgagaatacgcgtagaccatactttccagtagctacaggacaatcttactcctattactgtgaccaaaattttgtgactccttcaggaagttactgggattacattcactgcacacaagatgggtggttgccaacagtcccatgcctcagaacatgctcaaaatcagatatagaaattgaaaatggattcatttctgaatcttcctctatttatattttaaataaagaaatacaatataaatgtaaaccaggatatgcaacagcagatggaaattcttcaggttcaattacatgtttgcaaaatggatggtcagcacaaccaatttgcattaaattttgtgatatgcctgtttttgagaattccagagccaagagtaatggcatgcggtttaagctccatgacacattggactacgaatgctacgatggatatgaaatcagttatggaaacaccacaggttccatagtgtgtggtgaagatgggtggtcccatttcccaacatgttataattcttcagaaaagtgtgggcctcctccacctattagcaatggtgataccacctcctttctactaaaagtgtatgtgccacagtcaagagtcgagtaccaatgccagtcctactatgaacttcagggttctaattatgtaacatgtagtaatggagagtggtcggaaccaccaagatgcatacatccatgtataataactgaagaaaacatgaataaaaataacatacagttaaaaggaaaaagtgacataaaatattatgcaaaaacaggggataccattgaatttatgtgtaaattgggatataatgcgaatacatcagttctatcatttcaagcagtgtgtagggaaggcatagtggaataccccagatgcgaataaggcagcattgttaccctaaatgtatgtccaacttccacttctcactcttatggtctcaaagcttgcaaagatagcttctgatattgttgtaatttctactttatttcaaagaaaattaatataatagtttcaatttgcaacttaatatgttctcaaaaatatgttaaaacaaactaaattattgcttatgcttgtactaaaataataaaaactacccttatattgga

SEQ ID NO: 8 - protein, 577 aa, Homo sapiens
MLLLINVILTLWVSCANGQVKPCDFPEIQHGGLYYKSLRRLYFPAAAGQSYSYYCDQNFVTPSGSYWDYIHCTQDGWSPTVPCLRTCSKSDVEIENGFISESSSIYILNEETQYNCKPGYATAEGNSSGSITCLQNGWSTQPICIKFCDMPVFENSRAKSNGMWFKLHDTLDYECYDGYESSYGNTTDSIVCGEDGWSHLPTCYNSSENCGPPPPISNGDTTSFPQKVYLPWSRVEYQCQSYYELQGSKYVTCSNGDWSEPPRCISMKPCEFPEIQHGHLYYENTRRPYFPVATGQSYSYYCDQNFVTPSGSYWDYIHCTQDGWLPTVPCLRTCSKSDIEIENGFISESSSIYILNKEIQYKCKPGYATADGNSSGSITCLQNGWSAQPICIKFCDMPVFENSRAKSNGMRFKLHDTLDYECYDGYEISYGNTTGSIVCGEDGWSHFPTCYNSSEKCGPPPPISNGDTTSFLLKVYVPQSRVEYQCQSYYELQGSNYVTCSNGEWSEPPRCIHPCIITEENMNKNNIQLKGKSDIKYYAKTGDTIEFMCKLGYNANTSVLSFQAVCREGIVEYPRCE

SEQ ID NO: 9 - protein, 331 aa, Homo sapiens
MLLLINVILTLWVSCANGQEVKPCDFPEIQHGGLYYKSLRRLYFPAAAGQSYSYYCDQNFVTPSGSYWDYIHCTQDGWSPTVPCLRTCSKSDIEIENGFISESSSIYILNKEIQYKCKPGYATADGNSSGSITCLQNGWSAQPICIKFCDMPVFENSRAKSNGMRFKLHDTLDYECYDGYEISYGNTTGSIVCGEDGWSHFPTCYNSSEKCGPPPPISNGDTTSFLLKVYVPQSRVEYQCQSYYELQGSNYVTCSNGEWSEPPRCIHPCIITEENMNKNNIQLKGKSDIKYYAKTGDTIEFMCKLGYNANTSVLSFQAVCREGIVEYPRCE

SEQ ID NO: 10 - DNA, 2810 bp, Homo sapiens
agtacattgaaattcaaagtcatgcttgtaactgttaatgaaagcagatttaaagcaacaccaccatcactggagtatttttagttatatacgattgagactaccaagcatgttgctcttattcagtgtaatcctaatctcatgggtatccactgttgggggagaaggaacactttgtgattttccaaaaatacaccatggatttctgtatgatgaagaagattataaccctttttcccaagttcctacaggggaagttttctattactcctgtgaatataattttgtgtctccttcaaaatccttttggactcgcataacatgcacagaagaaggatggtcaccaacaccgaagtgtctcagaatgtgttcctttccttttgtgaaaaatggtcattctgaatcttcaggactaatacatctggaaggtgatactgtacaaattatttgcaacacaggatacagccttcaaaacaatgagaaaaacatttcgtgtgtagaacggggctggtccactcctcccatatgcagcttcactaaaggagaatgtcatgttccaattttagaagccaatgtagatgctcagccaaaaaaagaaagctacaaagttggagacgtgttgaaattctcctgcagaaaaaatcttataagagttggatcagactcagttcaatgttaccaatttgggtggtcacctaactttccaacatgcaaaggacaagtacgatcatgtggtccacctcctcaactctccaatggtgaagttaaggagataagaaaagaggaatatggacacaatgaagtagtggaatatgattgcaatcctaattttataataaacgggcctaagaaaatacaatgtgtggatggagaatggacaactttacccacttgtgttgaacaagtgaaaacatgtggatacatacctgaactcgagtacggttatgttcagccgtctgtccctccctatcaacatggagtttcagtcgaggtgaattgcagaaatgaatatgcaatgattggaaataacatgattacctgtattaatggaatatggacagagcttcctatgtgtgttgcaacacaccaacttaagaggtgcaaaatagcaggagttaatataaaaacattactcaagctatctgggaaagaatttaatcataattctagaatacgttacagatgttcagacatcttcagatacaggcactcagtctgtataaacgggaaatggaatcctgaagtagactgcacagaaaaaagggaacaattctgcccaccgccacctcagatacctaatgctcagaatatgacaaccacagtgaattatcaggatggagaaaaagtagctgttctctgtaaagaaaactatctacttccagaagcaaaagaaattgtatgtaaagatggacgatggcaatcattaccacgctgtgttgagtctactgcatattgtgggccccctccatctattaacaatggagataccacctcattcccattatcagtatatcctccagggtcaacagtgacgtaccgttgccagtccttctataaactccagggctctgtaactgtaacatgcagaaataaacagtggtcagaaccaccaagatgcctagatccatgtgtggtatctgaagaaaacatgaacaaaaataacatacagttaaaatggagaaacgatggaaaactctatgcaaaaacaggggatgctgttgaattccagtgtaaattcccacataaagcgatgatatcatcaccaccatttcgagcaatctgtcaggaagggaaatttgaatatcctatatgtgaatgaagcaagcataattttcctgaatatattcttcaaacatccatctatgctaaaagtagccattatgtagccaattctgtagttacttcttttattctttcaggtgttgtttaactcagttttatttagaactctggatttttagagctttagaaatttgtaagctgagagaacaatgtttcacttaataggagggtgtcttagtccatattacattgttataacagagtatcacagactggataacttctaaccaatagtttatttgtttcataaatctaaaagctgagaagtccaagatggtggggctgcctctggtgagggtcttctcgaagcatcataatatgctggaaggcatcacaacatggtggaagggatcacgtggcaaaagagcatgtacatgggagtgagagaaaaagagagagagagacagagtggcgggggcggggaggagcgcaaactcatcctttataaagacaccactcctgagataacaatccaatcccatgataatgacattaatccattcaagaagatagagctctcgtgacttaatcaccttctaaagatctcacctgacaacactgttgcattggcagttaagtttccacgtaaactttcggggacacattcaaaccacaggagaaactcaaattgttcctgggcaaatcacaacatggggaattttattcataaatgtccacagaaacagtaaatgttctcgcttcagtacttaattcatctaatccctcctgtttgtctcaaattataggataactttgaaactttctgaattaacgttatttaaaaggaaatgtagatgttattttagtctctatcttcatgttattatcacttaaaaacctgcgaaagctgtcaacttttgtggttgtagcaagtattaataaatatttataaatcctctaatgtaagtctagctacctatccaatactaaataccccttaaagtattaaatgcactatctgctgtaaa

SEQ ID NO: 11 - protein, 569 aa, Homo sapiens
MLLLFSVILISWVSTVGGEGTLCDFPKIHHGFLYDEEDYNPFSQVPTGEVFYYSCEYNFVSPSKSFWTRITCTEEGWSPTPKCLRMCSFPFVKNGHSESSGLIHLEGDTVQIICNTGYSLQNNEKNISCVERGWSTPPICSFTKGECHVPILEANVDAQPKKESYKVGDVLKFSCRKNLIRVGSDSVQCYQFGWSPNFPTCKGQVRSCGPPPQLSNGEVKEIRKEEYGHNEVVEYDCNPNFIINGPKKIQCVDGEWTTLPTCVEQVKTCGYIPELEYGYVQPSVPPYQHGVSVEVNCRNEYAMIGNNMITCINGIWTELPMCVATHQLKRCKIAGVNIKTLLKLSGKEFNHNSRIRYRCSDIFRYRHSVCINGKWNPEVDCTEKREQFCPPPPQIPNAQNMTTTVNYQDGEKVAVLCKENYLLPEAKEIVCKDGRWQSLPRCVESTAYCGPPPSINNGDTTSFPLSVYPPGSTVTYRCQSFYKLQGSVTVTCRNKQWSEPPRCLDPCVVSEENMNKNNIQLKWRNDGKLYAKTGDAVEFQCKFPHKAMISSPPFRAICQEGKFEYPICE

SEQ ID NO: 12 - protein, 32 aa, Homo sapiens
PFSQVPTGEVFYYSCEYNFVSPSKSFWTRITC

SEQ ID NO: 13 - protein, 7 aa, Homo sapiens
YYSCEYN

SEQ ID NO: 14 - DNA, 50 bp, Homo sapiens
gctgacaaggatgatctcgagaaaagagaagcaacattttgtgattttcc

SEQ ID NO: 15 - DNA, 35 bp, Homo sapiens
gccgcccatggacatctaagtggacctgcatttgg

SEQ ID NO: 16 - DNA, 44 bp, Homo sapiens
gagatataccatgggcacttcctgtgtgaatccgcccacagtac

SEQ ID NO: 17 - DNA, 46 bp, Homo sapiens
gccggatcctctatctttttgcacaagttggatactccagtttccc

SEQ ID NO: 18 - DNA, 47 bp, Homo sapiens
tataccatgggcgaaaaatgtgggccccctccacctattgacaatgg

SEQ ID NO: 19 - DNA, 47 bp, Homo sapiens
cgtgccggatcctatttttcttcacaactgggatataccagtttccc

SEQ ID NO: 20 - DNA, 67 bp, Homo sapiens
caagttcctacaggggaagttttctcttactactgtgaagagaattttgtgtctccttcaaaatcct

SEQ ID NO: 21 - DNA, 67 bp, Homo sapiens
aggattttgaaggagacacaaaattctcttcacagtagtaagagaaaacttcccctgtaggaacttg

SEQ ID NO: 22 - protein, 122 aa, Homo sapiens
TFCDFPKINHGILYDEEKYKPFSQVPTGEVFYYSCEYNFVSPSKSFWTRITCTEEGWSPTPKCLRLCFFPFVENGHSESSGQTHLEGDTVQIICNTGYRLQNNENNISCVERGWSTPPKCRS

SEQ ID NO: 23 - protein, 122 aa, Homo sapiens
MFCDFPKINHGILYDEEKYKPFSQVPTGEVFYYSCEYNFVSPSKSFWTRITCAEEGWSPTPKCLRLCFFPFVENGHSESSGQTHLEGDTVQIICNTGYRLQNNENNISCVERGWSTPPKCRS

SEQ ID NO: 24 - protein, 122 aa, Homo sapiens
TLCDFPKIHHGFLYDEEDYNPFSQVPTGEVFYYSCEYNFVSPSKSFWTRITCTEEGWSPTPKCLRMCSFPFVKNGHSESSGLIHLEGDTVQIICNTGYSLQNNEKNISCVERGWSTPPICSF

SEQ ID NO: 25 - protein, 62 aa, Homo sapiens
RTCSKSDIEIENGFISESSSIYILNKEIQYKCKPGYATADGNSSGSITCLQNGWSAQPICIN

SEQ ID NO: 26 - protein, 62 aa, Homo sapiens
RTCSKSDIEIENGFISESSSIYILNKEIQYKCKPGYATADGNSSGSITCLQNGWSAQPICIK

SEQ ID NO: 27 - protein, 61 aa, Homo sapiens
RTCSKSDIEIENGFISESSSIYILNKEIQYKCKPGYATADGNSSGSITCLQNGWSAQPICI

(19) United States
(12) Patent Application Publication
(10) Pub. No.: US 2015/0361183 A1
(43) Pub. Date: Dec. 17, 2015

(54) INHIBITION OF THE COMPLEMENT SYSTEM

(71) Applicant: IMPERIAL INNOVATIONS PLC, London (GB)

(72) Inventors: Matthew Pickering, London (GB); Susan Lea, Oxford (GB); Elena Goicoechea de Jorge, London (GB)

(73) Assignee: Imperial Innovations Limited, London (GB)

(21) Appl. No.: 14/764,920

(22) PCT Filed: Jan. 30, 2014
(86) PCT No.: PCT/GB2014/050258

§ 371 Date: Jul. 30, 2015

(30) Foreign Application Priority Data
Jan. 30, 2013 (GB) 13011632.4

(51) Int. Cl. C07K 16/40; G01N 33/58; G01N 33/68; C12N 15/113

(52) U.S. Cl. CPC C07K 16/40; C12N 15/113; G01N 33/582; G01N 33/6845; C12N 2310/14; C12N 2310/11; G01N 2500/02; G01N 2333/4716; C07K 2317/34; C07K 2317/76

(57) ABSTRACT

Agents and compounds which can be used to modulate the activity of the complement system, novel biological targets associated with such modulation, and pharmaceutical compositions, medicaments and methods of treatment for use in preventing, ameliorating or treating diseases that are characterised by inappropriate complement activity. These diseases include age-related macular degeneration (AMD), meningitis, renal disease, autoimmune disease and inflammation. Therapeutic antibodies and screening assays for identifying agents useful in treating these diseases are also provided.

The present invention relates to the complement system, and in particular to agents and compounds which can be used to modulate, and particularly negatively modulate, the activity of the complement system. The invention provides novel biological targets associated with such modulation, and also pharmaceutical compositions, medicaments and methods of treatment for use in preventing, ameliorating or treating diseases that are characterised by inappropriate complement activity, for example age-related macular degeneration (AMD), meningitis, renal disease, autoimmune disease or inflammation. The invention also extends to therapeutic antibodies, and to screening assays for identifying agents useful in treating these diseases.

The complement system is a key component of innate immunity and host defence. Regulation of complement activation is of major importance to enable activation on pathogens whilst preventing activation on healthy host tissue. Complement factor H (CFH) is an abundant plasma protein whose major function is to down-regulate C3 activation through the alternative pathway and C3b amplification loops. Complete CFH deficiency is associated with severe secondary C3 deficiency due to uncontrolled consumption through these pathways. CFH mutations increase susceptibility to the renal diseases, atypical haemolytic uraemic syndrome (aHUS) and dense deposit disease (DDD), whilst polymorphic variation of CFH has been strongly associated with important human diseases, including age-related macular degeneration (AMD) and meningococcal sepsis (Clin Exp Immunol 151(2):210-230; Immunobiology 217(11):1034-1046). It is now evident that variation in the complement factor H-related (CFHR) genes is also important in disease susceptibility and a role for these CFHR proteins in pathology has been unequivocally demonstrated by diseases associated with both mutations and polymorphisms in the CFHR genes.

The five CFHR proteins (CFHR1-5), together with CFH, comprise a family of structurally related proteins. CFH is a well-characterized negative regulator of complement C3 activation, but the biological roles of the CFHR proteins are poorly understood. The frequent finding among healthy individuals of an allele lacking both CFHR3 and CFHR1 genes (ΔCFHR3-1) (Ann Med 38 (8):592-604), and, less commonly, an allele lacking both CFHR1 and CFHR4 (Blood 114(19):4261-4271), demonstrated that these proteins were biologically non-essential. However, genetic variation across the CFHR locus influences susceptibility to disease: the ΔCFHR3-1 deletion copy number variation (CNV) polymorphism confers protection against IgA nephropathy (Nat Genet 43(4)321-327) and age-related macular degeneration (AMD) (Nat Genet 38(10):1173-1177), and susceptibility to systemic lupus erythematosus (PLoS Genet 7(5):e1002079). Two rare CNV polymorphisms within the CFHR locus are associated with familial C3 glomerulopathy. Among individuals with Cypriot ancestry the disease segregated with an internal duplication affecting the CFHR5 gene whilst in an Irish family the disease was associated with a heterozygous hybrid CFHR3-1 gene that was present on an allele that contained intact copies of both the CFHR3 and CFHR1 genes.

In view of the above, it will be appreciated that there are many disease conditions that are associated with inappropriate complement activation, and particularly excessive complement activity. Thus, it is an aim of embodiments of the present invention to provide novel targets involved in the complement system, as well as improved therapeutics, which can be used to modulate complement activation in order to treat these diseases.

The inventors set out to achieve this by focusing their studies on the structure and mechanism of the CFHR1-5 proteins and/or Complement factor H (CFH), and their effects on complement activation, particularly on their ability to bind to C3 fragments, such as C3b. As a result of their research, they now have a detailed understanding of how these proteins interact with each other, and have demonstrated how manipulating the concentration of certain proteins or using agents capable of blocking protein interactions can be used in therapy to treat disorders caused by excessive complement activation.

Thus, in a first aspect of the invention, there is provided an agent, which:—

(i) reduces the concentration or activity of at least one complement factor H-related (CFHR) protein selected from a group consisting of: CFHR1, CFHR2, CFHR3, CFHR4 and CFHR5; or
(ii) reduces or inhibits dimerisation or higher order assembly of at least one CFHR protein selected from a group consisting of: CFHR1, CFHR2, CFHR3, CFHR4 and CFHR5, for use in diagnosis or therapy.

The agents of the first aspect may therefore be used as a medicament. Preferably, agents of the invention may be used to treat any disease which is characterised by excessive complement activation, for example renal disease, age-related macular degeneration (AMD), meningitis, autoimmune disease or inflammation etc.

Therefore, in a second aspect, there is provided an agent, which:—

(i) reduces the concentration or activity of at least one complement factor H-related (CFHR) protein selected from a group consisting of: CFHR1, CFHR2, CFHR3, CFHR4 and CFHR5; or
(ii) reduces or inhibits dimerisation or higher order assembly of at least one CFHR protein selected from a group consisting of: CFHR1, CFHR2, CFHR3, CFHR4 and CFHR5, for use in the treatment, prevention or amelioration of a disease characterised by excessive complement activation.

In a third aspect, there is provided a method of treating, preventing or ameliorating a disease characterised by excessive complement activation in a subject, the method comprising administering, to a subject in need of such treatment, a therapeutically effective amount of an agent, which:—

(i) reduces the concentration or activity of at least one complement factor H-related (CFHR) protein selected from a group consisting of: CFHR1, CFHR2, CFHR3, CFHR4 and CFHR5; or
(ii) reduces or inhibits dimerisation or higher order assembly of at least one CFHR protein selected from a group consisting of: CFHR1, CFHR2, CFHR3, CFHR4 and CFHR5, to treat, prevent or ameliorate a disease characterised by excessive complement activation in the subject.

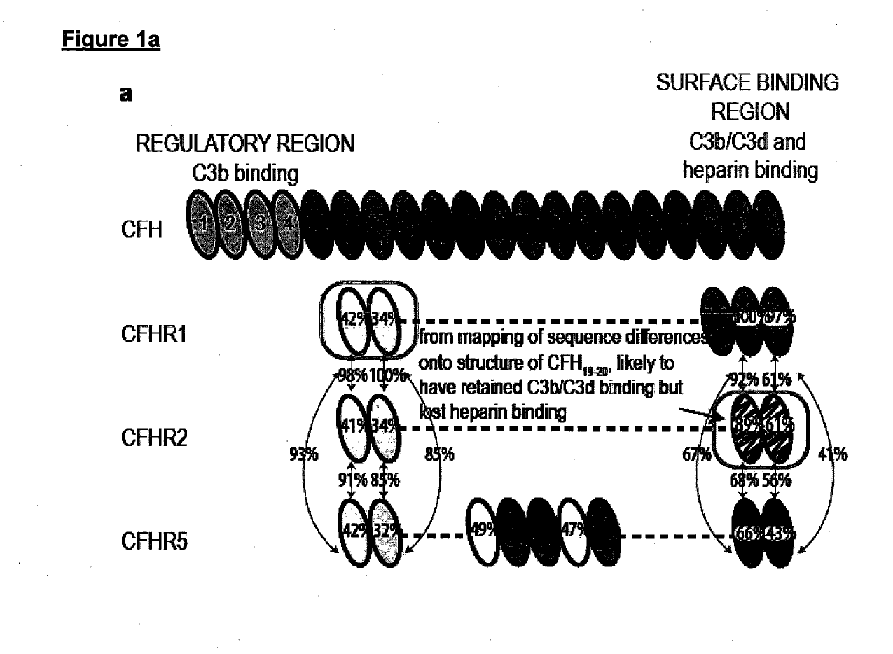

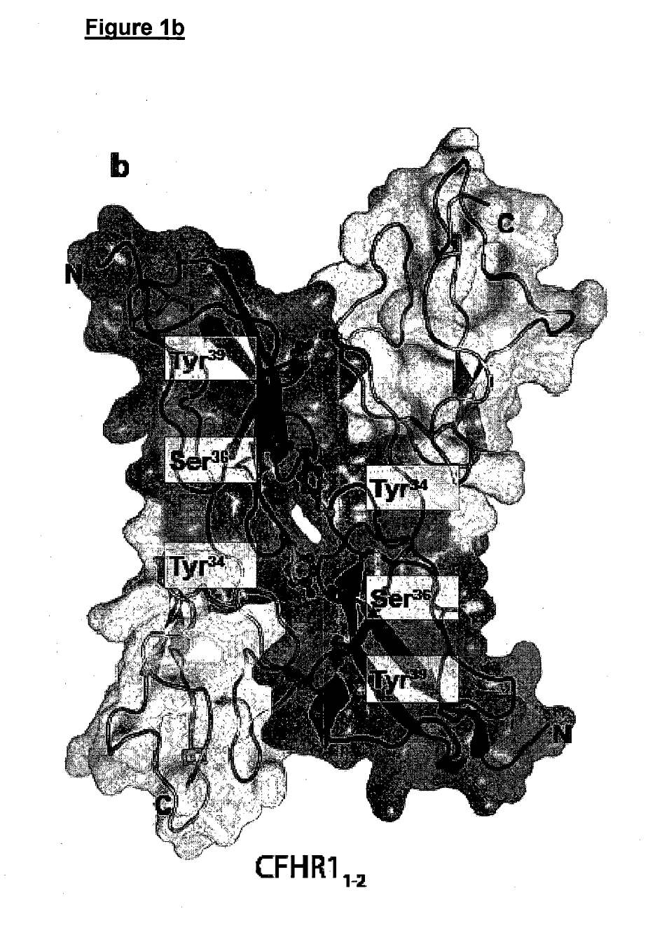

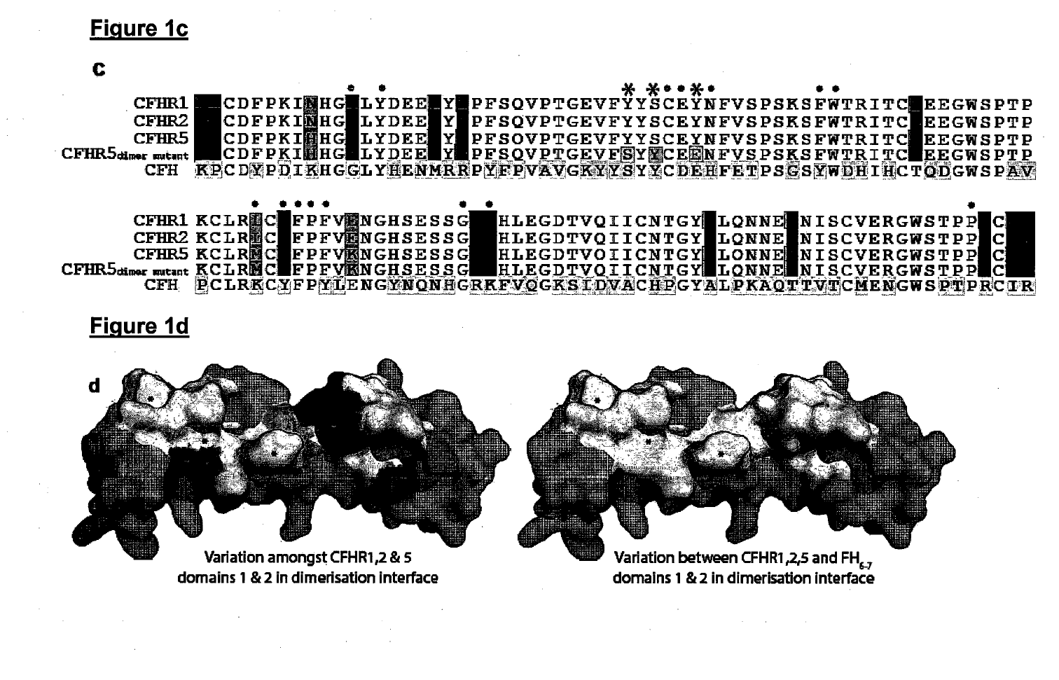

As described in the Examples, and as shown in FIG. 1c, the inventors were surprised to observe that CFHR1, CFHR2 and CFHR5 contain a shared dimerisation motif that resides within their common two amino-terminal domains. This dimerisation motif enables the formation of three homodimers (i.e. CFHR1-CFHR1, CFHR2-CFHR2 and CFHR5-CFHR5) and three heterodimers (CFHR1-CFHR2, CFHR1-CFHR5 and CFHR2-CFHR5). Furthermore, they found that, in the presence of the ΔCFHR3-1 deletion polymorphism, the absence of CFHR1 reduces the potential combinations to two homodimers (CFHR2-CFHR2 and CFHR5-CFHR5) and a single heterodimer, CFHR2-CFHR5.

The term “higher order assembly” can mean trimerisation or tetramerisation, or greater. The inventors have demonstrated that it is this formation of dimers, trimers or tetramers (homo- and hetero), which significantly enhances the avidity of these proteins in vivo for ligand (e.g. the C3b protein in the complement pathway), and that this property enables these proteins to surprisingly out-compete CFH at physiologically relevant concentrations. This dimerisation-driven avidity enables these proteins to function as de-regulators of the complement system by acting as competitive antagonists of CFH. The data described herein demonstrate that qualitative and quantitative variation within the CFHR family provides a novel means by which complement activation can be modulated in vivo.

Up until now, the notion that the CFHRs can bind bivalently as dimers to molecules of C3b, iC3b, C3dg and C3d, and surface polyanions and surface carbohydrate moieties, was not known, and has enabled the inventors to more clearly understand that diseases that are characterised by inappropriate complement activation can be treated by reducing the concentration or activity of CFHR1-5, or by preventing dimerisation or higher order assembly of these proteins, rather than by increasing the concentration or activity of CFHR1-5, as currently taught by the prior art. Advantageously, the agents of the invention are effective in treating disease because, in some embodiments, they can target the common dimerisation domain in the CFHR's, and neutralise (i.e. deactivate) and clear the dimers from the subject. The result of this depletion is that CFH activity increases, thereby reducing the complement activation, which in turn effectively treats the disease.

The agents of the invention may be used for the treatment, prevention or amelioration of a wide range of diseases that are characterised by excessive complement activation. For example, the agent may be used to treat, prevent or ameliorate meningitis, renal disease, including C3 glomerulopathy, autoimmune disease or inflammation including conditions, such as rheumatoid arthritis, asthma, lupus nephritis, ischemia-reperfusion injury, atypical hemolytic uremic syndrome, thrombotic thrombocytopenic purpura, paroxysmal nocturnal hemoglobinuria, Membranoproliferative glomerulonephritis, hemolytic uremic syndrome, Hypocomplementemic glomerulonephritis, dense deposit disease, macular degeneration (e.g. age-related macular degeneration, AMD), spontaneous foetal loss, Pauci-immune vasculitis, epidermolysis bullosa, recurrent foetal loss, multiple sclerosis, traumatic brain injury, Degos' disease, myasthenia gravis, cold agglutinin disease, dermatomyositis, Graves' disease, Hashimoto's thyroiditis, type I diabetes, psoriasis, pemphigus, autoimmune hemolytic anaemia, idiopathic thrombocytopenic purpura, Goodpasture syndrome, antiphospholipid syndrome, Infective endocarditis, and injury resulting from myocardial infarction, cardiopulmonary bypass and hemodialysis. (See, e.g., Holers et al. (2008) Immunological Reviews 223:300-316). Treatment of AMD or C3 glomerulopathy is particularly preferred.

The complement factor H-related (CFHR) proteins will be well-known to the skilled person, and the DNA and protein sequences of each of these proteins are available on freely accessible databases.

For example, the coding DNA (cDNA) sequence of CFHR1 is 1271 nucleotides long (Accession Number [ensemble.org]: ENSG0000024404), and is provided herein as SEQ ID NO:1, as follows:

[SEQ TD NO: 1]
ATGCTCATAACTGTTAATGAAAGCAGATTCAAAGCAACACCACCACCACTGAAGTATTTTTAGTTATATAAGATT
GGAACTACCAAGCATGTGGCTCCTGGTCAGTGTAATTCTAATCTCACGGATATCCTCTGTTGGGGGAGAAGCAAC
ATTTTGTGATTTTCCAAAAATAAACCATGGAATTCTATATGATGAAGAAAAATATAAGCCATTTTCCCAGGTTCC
TACAGGGGAAGTTTTCTATTACTCCTGTGAATATAATTTTGTGTCTCCTTCAAAATCATTTTGGACTCGCATAAC
ATGCACAGAAGAAGGATGGTCACCAACACCAAAGTGTCTCAGACTGTGTTTCTTTCCTTTTGTGGAAAATGGTCA
TTCTGAATCTTCAGGACAAACACATCTGGAAGGTGATACTGTGCAAATTATTTGCAACACAGGATACAGACTTCA
AAACAATGAGAACAACATTTCATGTGTAGAACGGGGCTGGTCCACCCCTCCCAAATGCAGGTCCACTGACACTTC
CTGTGTGAATCCGCCCACAGTACAAAATGCTCATATACTGTCGAGACAGATGAGTAAATATCCATCTGGTGAGAG
AGTACGTTATGAATGTAGGAGCCCTTATGAAATGTTTGGGGATGAAGAAGTGATGTGTTTAAATGGAAACTGGAC
AGAACCACCTCAATGCAAAGATTCTACGGGAAAATGTGGGCCCCCTCCACCTATTGACAATGGGGACATTACTTC
ATTCCCGTTGTCAGTATATGCTCCAGCTTCATCAGTTGAGTACCAATGCCAGAACTTGTATCAACTTGAGGGTAA
CAAGCGAATAACATGTAGAAATGGACAATGGTCAGAACCACCAAAATGCTTACATCCGTGTGTAATATCCCGAGA
AATTATGGAAAATTATAACATAGCATTAAGGTGGACAGCCAAACAGAAGCTTTATTTGAGAACAGGTGAATCAGC
TGAATTTGTGTGTAAACGGGGATATCGTCTTTCATCACGTTCTCACACATTGCGAACAACATGTTGGGATGGGAA
ACTGGAGTATCCAACTTGTGCAAAAAGATAGAATCAATCATAAAATGCACACCTTTATTCAGAACTTTAGTATTA
AATCAGTTCTTAATTTCATTTTTAAGTATTGTTTTACTCCTTTTTATTCATACGTAAAATTTTGGATTAATTTGT
GAAAATGTAATTATAAGCTGAGACCGGTGGCTCTCTTCTTAAAAGCACCATATTAAAACTTGGAAAACTAA

The protein sequence of CFHR1 is 330 amino acids long (Accession Number [www.ncbi.nlm.nih.gov/], CCDS1386.1), and is provided herein as SEQ ID NO:2, as follows:

[SEQ ID NO:2]
MWLLVSVILISRISSVGGEATFCDFPKINHGILYDEEKYKPFSQVPTGEV
FYYSCEYNFVSPSKSFWTRITCTEEGWSPTPKCLRLCFFPFVENGHSESS
GQTHLEGDTVQIICNTGYRLQNNENNISCVERGWSIPPKCRSTOTSCVNP
PTVQNAHILSRQMSKYPSGERVRYECRSPYEMFGDEEVMCLNGNWTEPPQ
CKDSTGKCGPPPPIDNGDITSFPLSVYAPASSVEYQCQNLYQLEGNKRIT
CRNGQWSEPPKCLHPCVISREIMENYNIALRWTAKQKLYLRTGESAEFVC
KRGYRLSSRSHTLRTTCWDGKLEYPICAKR

The cDNA sequence of CFHR2 is 1062 nucleotides long (Accession Number [ensemble.org]: ENSG00000080910) and is provided herein as SEQ ID NO:3, as follows:

[SEQ ID NO: 3]
CAGTTAGTACACTGAAATTCAAAGTCATGCTCATAACTGTTAATGAAAGC
AGATTCAAAGCAACACCACCACCACTGAAGTATTTTTAGTTATATAAGAT
TGGAACTACCAAGCATGTGGCTCCTGGTCAGTGTAATTCTAATCTCACGG
ATATCCTCTGTTGGGGGAGAAGCAATGTTCTGTGATTTTCCAAAAATAAA
CCATGGAATTCTATATGATGAAGAAAAATATAAGCCATTTTCCCAAGTTC
CTACAGGGGAAGTTTTCTATTACTCCTGTGAATATAATTTTGTGTCTCCT
TCAAAATCCTTTTGGACTCGCATAACGTGCGCAGAAGAAGGATGGTCACC
AACACCAAAGTGTCTCAGACTGTGTTTCTTTCCTTTTGTGGAAAATGGTC
ATTCTGAATCTTCAGGACAAACACATCTGGAAGGTGATACTGTACAAATT
ATTTGCAACACAGGATACAGACTTCAAAACAATGAGAACAACATTTCATG
TGTAGAACGGGGCTGGTCCACTCCTCCCAAATGCAGGTCCACTATTTCTG
CAGAAAAATGTGGGCCCCCTCCACCTATTGACAATGGAGACATTACTTCA
TTCCTGTTGTCAGTATATGCTCCAGGTTCATCAGTTGAGTACCAGTGCCA
GAACTTGTATCAACTTGAGGGTAACAATCAAATAACATGTAGAAACGGAC
AATGGTCAGAACCACCAAAATGCTTAGATCCATGTGTAATATCACAAGAA
ATTATGGAAAAATATAACATAAAATTAAAGTGGACAAACCAACAAAAGCT
TTATTCAAGAACAGGTGACATAGTTGAATTTGTTTGTAAATCTGGATATC
ATCCAACAAAATCTCATTCATTTCGAGCAATGTGTCAGAATGGGAAACTG
GTATATCCCAGTTGTGAAGAAAAATAGAATCAATGGCATTACTATTAGTA
AAATGCACACCTTTTTCTGAATTTACTATTATATTTGTTTTCAATTTCAT
TTTTCAAGTACTGTTTTACTCATTTTTATTCATAAATAAAGTTTTGTGTT
GATTTGTGAAAA

The protein sequence of CFHR2 is 270 amino acids long (Accession Number [www.ncbi.nlm.nih.gov/], CCDS30959.1), and is provided herein as SEQ ID NO:4, as follows:

[SEQ ID NO: 4]
MWLLVSVILISRISSVGGEAMFCDFPKINHGILYDEEKYKPFSQVPTGEV
FYYSCEYNFVSPSKSFWTRITCAEEGWSPTPKCLRLCFFPFVENGHSESS
GQTHLEGDTVQIICNTGYRLQNNENNISCVERGWSTPPKCRSTISAEKCG
PPPPIDNGDITSFLLSVYAPGSSVEYQCQNLYQLEGNNQITCRNGQWSEP
PKCLDPCVISQEIMEKYNIKLKWTNQQKLYSRTGDIVEFVCKSGYHPTKS
HSFRAMCQNGKLVYPSCEEK

The cDNA sequence of CFHR3 is 1645 nucleotides long (Gene Accession Number [ensemble.org]: ENSG00000116785), and is provided herein as SEQ ID NO:5, as follows:

[SEQ ID NO: 5]
GAACCACACTTGGTAACTAATAATGAAAGATTTCAAACCCCAAACAGTGC
AACTGAAACTTTTGTATTAGCATACTACTGAGAATATCTAACATCTTGTT
ACTAATCAATCTCATTCTGACCTTGTGGGTTTCCTGTGCTAATGGACAAG
TGAAACCTTGTGATTTTCCAGACATTAAACATGGAGGTCTATTTCATGAG
AATATGCGTAGACCATACTTTCCAGTAGCTGTAGGAAAATATTACTCCTA
TTACTGTGATGAACATTTTGAGACTCCTTCAGGAAGTTACTGGGATTACA
TTCATTGCACACAAAATGGGTGGTCACCAGCACTACCATGTCTCAGAAAA
TGTTATTTTCCTTATTTGGAAAATGGATATAATCAAAATTATGGAAGAAA
GTTTGTACAGGGTAACTCTACAGAAGTTGCCTGCCATCCTGGCTACGGTC
TTCCAAAAGCGCAGACCACAGTTACATGTACGGAGAAAGGCTGGTCTCCT
ACTCCCAGATGCATCCGTGTCAGAACATGCTCAAAATCAGATATAGAAAT
TGAAAATGGATTCATTTCCGAATCTTCCTCTATTTATATTTTAAATAAAG
AAATACAATATAAATGTAAACCAGGATATGCAACAGCAGATGGAAATTCT
TCAGGATCAATTACATGTTTGCAAAATGGATGGTCACCACAACCAATTTC
CATTAATTCTTCAGAAAACTCTGGCCCTCCTCCACCTATTAGCAATCGTG
ATACCACCTCCTTTCTACTAAAAGTGTATGTGCCACAGTCAAGAGTCGAG
TACCAATGCCAGCCCTACTATGAACTTCAGGGTTCTAATTATGTAACATG
TAGTAATGGAGAGTGGTCGGAACCACCAAGATGCATACATCCATGTATAA
TAACTGAAGAAAACATCAATAAAAATAACATAAAGTTAAAAGGAAGAAGT
GACAGAAAATATTATCCAAAAACAGGGGATACCATTGAATTTATGTGTAA
ATTGGGATATAATGCAAATACATCAATTCTATCATTTCAAGCAGTGTGTC
GGGAAGGGATAGTGGAATACCCCAGATGCGAATAAGGCAGCATTGTTACC
CTAAATGTATGTCCAACTTCCACTTTTCCACTTCTCACTCTTATGGTCTC
AAACCTTCCAAAGATAGCTTCTGATATTCTTGTAATTTCTACTTTATTTC
AAAGAAAATTAATATAATAGTTTCAATTTGCAACTTAATATATTCTCAAA
AATATATTAAAACAAACTAAATTATTGCTTATCCTTGTACTAAAATAATA
AAAACTACTCTTATATTGGACTTCTTATCAATCAATTAGTAAGTATAGAG
ACAGACAGCTGAATGGCTTTCTGCATATTGTATAGTATACCTAGACATAG
AAACAAAATGACTTTAGATTTTATTTGGGGAAGTAATAATACCATAAAAT
TAGATATTAAAATTGTAAGTGAAGATAAACACACTATAGTATTCCCTTAT
TGTACCCATGGTCCTCTAGATGCACTTAACCAAATAGGGTCATTTTTATT
AAAAGTACTGTTTCCTGGCAAACACTGACATTACATCATTATCATGATTT
AAAGGAAATAGTACTAGAGAAGGTGAATTATTATCATTTTCCTGT

The protein sequence of CFHR3 is 330 amino acids long (Accession Number [www.ncbi.nlm.nih.gov/], CCDS30958.1), and is provided herein as SEQ ID NO:6, as follows:

[SEQ ID NO: 6]
MLLLINVILILWVSCANGQVKPCDFPDIKHGGLEHENMRRPYFPVAVGKY
YSYYCDEHFETPSGSYWDYIHCTQNGWSPAVPCLRKCYFPYLENGYNQNY
GRKFVQGNSTEVACHPGYGLPKAQTTVTCTEKGWSPTPRCIRVRTCSKSD
IEIENGFISESSSIYILNKEIQYKCKPGYATADGNSSGSITCLQNGWSAQ
PICINSSEKCGPPPPISNGDTTSFLLKVYVPQSRVEYQCQPYYELQGSNY
VTCSNGEWSEPPRCIHPCIITEENMNKNNIKLKGRSDRKYYAKTGDTIEF
MCKLGYNANTSILSFQAVCREGIVEYPRCE

The cDNA sequence of CFHR4 is 1292 nucleotides long (Gene Accession Number [ensemble.org]: ENSG00000134365), and is provided herein as SEQ ID NO:7, as follows:

[SEQ ID NO: 7]
TGAAAGATTTCAAACCCCAAACAGTGCAACTGAAACTTTTGCATTACTAT
ACTACTGAGAATATCTAACATGTTGTTACTAATCAATGTCATTCTGACCT
TGTGGCTTTCCTGTGCTAATCGACAAGCAATGAAACCTTGTGAGTTTCCA
GAAATTCAACATGGACATCTATATTATGAGAATACGCGTAGACCATACTT
TCCAGTAGCTACAGGACAATCTTACTCCTATTACTGTGACCAAAATTTTG
TGACTCCTTCAGGAAGTTACTGGGATTACATTCACTGCACACAAGATGGG
TGGTTGCCAACAGTCCCATGCCTCAGAACATGCTCAAAATCAGATATAGA
AATTGAAAATGGATTCATTTCTGAATCTTCCTCTATTTATATTTTAAATA
AAGAAATACAATATAAATGTAAACCAGGATATGCAACAGCAGATGGAAAT
TCTTCAGGTTCAATTACATGTTTGCAAAATGGATGGTCAGCACAACCAAT
TTGCATTAAATTTTGTGATATGCCTGTTTTTGAGAATTCCAGAGCCAAGA
GTAATGGCATGCGGTTTAAGCTCCATGACACATTGGACTACGAATGCTAC
GATGGATATGAAATCAGTTATGGAAACACCACAGGTTCCATAGTGTGTGG
TGAAGATGGGTGGTCCCATTTCCCAACATGTTATAATTCTTCAGAAAAGT
GTGGGCCTCCTCCACCTATTAGCAATGGTGATACCACCTCCTTTCTACTA
AAAGTGTATGTGCGACAGTCAAGAGTCGAGTACGAATGCCAGTCCTACTA
TGAACTTCAGGGTTCTAATTATGTAACATGTAGTAATGGAGAGTGGTCGG
AACCACCAAGATGCATACATCCATGTATAATAACTGAAGAAAACATGAAT
AAAAATAACATACAGTTAAAAGGAAAAAGTGACATAAAATATTATGCAAA
AACAGGGGATACCATTGAATTTATGTGTAAATTGGGATATAATGCGAATA
CATCAGTTCTATCATTTCAAGCAGTGTGTAGGGAAGGCATAGTGGAATAC
CCCAGATGCGAATAAGGCAGCATTGTTACCCTAAATGTATGTCCAACTTC
CACTTCTCACTCTTATGGTCTCAAAGCTTGCAAAGATAGCTTCTGATATT
GTTGTAATTTCTACTTTATTTCAAAGAAAATTAATATAATAGTTTCAATT
TGCAACTTAATATGTTCTCAAAAATATGTTAAAACAAACTAAATTATTGC
TTATGCTTGTACTAAAATAATAAAAACTACCCTTATATTGGA

CFHR4 exists as two isoforms termed CFHR4A and CFHR4B. The protein sequence of CFHR4A (577 amino acids) [www.ncbi.nlm.nih.gov/], CCDS55671.1, is provided herein as SEQ ID NO:8, as follows:

[SEQ ID NO: 8]
MLLLINVILTLWVSCANGQVKPCDFPEIQHGGLYYKSLRRLYFPAAAGQS
YSYYCDQNFVTPSGSYWDYIHCTQDGWSPTVPCLRTCSKSDVEIENGFIS
ESSSIYILNEETQYNCKPGYATAEGNSSGSITCLQNGWSTQPICIKFCDM
PVFENSRAKSNGMWFKLHDTLDYECYDGYESSYGNTTDSIVCGEDGWSHL
PTCYNSSENCGPPPPISNGDTTSFPQKVYLPWSRVEYQCQSYYELQGSKY
VTCSNGDWSEPPRCISMKPCEFPEIQHGHLYYENTRRPYFPVATGQSYSY
YCDQNFVTPSGSYWDYIHCTQDGWLPTVPCLRTCSKSDIEIENGFISESS
SIYILNKEIQYKCKPGYATADGNSSGSITCLQNGWSAQPICIKECDMPVF
ENSRAKSNGMRFKLHDTLDYECYDGYEISYGNTTGSIVCGEDGWSHFPTC
YNSSEKCGPPPPISNGDTTSFLLKVYVPQSRVEYQCQSYYELQGSNYVTC
SNGEWSEPPRCIHPCIITEENMNKNNIQLKGKSDIKYYAKTGDTIEFMCK
LGYNANTSVLSFQAVCREGIVEYPRCE

The protein sequence of CFHR4B (331 amino acids) [www.ncbi.nlm.nih.gov/], CCDS4145.1, is provided herein as SEQ ID NO:9, as follows:

[SEQ ID NO: 9]
MLLLINVILTLWVSCANGQEVKPCDFPEIQHGGLYYKSLRRLYFPAAAGQ
SYSYYCDQNFVTPSGSYWDYIHCTQDGWSPTVPCLRTCSKSDIEIENGFI
SESSSIYILNKEIQYKCKPGYATADGNSSGSITCLQNGWSAQPICIKFCD
MPVFENSRAKSNGMRFKLHDTLDYECYDGYEISYGNTTGSIVCGEDGWSH
FPTCYNSSEKCGPPPPISNGDTTSFLLKVYVPQSRVEYQCQSYYELQGSN
YVTCSNGEWSEPPRCIHPCIITEENMNKNNIQLKGKSDIKYYAKTGDTIE
FMCKLGYNANTSVLSFQAVCREGIVEYPRCE

The cDNA sequence of CFHR5 is 2810 nucleotides long (Gene Accession Number [ensemble.org]: ENSG00000134389), and is provided herein as SEQ ID NO:10, as follows:

[SEQ ID NO: 10]
AGTACATTGAAATTCAAAGTCATGCTTGTAACTGTTAATGAAAGCAGATT
TAAAGCAACACCACCATCACTGGAGTATTTTTAGTTATATACGATTGAGA
CTACCAAGCATGTTGCTCTTATTCAGTGTAATCCTAATCTCATGGGTATC
CACTGTTGGGGGAGAAGGAACACTTTGTGATTTTCCAAAAATACACCATG
GATTTCTGTATGATGAAGAAGATTATAACCCTTTTTCCCAAGTTCCTACA
GGGGAAGTTTTCTATTACTCCTGTGAATATAATTTTGTGTCTCCTTCAAA
ATCCTTTTGGACTCGCATAACATGCACAGAAGAAGGATGGTCACCAACAC
CGAAGTGTCTCAGAATGTGTTCCTTTCCTTTTGTGAAAAATGGTCATTCT
GAATCTTCAGGACTAATACATCTGGAAGGTGATACTGTACAAATTATTTG
CAACACAGGATACAGCCTTCAAAACAATGAGAAAAACATTTCGTGTGTAG
AACGGGGCTGGTCCACTCCTCCCATATGCAGCTTCACTAAAGGAGAATCT
CATGTTCCAATTTTAGAACCCAATGTAGATGCTCAGCCAAAAAAAGAAAG
CTACAAAGTTGGAGACGTGTTGAAATTCTCCTGCAGAAAAAATCTTATAA
GAGTTGGATCAGACTCAGTTCAATGTTACCAATTTGGGTGGTCACCTAAC
TTTCCAACATGCAAAGGACAAGTACGATCATGTGGTCCACCTCCTCAACT
CTCCAATGGTGAAGTTAAGGAGATAAGAAAAGAGGAATATGGACACAATG
AAGTAGTGGAATATGATTGCAATCCTAATTTTATAATAAACGGGCCTAAG
AAAATACAATGTGTGGATGGAGAATGGACAACTTTACCCACTTGTGTTGA
ACAAGTGAAAACATGTGGATACATACCTGAACTCGAGTACGGTTATGTTC
AGCCGTCTGTCCCTCCCTATCAACATGGAGTTTCAGTCGAGGTGAATTGC
AGAAATGAATATGCAATGATTGGAAATAACATGATTACCTGTATTAATGG
AATATGGACAGAGCTTCCTATGTGTGTTGCAACACACCAACTTAAGAGGT
GCAAAATAGCAGGAGTTAATATAAAAACATTACTCAAGCTATCTGGGAAA
GAATTTAATCATAATTCTAGAATACGTTACAGATGTTCAGACATCTTCAG
ATACAGGCACTCAGTCTGTATAAACGGGAAATGGAATCCTGAAGTAGACT
GCACAGAAAAAAGGGAACAATTCTGCCCACCGCCACCTCAGATACCTAAT
GCTCAGAATATGACAACCACAGTGAATTATCAGGATGGAGAAAAAGTAGC
TGTTCTCTGTAAAGAAAACTATCTACTTCCAGAAGCAAAAGAAATTGTAT
GTAAAGATGGACGATGGCAATCATTACCACGCTGTGTTGAGTCTACTGCA
TATTGTGGGCCCCCTCCATCTATTAACAATGGAGATACCACCTCATTCCC
ATTATCAGTATATCCTCCAGGGTCAACAGTGACGTACCGTTGCCAGTCCT
TCTATAAACTCCAGGGCTCTGTAACTGTAACATGCAGAAATAAACAGTGG
TCAGAACCACCAAGATGCCTAGATCCATGTGTGGTATCTGAAGAAAACAT
GAACAAAAATAACATACAGTTAAAATGGAGAAACGATGGAAAACTCTATG
CAAAAACAGGGGATGCTGTTGAATTCCAGTGTAAATTCCCACATAAAGCG
ATGATATCATCACCACCATTTCGAGCAATCTGTCAGGAAGGGAAATTTGA
ATATCCTATATGTGAATGAAGCAAGCATAATTTTCCTGAATATATTCTTC
AAACATCCATCTATGCTAAAAGTAGCCATTATGTAGCCAATTCTGTAGTT
ACTTCTTTTATTCTTTCAGGTGTTGTTTAACTCAGTTTTATTTAGAACTC
TGGATTTTTAGAGCTTTAGAAATTTGTAAGCTGAGAGAACAATGTTTCAC
TTAATAGGAGGGTGTCTTAGTCCATATTACATTGTTATAACAGAGTATCA
CAGACTGGATAACTTCTAACCAATAGTTTATTTGTTTCATAAATCTAAAA
GCTGAGAAGTCCAAGATGGTGGGGCTGCCTCTGGTGAGGGTCTTCTCGAA
GCATCATAATATGCTGGAAGGCATCACAACATGGTGGAAGGGATCACGTG
GCAAAAGAGCATGTACATGGGAGTGAGAGAAAAAGAGAGAGAGAGACAGA
GTGGCGGGGGCGGGGAGGAGCGCAAACTCATCCTTTATAAAGACACCACT
CCTGAGATAACAATCCAATCCCATGATAATGACATTAATCCATTCAAGAA
GATAGAGCTCTCGTGACTTAATCACCTTCTAAAGATCTCACCTGACAACA
CTGTTGCATTGGCAGTTAAGTTTCCACGTAAACTTTCGGGGACACATTCA
AACCACAGGAGAAACTCAAATTGTTCCTGGGCAAATCACAACATGGGGAA
TTTTATTCATAAATGTCCACAGAAACAGTAAATGTTCTCGCTTCAGTACT
TAATTCATCTAATCCCTCCTGTTTGTCTCAAATTATAGGATAACTTTGAA
ACTTTCTGAATTAACGTTATTTAAAAGGAAATGTAGATGTTATTTTAGTC
TCTATCTTCATGTTATTATCACTTAAAAACCTGCGAAAGCTGTCAACTTT
TGTGGTTGTAGCAAGTATTAATAAATATTTATAAATCCTCTAATGTAAGT
CTAGCTACCTATCCAATACTAAATACCCCTTAAAGTATTAAATGCACTAT
CTGCTGTAAA

The protein sequence of CFHR5 is 569 amino acids long (Accession Number [www.ncbi.nlm.nih.gov/], CCDS1387.1), and is provided herein as SEQ ID NO:11, as follows:

[SEQ ID NO: 11]
MLLLFSVILISWVSTVGGEGTLCDFPKIHHGFLYDEEDYNPFSQVPTGEV
FYYSCEYNFVSPSKSFWTRITCTEEGWSPIPKCLRMCSFPFVKNGHSESS
GLIHLEGDTVQIICNTGYSLQNNEKNISCVERGWSTPPICSFTKGECHVP
ILEANVDAQPKKESYKVGDVLKFSCRKNLIRVGSDSVQCYQFGWSPNFPT
CKGQVRSCGPPPQLSNGEVKEIRKEEYGHNEVVEYDCNPNFIINGPKKIQ
CVDGEWTTLPTCVEQVKTCGYIPELEYGYVQPSVPPYQHGVSVEVNCRNE
YAMIGNNMITCINGIWTELPMCVATHQLKRCKIAGVNIKTLLKLSGKEFN
HNSRIRYRCSDIFRYRHSVCINGKWNPEVDCTEKREQFCPPPPQIPNAQN
MTTTVNYQDGEKVAVLCKENYLLPEAKEIVCKDGRWQSLPRCVESTAYCG
PPPSINNGDTTSFPLSVYPPGSTVTYRCQSFYKLQGSVTVTCRNKQWSEP
PRCLDPCVVSEENMNKNNIQLKWRNDGKLYAKTGDAVEFQCKFPHKAMIS
SPPFRAICQEGKFEYPICE

Therefore, reference herein to each of CFHR1-5 is preferably to the various Accession Numbers disclosed herein, and to functional variants and fragments thereof. Accordingly, agents of the invention may reduce the concentration or activity of, or reduce or inhibit dimerisation or higher order assembly of, at least one CFHR protein comprising an amino acid sequence substantially as set out in SEQ ID NO:2, 4, 6, 8, 9 or 11, or a functional variant or fragment thereof. The CFHR protein may be encoded by a nucleic acid sequence substantially as set out in SEQ ID No: 1, 3, 5, 7 or 10, or a functional variant or fragment thereof.

Preferably, the agent binds to domain 1 and 2 (i.e. the first 120 amino acids of each protein) of any of SEQ ID NO:2, 4, 6, 8, 9 or 11, or a fragment of variant thereof, and thereby reduces the concentration or activity of, or reduces or inhibits dimerisation or higher order assembly of, the at least one CFHR protein. Domains 1 and 2 are believed to be exposed in vivo and so would act as a useful binding partner for the agent. However, it is preferred that the agent is capable of binding specifically against the dimerisation motif described herein, which is shown in FIG. 1c.

The inventors have produced a sequence alignment between CFHR1, CFHR2 and CFHR5 in the dimerisation domains, which is shown below:—

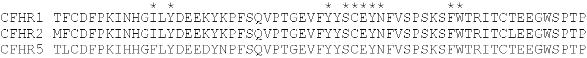

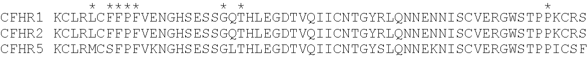

Residues which differ between the proteins are highlighted (red—non-conservative change, green—conservative change). Residues involved in dimer formation are indicated above the sequence alignment by •. In the above sequence alignment, the amino acid sequence for CFHR1 is referred to herein as SEQ ID No. 22, the amino acid sequence for CFHR2 is referred to herein as SEQ ID No. 23, and the amino acid sequence for CFHR5 is referred to herein as SEQ ID No. 24. Using this alignment, the inventors have created a consensus sequence, as shown in SEQ ID No.12, as follows.

[SEQ ID NO: 12]
PFSQVPTGEVFYYSCEYNFVSPSKSFWTRITC

Preferably, therefore, the agent may bind to a region within the sequence alignment represented above, most preferably SEQ ID No.12, or a fragment or variant thereof, and thereby reduces the concentration or activity of, or reduces or inhibits dimerisation or higher order assembly of, the at least one CFHR protein. Preferably, the at least one CFHR protein is CFHR1, 2 or 5. The inventors determined the crystal structure of the first two SCR domains of CFHR1 (CFHR1,2), which revealed that these domains assemble as a tight head-to-tail dimer with residues Tyr34, Ser36 and Tyr39 identified in SEQ ID No:22, 23 or 24, and SEQ ID NO:12, playing key roles in stabilising the assembly (See FIGS. 1b-d, Table 1). Hence, the inventors have established that the Tyr34, Ser36 and Tyr39 residues located within SEQ ID No:22, 23 or 24, and SEQ ID NO.12 are important for stabilising the CFHR dimers, and just this important section of the dimerisation motif is provided herein as SEQ ID NO:13, as follows:

[SEQ ID NO: 13]
YYSCEYN

Hence, it is preferred that the agent binds to SEQ ID No.13 (and especially Tyr34, Ser36 and Tyr39 residues thereof), or a fragment or variant thereof, and thereby reduces the concentration or activity of, or reduces or inhibits dimerisation or higher order assembly of, the at least one CFHR protein. Preferably, the at least one CFHR protein is CFHR1, 2 or 5. The inventors believe however that, in some cases, and under certain conditions, SEQ ID No.13 may not always be exposed in vivo, and so in some embodiments of the invention, the agent may bind to a region within the sequence alignment represented above, most preferably SEQ ID NO.12, or a fragment or variant thereof, other than that which is represented by SEQ ID No.13, and thereby reduces the concentration or activity of, or reduces or inhibits dimerisation or higher order assembly of, the at least one CFHR protein. Hence, the agent targets the dimerisation motif in order to clear (i.e. reduce the concentration) the CFHR dimers from the patient, but may not actually prevent dimerisation per se.

The inventors have also produced a sequence alignment between CFHR3 (short consensus repeat domain number three) and CFHR4 (short consensus repeat domain number two), which is shown below:—

CFHR3-
RTCSKSDIEIENGFISESSSIYILNKEIQYKCKPGYATADGNSSGSITCL
QNGWSAQPICIN
CFHR4-
RTCSKSDIEIENGFISESSSIYILNKEIQYKCKPGYATADGNSSGSITCL
QNGWSAQPICIK

In the above sequence alignment, the amino acid sequence for CFHR3 is referred to herein as SEQ ID No. 25, and the amino acid sequence for CFHR4 is referred to herein as SEQ ID No. 26. Using this alignment, they have created a consensus sequence, as shown in SEQ ID No.27, as follows.

[SEQ ID NO: 27]
RTCSKSDIEIENGFISESSSIYILNKEIQYKCKPGYATADGNSSGSITCL
QNGWSAQPICI

Accordingly, it is preferred that the agent may bind to a region within SEQ ID No.27, or a fragment or variant thereof, and thereby reduces the concentration or activity of, or reduces or inhibits dimerisation or higher order assembly of, the at least one CFHR protein. Preferably, the at least one CFHR protein is CFHR3 or CFHR4.

The agent may reduce the concentration or activity of a dimer or higher order assembly of the CFHR. For example, in one embodiment, the dimer may be a homodimer selected from a group consisting of: CFHR1-CFHR1, CFHR2-CFHR2, CFHR3-CFHR3, CFHR4-CFHR4 and CFHR5-CFHR5. Preferred homodimers which are targeted by the agent may include CFHR1-CFHR1, CFHR2-CFHR2 or CFHR5-CFHR5.

In another embodiment, however, the dimer may be a heterodimer selected from a group consisting of: CFHR1-CFHR2, CFHR1-CFHR3, CFHR1-CFHR4, CFHR1-CFHR5, CFHR2-CFHR3, CFHR2-CFHR4, CFHR2-CFHR5, CFHR3-CFHR4, CFHR3-CFHR5 and CFHR4-CFHR5. Preferred heterodimers may include CFHR1-CFHR2, CFHR1-CFHR5 and CFHR2-CFHR5.

In yet another preferred embodiment, however, the dimer may be a heterodimer selected from a group consisting of: CFHR1-CFHR2, CFHR1-CFHR5, CFHR2-CFHR5 and CFHR3-CFHR4. Preferred heterodimers may include CFHR1-CFHR2, CFHR1-CFHR5 and CFHR2-CFHR5.

The agent may reduce the concentration or activity of, or reduce or inhibit dimerisation or higher order assembly of, at least two, three, four or five CFHR proteins selected from a group consisting of: CFHR1, CFHR2, CFHR3, CFHR4 and CFHR5, or homo- or heterodimers thereof. Thus, the agent can reduce the concentration or activity of, or reduce or inhibit dimerisation or higher order assembly of CFHR1 homo- and heterodimers, CFHR2 homo- and heterodimers, CFHR3 homo- and heterodimers, CFHR4 homo- and heterodimers and/or CFHR5 homo- or heterodimers. The agent can reduce the concentration or activity of, or reduce or inhibit dimerisation or higher order assembly of trimers and tetramers.

The inventors have found to their surprise that reducing the concentration of CFHR's or homo- or heterodimers thereof, or reducing or inhibiting activity of the CFHR's or their dimers, results in a decrease of complement activation, which is required for the effective treatment of certain diseases. This came as a surprise, because it is the opposite of what is taught in the prior art (Heinen S, et al., “Factor H-related protein 1 (CFHR-1) inhibits complement C5 convertase activity and terminal complex formation”. Blood. 2009 Sep. 17; 114(12):2439-47. doi: 10.1182/blood-2009-02-205641. Epub 2009 Jun. 15. PubMed PMID: 19528535; McRae J L, et al., “Human factor H-related protein 5 has cofactor activity, inhibits C3 convertase activity, binds heparin and C-reactive protein, and associates with lipoprotein”. J. Immunol. 2005 May 15; 174(10):6250-6. PubMed. PMID: 15879123).

Reduction of protein concentration can be referred to as protein depletion, and reduction of protein activity can be referred to as protein neutralisation or inhibition.

Based on the detailed structure of the CFHR, CFH and C3 fragments (including C3b, iC3b, C3d and C3dg) complexes shown in the Figures and described in the Examples, the skilled person would readily appreciate how a suitable agent could be prepared, which would be capable of locating itself in such a way that CFHR binding with C3 fragments is inhibited, such that complement activation is reduced. In one embodiment, the agent (which could be referred to as an inhibitor), which is capable of reducing the concentration or activity of a CFHR protein, may achieve its effect by a number of means. For instance, the agent may:—
(a) reduce binding between a CFHR and a C3 fragment;
(b) increase binding between CFH and a C3 fragment;
(c) bind to a CFHR to reduce its biological activity; or
(d) decrease expression of a CFHR.

“CFHR” as used herein may refer to one or more of CFHR1-5, and “a C3 fragment” may include C3b, iC3b, C3d and/or C3dg.

In another embodiment, the agent may be capable of reducing or inhibiting dimerisation or higher order assembly of a CFHR protein.

A number of different agents may be used according to the invention. For example, the agent may comprise a competitive polypeptide or a peptide-like molecule, or a derivative or analogue thereof; an antibody or antigen-binding fragment or derivative thereof; an aptamer (nucleic acid or peptide); a peptide-binding partner; or a small molecule that binds specifically to the CFHR protein to prevent it binding to a C3 fragment. The agent may comprise a small molecule having a molecule weight of less than 1000 Da.

The term “derivative or analogue thereof” can mean a polypeptide within which amino acids residues are replaced by residues (whether natural amino acids, non-natural amino acids or amino acid mimics) with similar side chains or peptide backbone properties. Additionally, either one or both terminals of such peptides may be protected by N- and C-terminal protecting groups, for example groups with similar properties to acetyl or amide groups. It will be appreciated that the amino acid sequence may be varied, truncated or modified once the final polypeptide is formed or during the development of the peptide.

According to another embodiment of the invention, short peptides may be used to inhibit interaction or binding between CFHR and a C3 fragment, to prevent the complex forming. These peptides may be isolated from libraries of peptides by identifying which members of the library are able to bind to the peptide of SEQ ID NO:2, 4, 6, 8, 9, or 11, or a fragment of variant thereof. Suitable libraries may be generated using phage display techniques (e.g. as disclosed in Smith & Petrenko (1997) Chem Rev 97 p 391-410).

In a preferred embodiment, however, the agent may comprise an antibody, or antigenic binding fragment thereof. The antibody may be a neutralising antibody, which may be capable of neutralising and/or clearing CFHR proteins, or dimers or higher order assemblies thereof, from the subject. The antibody may be polyclonal or monoclonal. Polyclonal antibodies according to the invention may be produced as polyclonal sera by injecting antigen into animals. Preferred polyclonal antibodies may be raised by inoculating an animal (e.g. a rabbit) with antigen (e.g. a CFHR homo- or heterodimer, or a fragment thereof) using techniques known to the art. Polyclonal antibodies, for use in treating human subjects, may be raised against a number of epitopes described herein.

Conventional hybridoma techniques may be used to raise monoclonal antibodies. The skilled person will know how monoclonal antibodies specific for the dimerisation motif can be generated. For example, using a construct consisting of only the assembled dimerisation motif (e.g. CFHR1-domains 1 & 2, CFHR2-domains 1 & 2, CFHR5-domains 1 & 2, or any combination thereof) to immunise animals provides a generic way to generate antibodies targeting this region of the protein. The antigen used to generate monoclonal antibodies may be the whole CFHR protein or only a fragment thereof.

Hence, antibodies, for use in treating human subjects, may be raised against any of SEQ ID NO:2, 4, 6, 8, 9 or 11, or a fragment of variant thereof, acting as antigen. Preferably, domains 1 and 2 of any of SEQ ID NO:2, 4, 6, 8, 9 or 11, or a fragment of variant thereof, acting as antigen. Domains 1 and 2 are believed to be exposed in vivo and so would act as a useful epitope in antibody engineering. However, it is preferred that the antibody is raised specifically against the dimerisation motif described herein, which is shown in FIG. 1c. The antibody or antigen binding fragment thereof may be raised against regions in the sequence alignments for CFHR 1, 2 and 5 (i.e. preferably SEQ ID No.12 or SEQ ID No.13), and for CFHR 3 and 4 (i.e. preferably SEQ ID No.27), acting as antigen.

A preferred antibody which may be used as an agent of the invention may be known as “2C6”, which is available from Dr Claire Harris, University of Cardiff (Malik T H, et al. (2012) A Hybrid CFHR3-1 Gene Causes Familial C3 Glomerulopathy. J Am Soc Nephrol.).

In a fourth aspect, there is provided an antibody or antigen binding fragment thereof, which binds specifically to SEQ ID No.12, or SEQ ID No. 27, or a fragment or variant thereof.

The antibody or antigen binding fragment thereof may bind specifically to SEQ ID No.13, or a fragment or variant thereof. However, the antibody or antigen binding fragment thereof may bind specifically to a region of SEQ ID No.12, or a fragment or variant thereof, other than that which is represented by SEQ ID No.13.

The antibody or fragment thereof may selectively interact with its epitope with an affinity constant of approximately 10⁻⁵ to 10⁻¹³ M⁻¹, preferably 10⁻⁶ to 10⁻⁹ M⁻¹, even more preferably, 10⁻¹⁰ to 10⁻¹²M⁻¹.

In a fifth aspect, there is provided an antibody or antigen binding fragment according to the fourth aspect, for use in reducing the concentration or activity of, or reducing or inhibiting dimerisation or higher order assembly of, at least one CFHR protein selected from a group consisting of: CFHR1, CFHR2, CFHR3, CFHR4 and CFHR5.

In a sixth aspect, there is provided an antibody or antigen binding fragment according to the fourth aspect, for use in the treatment, prevention or amelioration of a disease characterised by excessive complement activation.

Identification of the dimerisation motif shown as SEQ ID No.12, and especially the central portion thereof shown as SEQ ID No.13, and the motif shown as SEQ ID No.27, is an important aspect of the invention.

Thus, in a seventh aspect, there is provided use of SEQ ID NO:12 or SEQ ID No:13 or SEQ ID No: 27 or a functional variant or fragment thereof, as an epitope for generating an antibody, or a functional fragment thereof.

Preferably, SEQ ID NO:13 is used as an epitope for producing the antibody.

It is preferred that the antibody is a γ-immunoglobulin (IgG). It will be appreciated that the variable region of an antibody defines the specificity of the antibody and as such this region should be conserved in functional derivatives of the antibody according to the invention. The regions beyond the variable domains (C-domains) are relatively constant in sequence. It will be appreciated that the characterising feature of antibodies according to the invention is the V_{H} and V_{L} domains. It will be further appreciated that the precise nature of the C_{H} and C_{L} domains is not, on the whole, critical to the invention. In fact preferred antibodies according to the invention may have different C_{H} and C_{L} domains.

The inventors have found that antibodies, or functional derivatives thereof, have surprising efficacy for recognising the common dimerisation domain in CFHR proteins, and thereby reduce or prevent their dimerisation or higher order assembly, and thereby reduce complement activation, and so are useful for treating disease.

The antibody may be recombinant and may be chimeric, humanised or fully human. Antibody fragments may include fragments selected from a group consisting of VH (Heavy chain variable region), VL (Light chain variable region), Fd, Fv, Fab, Fab′, scFv, F (ab′)₂ and Fc fragment.

An antibody derivative may have 75% sequence identity, more preferably 90% sequence identity and most preferably has at least 95% sequence identity to a monoclonal antibody or specific antibody in a polyclonal mix. It will be appreciated that most sequence variation may occur in the framework regions (FRs) whereas the sequence of the CDRs of the antibodies, and functional derivatives thereof, is most conserved.

A number of preferred antibodies have both Variable and Constant domains. However it will be appreciated that antibody fragments (e.g. scFV antibodies) are also encompassed by the invention that comprise essentially the Variable region of an antibody without any Constant region. Antibodies generated in one species are known to have several drawbacks when used to treat a different species. For instance, when rodent antibodies are used in humans, they tend to have a short circulating half-life in serum and may be recognised as foreign proteins by the patient being treated. This leads to the development of an unwanted human anti-rodent antibody response. This is particularly troublesome when frequent administrations of the antibody are required as it can enhance the clearance thereof, block its therapeutic effect, and induce hypersensitivity reactions. Accordingly, preferred antibodies (if of non-human source) for use in human therapy are humanised.

Monoclonal antibodies are preferably generated by the well-known hybridoma technique. This usually involves the generation of non-human mAbs. The technique enables rodent monoclonal antibodies to be produced with almost any specificity. Accordingly, preferred embodiments of the invention may use such a technique to develop monoclonal antibodies against CFHR proteins. Although such antibodies are useful, it will be appreciated that such antibodies are not ideal therapeutic agents in humans (as suggested above). Ideally, human monoclonal antibodies would be the preferred choice for therapeutic applications. However, the generation of human mAbs using conventional cell fusion techniques has not always been very successful. The problem of humanisation may be at least partly addressed by engineering antibodies that use V region sequences from non-human (e.g. rodent) mAbs and C region (and ideally FRs from V region) sequences from human antibodies. The resulting ‘engineered’ mAbs are less immunogenic in humans than the rodent mAbs from which they were derived and so are better suited for clinical use.

Humanised antibodies may be chimaeric monoclonal antibodies, in which, using recombinant DNA technology, rodent immunoglobulin constant regions are replaced by the constant regions of human antibodies. The chimaeric H chain and L chain genes may then be cloned into expression vectors containing suitable regulatory elements and induced into mammalian cells in order to produce fully glycosylated antibodies. By choosing an appropriate human H chain C region gene for this process, the biological activity of the antibody may be pre-determined. Such chimaeric antibodies offer advantages over non-human monoclonal antibodies in that their ability to activate effector functions can be tailored for cancer therapy, and the anti-globulin response they induce is reduced.

Such chimaeric molecules are preferred agents and inhibitors for treating diseases characterised by excessive complement activation. RT-PCR may be used to isolate the VH and V_{L} genes from preferred mAbs, cloned and used to construct a chimaeric version of the mAb possessing human domains. Further humanisation of antibodies may involve CDR-grafting or reshaping of antibodies. Such antibodies are produced by transplanting the heavy and light chain CDRs of a rodent mAb (which form the antibody's antigen binding site) into the corresponding framework regions of a human antibody.

In another embodiment, the agent may prevent or reduce expression of CFHR (i.e. feature (d) mentioned above). For example, the agent may be a gene-silencing molecule.

The term “gene-silencing molecule” can mean any molecule that interferes with the expression of any of the CFHR1-5 genes to prevent or reduce their expression. Such molecules include, but are not limited to, RNAi molecules, including siNA, siRNA, miRNA, ribozymes and antisense molecules. The use of such molecules represents an important aspect of the invention.

Therefore, according to a eighth aspect of the present invention, there is provided a complement factor H-related (CFHR) gene-silencing molecule, for use in the treatment, amelioration or prevention of a disease characterised by excessive complement activation.

The gene-silencing molecule may reduce expression of at least one CFHR protein selected from a group consisting of: CFHR1, CFHR2, CFHR3, CFHR4 and CFHR5.

Gene-silencing molecules may be antisense molecules (antisense DNA or antisense RNA) or ribozyme molecules. Ribozymes and antisense molecules may be used to inhibit the transcription of the CFHR1-5 genes. Antisense molecules are oligonucleotides that bind in a sequence-specific manner to nucleic acids, such as DNA or RNA. When bound to mRNA that has a complimentary sequence, antisense RNA prevents translation of the mRNA. Triplex molecules refer to single antisense DNA strands that bind duplex DNA forming a colinear triplex molecule, thereby preventing transcription. Particularly useful antisense nucleotides and triplex molecules are ones that are complimentary to, or bind, the sense strand of DNA (or mRNA) that encodes CFHR1-5.

The expression of ribozymes, which are enzymatic RNA molecules capable of catalysing the specific cleavage of RNA substrates, may also be used to block protein translation. The mechanism of ribozyme action involves sequence specific hybridisation of the ribozyme molecule to complementary target RNA, followed by endonucleolytic cleavage, e.g. hammerhead motif ribozymes.

It is preferred that the gene-silencing molecule is a short interfering nucleic acid (siNA). The siNA molecule may be double-stranded and therefore comprises a sense and an antisense strand. The siNA molecule may comprise an siDNA molecule or an siRNA molecule. However, it is preferred that the siNA molecule comprises an siRNA molecule. Hence, the siNA molecule according to the invention preferably down-regulates gene expression by RNA interference (RNAi).

RNAi is the process of sequence specific post-transcriptional gene-silencing in animals and plants. It uses small interfering RNA molecules (siRNA) that are double-stranded and homologous in sequence to the silenced (target) gene. Hence, sequence specific binding of the siRNA molecule with mRNAs produced by transcription of the target gene allows very specific targeted ‘knockdown’ of gene expression. Preferably, the siNA molecule is substantially identical with at least a region of the coding sequence of the CFHR gene (see above) to enable down-regulation of the gene. One could target any discriminatory exon, using a commercially available siRNA, for example at http://bioinfo.invitrogen.com/genome-database/details/sirna/s37575#assay-details-section.

Preferably, the degree of identity between the sequence of the siNA molecule and the targeted region of the CFHR gene is at least 60% sequence identity, preferably at least 75% sequence identity, preferably at least 85% identity, preferably at least 90% identity, preferably at least 95% identity, preferably at least 97% identity, and most preferably at least 99% or 100% identity. The siNA molecule may comprise between approximately 5 bp and 50 bp, more preferably between 10 bp and 35 bp, even more preferably between 15 bp and 30 bp, and yet still more preferably, between 16 bp and 25 bp. Most preferably, the siNA molecule comprises less than 22 bp.

Aptamers represent another preferred agent for use according to the invention. Aptamers are nucleic acid or peptide molecules that assume a specific, sequence-dependent shape and bind to specific target ligands based on a lock-and-key fit between the aptamer and ligand. Typically, aptamers may comprise either single- or double-stranded DNA molecules (ssDNA or dsDNA) or single-stranded RNA molecules (ssRNA). Peptide aptamers consist of a short variable peptide domain, attached at both ends to a protein scaffold. Aptamers may be used to bind both nucleic acid and non-nucleic acid targets. It is known that the binding of any of the CFHR1-5 homo- or heterodimers to C3b prevents CFH from binding, and thereby de-regulates complement activation. Thus, blocking binding between CFHR1-5 and C3 fragment (e.g. C3b) is preferred. Accordingly, the aptamer may recognise the “half-binding pocket” on either the C3 molecule or CFHR1-5. Accordingly aptamers may be generated.

Suitable aptamers may be selected from random sequence pools, from which specific aptamers may be identified which bind to the selected target molecules (e.g. a peptide of SEQ ID NO:2, 4, 6, 8, 9, 11, 12, 13 or 27, or a fragment of variant thereof) with high affinity. Methods for the production and selection of aptamers having desired specificity are well known to those skilled in the art, and include the SELEX (systematic evolution of ligands by exponential enrichment) process. Briefly, large libraries of oligonucleotides are produced, allowing the isolation of large amounts of functional nucleic acids by an iterative process of in vitro selection and subsequent amplification through polymerase chain reaction. Preferred methodologies for producing aptamers include those disclosed in WO 2004/042083.

Agents, for use according to the invention, may also comprise small molecule inhibitors, which may be identified as part of a high throughput screen of small molecule libraries, as described below.

Accordingly, in a ninth aspect, there is provided a method for identifying an agent that modulates dimerisation or higher order assembly of at least one complement factor H-related (CFHR) protein selected from a group consisting of: CFHR1, CFHR2, CFHR3, CFHR4 and CFHR5, the method comprising:—

(i) contacting, in the presence of a test agent, a first protein selected from a group consisting of: CFHR1, CFHR2, CFHR3, CFHR4 and CFHR5, with a second protein selected from a group consisting of: CFHR1, CFHR2, CFHR3, CFHR4 and CFHR5; and
(ii) detecting binding between the first and second proteins, wherein an alteration in binding as compared to a control is an indicator that the agent modulates dimerisation or higher order assembly of at least one complement factor H-related (CFHR) protein selected from a group consisting of: CFHR1, CFHR2, CFHR3, CFHR4 and CFHR5.

In a tenth aspect, there is provided a method for identifying a candidate agent, for use in the treatment, prevention or amelioration of a disease characterised by inappropriate complement activation, the method comprising the steps of:—

(i) contacting, in the presence of a test agent, a first protein selected from a group consisting of: CFHR1, CFHR2, CFHR3, CFHR4 and CFHR5, with a second protein selected from a group consisting of: CFHR1, CFHR2, CFHR3, CFHR4 and CFHR5; and
(ii) detecting binding between the first and second proteins, wherein an alteration in binding as compared to a control is an indicator that the agent is a candidate for the treatment, prevention of amelioration a disease characterised by inappropriate complement activation.

In an eleventh aspect, there is provided an assay for identifying an agent that modulates dimerisation or higher order assembly of at least one complement factor H-related (CFHR) protein selected from a group consisting of: CFHR1, CFHR2, CFHR3, CFHR4 and CFHR5, the method comprising:—
(i) a first protein selected from a group consisting of: CFHR1, CFHR2, CFHR3, CFHR4 and CFHR5;
(ii) a second protein selected from a group consisting of: CFHR1, CFHR2, CFHR3, CFHR4 and CFHR5; and
(iii) a vessel configured to permit contacting of at least one test agent with the first and/or second agent.

In a twelfth aspect, there is provided the ex vivo use of a colourimetrically- or fluorescentally-labelled first protein selected from a group consisting of: CFHR1, CFHR2, CFHR3, CFHR4 and CFHR5, and/or a colourimetrically- or fluorescentally-labelled second protein selected from a group consisting of: CFHR1, CFHR2, CFHR3, CFHR4 and CFHR5, for identifying an agent which modulates dimerisation or higher order assembly of at least one complement factor H-related (CFHR) protein selected from a group consisting of: CFHR1, CFHR2, CFHR3, CFHR4 and CFHR5.

The first peptide may comprise CFHR1, CFHR2, CFHR3, CFHR4 and/or CFHR5. The second peptide may comprise CFHR1, CFHR2, CFHR3, CFHR4 and/or CFHR5. In embodiments, where the first peptide and the peptide are the same, the method may comprise identifying an agent which modulates homodimerisation. However, when the first peptide and the peptide are different, the method may comprise identifying an agent which modulates heterodimerisation.

In the sections below, the CFHR1:CFHR2 heterodimer interaction is used purely as an example as to how a suitable agent may be identified. A decrease in binding of the first protein to the second protein in the presence of the test agent as compared to a control may be an indicator that the test agent reduces dimerisation between CFHR1 and CFHR2. Conversely, an increase in binding of the first protein to the second protein in the presence of the test agent as compared to a control may be an indicator that the test agent increases dimerisation between CFHR1 and CFHR2. It is preferred that the methods involve identifying an agent that reduces or inhibits dimerisation or higher order assembly.

Any of the methods described herein may be carried out ex vivo. The contacting may be in a substantially cell-free system. Any of the methods may comprise screening an agent that shows a positive indication for the same activity in a cell-based system and/or in vivo in a non-human mammal.

The dimerisation motif may be used as the basis for screens aimed at identifying small molecules (such as antibodies) that specifically disrupt CFHR:CFHR interaction, e.g. by targeting this region of CFHR. Therefore, the first and second peptides used in the methods may each comprise a conserved motif represented by SEQ ID No:12, 13 or 27, or a functional fragment or variant thereof. Accordingly, in certain embodiments, screening systems are contemplated that screen for the ability of test agents to bind these specific residues.

Methods of screening for agents that bind the dimerisation motif of CFHR proteins are readily available to the skilled technician. For example, in one embodiment, the first or second protein is immobilized and probed with test agents. Detection of the test agent (e.g., via a label attached to the test agent) indicates that it binds to the target moiety and is a good candidate modulator of dimerisation. In another embodiment, the dimerisation of CFHR's in the presence of one or more test agents is assayed. This can be accomplished using, for example, a fluorescence resonance energy transfer system (FRET) comprising a donor fluorophore on one moiety (e.g., on the first protein) and an acceptor fluorophore on the second protein. The donor and acceptor quench each other when brought into proximity by the interaction or dimerisation of the first and second proteins. When association is reduced or prevented by a test agent, the FRET signal decreases indicating that the test agent inhibits interaction of the first and second proteins, and that dimerisation is inhibited.

It will be appreciated that agents according to the invention may be used in a medicament which may be used in a monotherapy (i.e. use of only an agent, which reduces the concentration or activity of, or reduces or inhibits dimerisation or higher order assembly of, CFHR1, CFHR2, CFHR3, CFHR4 and/or CFHR5), for treating, ameliorating, or preventing a disease characterised by excessive complement activation. Alternatively, agents according to the invention may be used as an adjunct to, or in combination with, known therapies for treating, ameliorating, or preventing diseases characterised by excessive complement activation.

The agents according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

Medicaments comprising agents according to the invention may be used in a number of ways. For instance, oral administration may be required, in which case the agents may be contained within a composition that may, for example, be ingested orally in the form of a tablet, capsule or liquid. Compositions comprising agents of the invention may be administered by inhalation (e.g. intranasally). Compositions may also be formulated for topical use. For instance, creams or ointments may be applied to the skin.

Agents according to the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with agents used according to the invention is required and which would normally require frequent administration (e.g. at least daily injection).

In a preferred embodiment, agents and compositions according to the invention may be administered to a subject by injection into the blood stream or directly into a site requiring treatment. For example, the medicament may be injected at least adjacent a kidney, if treating nephropathy. Injections may be intravenous (bolus or infusion) or subcutaneous (bolus or infusion), or intradermal (bolus or infusion).

It will be appreciated that the amount of the agent that is required is determined by its biological activity and bioavailability, which in turn depends on the mode of administration, the physiochemical properties of the agent (for example an antibody), and whether it is being used as a monotherapy, or in a combined therapy. The frequency of administration will also be influenced by the half-life of the agent within the subject being treated. Optimal dosages to be administered may be determined by those skilled in the art, and will vary with the particular agent in use, the strength of the pharmaceutical composition, the mode of administration, and the advancement of the cancer, dementia or muscular dystrophy. Additional factors depending on the particular subject being treated will result in a need to adjust dosages, including subject age, weight, gender, diet, and time of administration.

Generally, a daily dose of between 0.01 μg/kg of body weight and 500 mg/kg of body weight of the agent (e.g. an antibody) according to the invention may be used for treating, ameliorating, or preventing cancer, dementia or muscular dystrophy, depending upon which agent is used. More preferably, the daily dose is between 0.01 mg/kg of body weight and 400 mg/kg of body weight, more preferably between 0.1 mg/kg and 200 mg/kg body weight, and most preferably between approximately 1 mg/kg and 100 mg/kg body weight.

The agent may be administered before, during or after onset of the disease to be treated. Daily doses may be given as a single administration (e.g. a single daily injection). Alternatively, the agent may require administration twice or more times during a day. As an example, agents may be administered as two (or more depending upon the severity of the cancer being treated) daily doses of between 25 mg and 7000 mg (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of agents according to the invention to a patient without the need to administer repeated doses.

Known procedures, such as those conventionally employed by the pharmaceutical industry (e.g. in vivo experimentation, clinical trials, etc.), may be used to form specific formulations comprising the agents according to the invention and precise therapeutic regimes (such as daily doses of the agents and the frequency of administration). The inventors believe that they are the first to describe a pharmaceutical composition for treating diseases that are characterised by excessive complement activation, based on the use of the agents and inhibitors of the invention.

Hence, in a thirteenth aspect of the invention, there is provided a pharmaceutical composition, comprising an agent which:

(i) reduces the concentration or activity of at least one complement factor H-related (CFHR) protein selected from a group consisting of: CFHR1, CFHR2, CFHR3, CFHR4 and CFHR5; or
(ii) reduces or inhibits dimerisation or higher order assembly of at least one CFHR protein selected from a group consisting of: CFHR1, CFHR2, CFHR3, CFHR4 and CFHR5, and a pharmaceutically acceptable vehicle.

The composition can be used in the therapeutic amelioration, prevention or treatment of any disease in a subject caused by excessive complement activation. Examples of such diseases are provided herein. Therefore, for example only, the composition may be age-related macular degeneration (AMD) treatment composition, a meningitis treatment composition, a renal disease (e.g. C3 glomerulopathy) treatment composition, an arthritis treatment composition, or an autoimmune disease or inflammation treatment composition.

Preferably, the agent comprises an antibody or antigen binding fragment thereof.

The invention also provides in an fourteenth aspect, a process for making the pharmaceutical composition according to the thirteenth aspect, the process comprising contacting a therapeutically effective amount of an agent which:

(i) reduces the concentration or activity of at least one complement factor H-related (CFHR) protein selected from a group consisting of: CFHR1, CFHR2, CFHR3, CFHR4 and CFHR5; or
(ii) reduces or inhibits dimerisation or higher order assembly of at least one CFHR protein selected from a group consisting of: CFHR1, CFHR2, CFHR3, CFHR4 and CFHR5, and a pharmaceutically acceptable vehicle.

The agent may comprise an antibody.

A “subject” may be a vertebrate, mammal, or domestic animal. Hence, agents, compositions and medicaments according to the invention may be used to treat any mammal, for example livestock (e.g. a horse), pets, or may be used in other veterinary applications. Most preferably, however, the subject is a human being.

A “therapeutically effective amount” of agent is any amount which, when administered to a subject, is the amount of drug that is needed to treat the target disease, or produce the desired effect, i.e. increasing CFH activity or decreasing complement activation.

For example, the therapeutically effective amount of agent used may be from about 0.01 mg to about 800 mg, and preferably from about 0.01 mg to about 500 mg. It is preferred that the amount of agent is an amount from about 0.1 mg to about 250 mg, and most preferably from about 0.1 mg to about 20 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent (e.g. the siRNA molecule, peptide or antibody) may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The active agent according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The agent may be prepared as a sterile solid composition that may be dissolved or suspended at the time of administration using sterile water, saline, or other appropriate sterile injectable medium.

The agents and compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The agents used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

It will be appreciated that the invention extends to any nucleic acid or peptide or variant, derivative or analogue thereof, which comprises substantially the amino acid or nucleic acid sequences of any of the sequences referred to herein, including functional variants or functional fragments thereof. The terms “substantially the amino acid/nucleotide/peptide sequence”, “functional variant” and “functional fragment”, can be a sequence that has at least 40% sequence identity with the amino acid/nucleotide/peptide sequences of any one of the sequences referred to herein, for example 40% identity with the sequence identified as SEQ ID No: 1, 3, 5, 7 or 10 (i.e. CFHR1-5 gene), or 40% identity with the polypeptide identified as SEQ ID No: 2, 4, 6, 8, 9 or 11 (i.e. the CFHR1-5 protein), and so on.

Amino acid/polynucleotide/polypeptide sequences with a sequence identity which is greater than 50%, more preferably greater than 65%, 70%, 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90%, 92%, 95%, 97%, 98%, and most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps but excluding overhangs. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to any sequences referred to herein or their complements under stringent conditions. By stringent conditions, we mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in SEQ ID No: 2, 4, 6, 8, 9 or 11.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:—

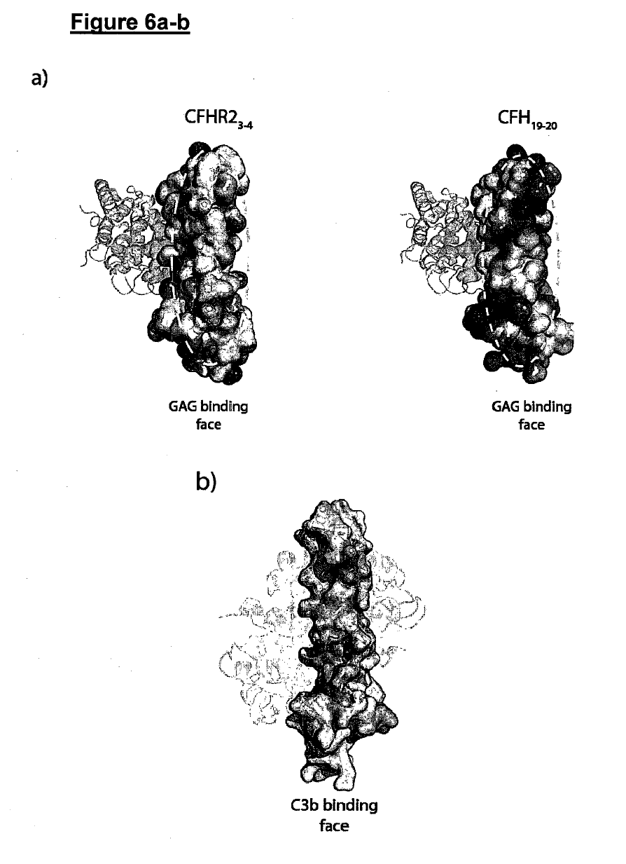

FIG. 1 shows that CFHR1, CFHR2 and CFHR5 contain an identical novel dimerization motif. FIG. 1(a) Alignment of the SCR domains of CFHR1, CFHR2 and CFHR5 with CFH. These proteins are comprised of subunits termed short consensus repeat (SCR) domains and domains have been aligned according to the CFH domain with which they share the highest amino acid similarity, percentage identity indicated. Red boxing denotes domains for which novel X-ray structures are presented in this manuscript. The complement regulatory domains of CFH reside within the first four amino-terminal domains (cyan). None of the CFHR proteins contain domains similar to these. CFH surface recognition domains which contain C3b/C3d and glycosaminoglycan (GAG) binding sites reside within the carboxyl-terminal two domains (CFH₁₉₋₂₀) and all three CFHR proteins contain highly similar domains. Mapping of the conserved residues onto the existing structure of CFH₁₉₋₂₀ suggests that GAG but not C3b/C3d binding is altered or lost within CFHR2₃₋₄ (see FIGS. 6 & 7). The first two amino-terminal domains of CFHR1, CFHR2 and CFHR5 are highly conserved and have previously been described as CFH₆₇-like, although the level of identity is less than 40%. FIG. 1(b) X-ray crystal structure of CFHR1₁₋₂. The two copies of CFHR1₁₂ that form the head-to-tail dimer are shown as grey cartoons with a semi-transparent surface. Residues Tyr34, Ser36 and Tyr39 that are critical in stabilising the dimer are shown in a ball-and-stick representation (Figure drawn using program PyMol, www.pymol.org). FIG. 1(c) Sequence alignment of CFHR1₁₋₂, CFHR2₁₋₂ CFHR5₁₋₂ with CFH₆₋₇. The dimerization interface is conserved between these CFHR proteins but not in CFH. (interface residues determined using PISA; residues Tyr34, Ser36 and Tyr39 indicated by *; other interface residues by •). Red boxed residues=non-conservative, green boxed residues=conservative variation and yellow boxed residues=residues unique to CFH₆₇. FIG. 1(d) Mapping sequence variation onto the molecular surface of one copy of CFHR1₁₂. This analysis confirmed that the dimerization interface is conserved amongst CFHR1₁₂, CFHR2₁₂ and CFHR5₁₂ but not in CFH₆₇ (positions of Tyr34, Ser36 and Tyr 39 indicated with *);

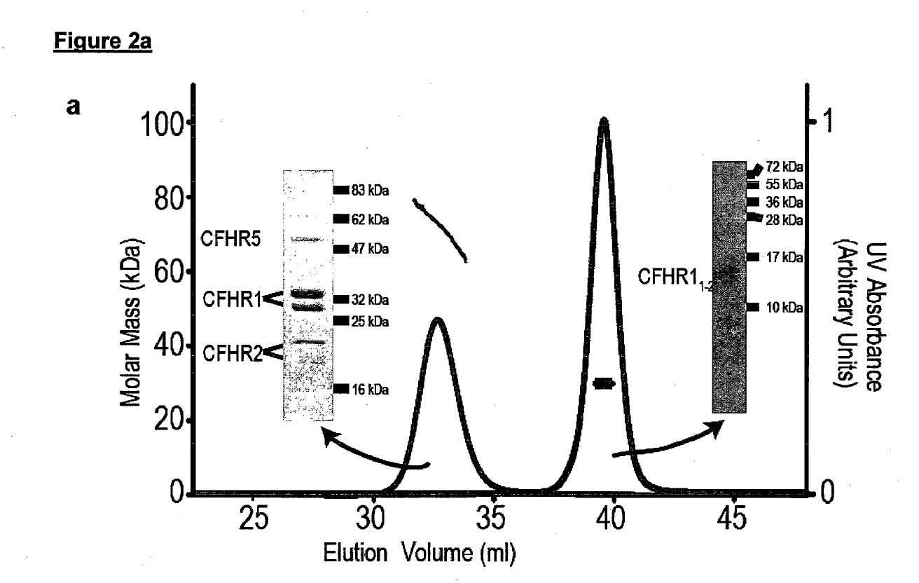

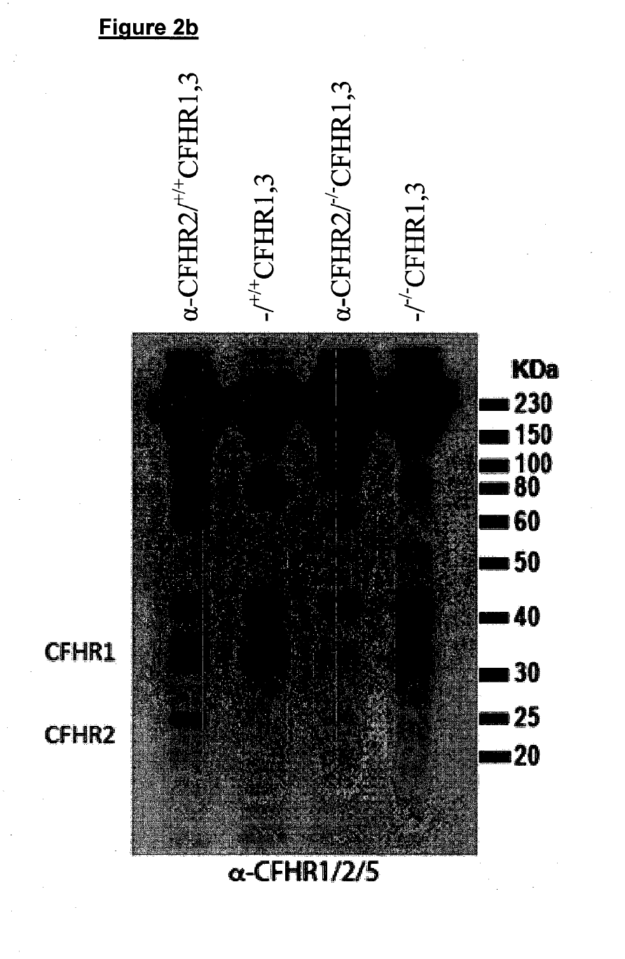

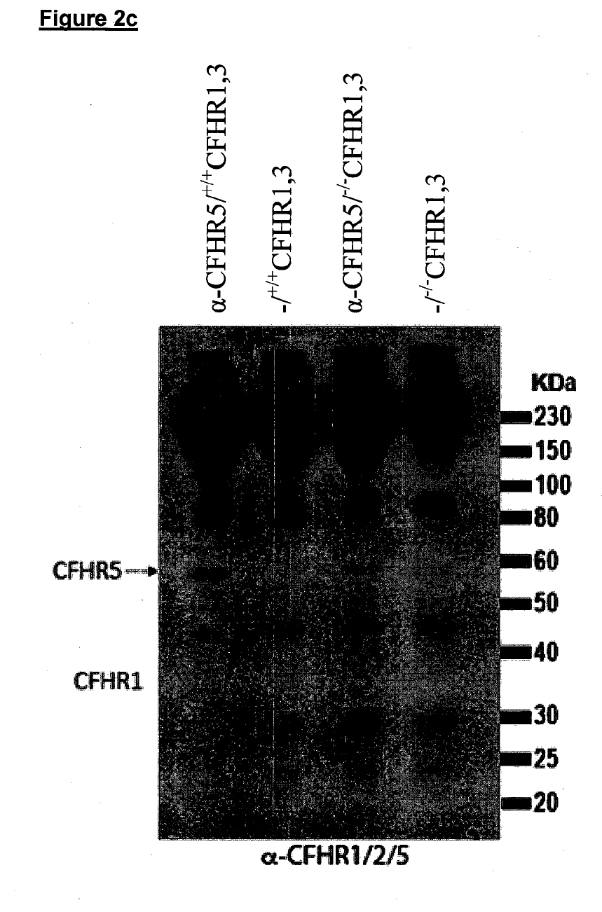

FIG. 2 shows that CFHR1, CFHR2 and CFHR5 are dimeric in serum. FIG. 2(a) Multi-angle light scattering analyses (MALS) of a (i) serum fraction containing CFHR1, CFHR2 and CFHR5 and (ii) recombinant CFHR1₁₋₂. MALS analysis of this fraction (red) demonstrates that this mixture contains a mass range between 65 and 80 kDa. MALS using recombinant CFHR1₁₋₂ (blue trace and mass profile) demonstrates that it forms a homogenous dimer in both solution and crystal. FIG. 2 (b) Immunoprecipitation of CFHR2 in serum reveals the presence of CFHR1-CFHR2 heterodimers in vivo. Serum was immunoprecipitated using a specific anti-CFHR2 antibody (MBC22) and western blot analysis of the immunoprecipitated material with anti-CFHR1/2/5 antibody (MBC125) performed. This revealed the presence of CFHR1 (lane 1) which was absent in serum from an individual homozygous for the ΔCFHR3-1 deletion polymorphism (lane 3). Lane 2 and 4 represent control sera in which no anti-CFHR2 antibody was used. The detection of CFHR2-CFHR5 heterodimers was not possible due to non-specific bands in the CFHR5 region. FIG. 2(c) Immunoprecipitation of CFHR5 in serum reveals the presence of CFHR1-CFHR5 heterodimers in vivo. Serum was immunoprecipitated using an anti-CFHR5 antibody and western blot analysis of the immunoprecipitated material with anti-CFHR1/2/5 antibody performed. This revealed the presence of CFHR1 (lane 1) which was absent in serum from an individual homozygous for the ΔCFHR3-1 deletion polymorphism (lane 3). Lane 2 and 4 represent control sera in which no anti-CFHR5 antibody was used. The detection of CFHR2-CFHR5 heterodimers was not possible due to non-specific bands in the CFHR2 region. FIG. 2(d) ELISA assay to detect CFHR2-CFHR5 heterodimers in vivo. Using an anti-CFHR5 capture antibody and an anti-CFHR2 detection antibody, positive signal was demonstrable in two individuals homozygous for the ΔCFHR3-1 deletion polymorphism. A much weaker signal was detectable in individuals without this deletion. No signal was seen when recombinant CFHR5 was tested indicating that the anti-CFHR2 detection antibody does not cross-react with CFHR5;

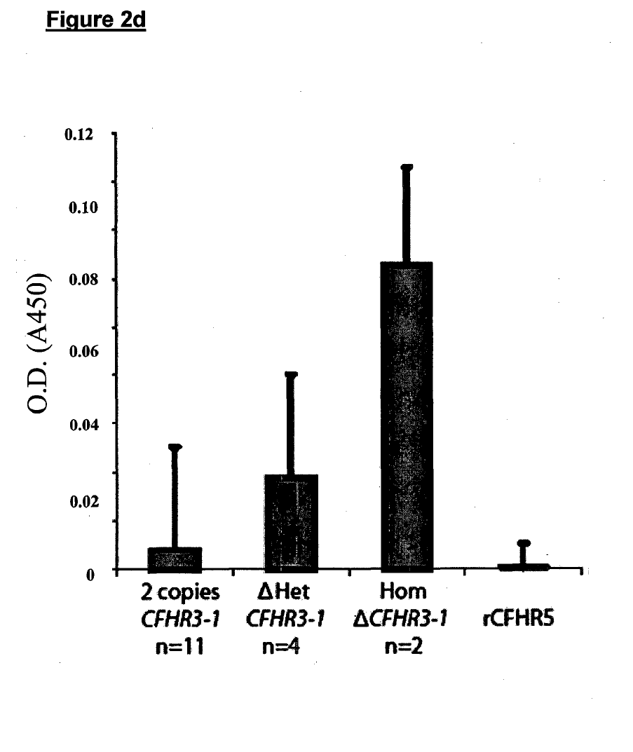

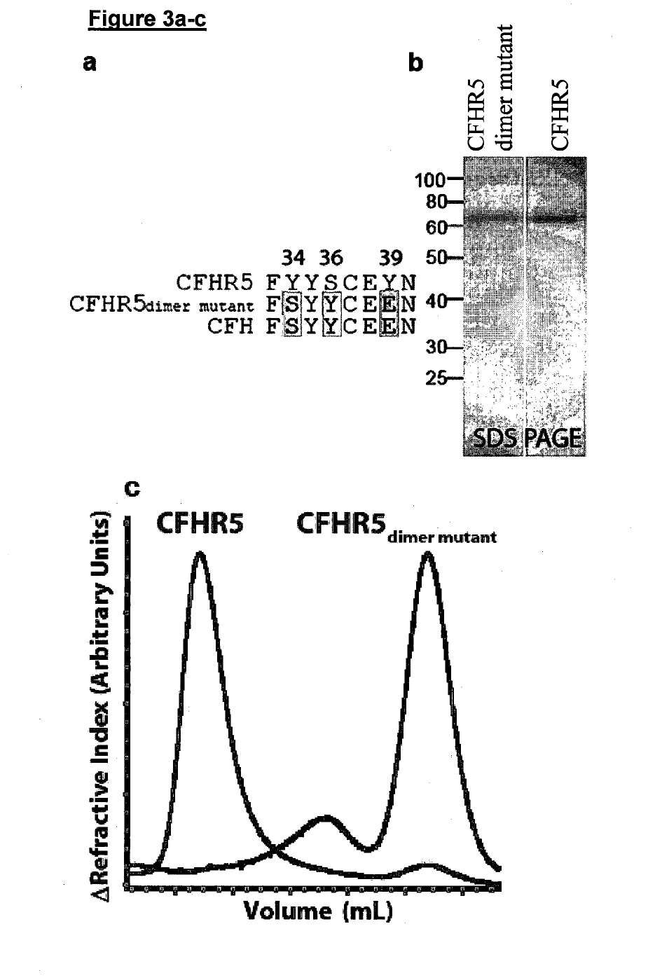

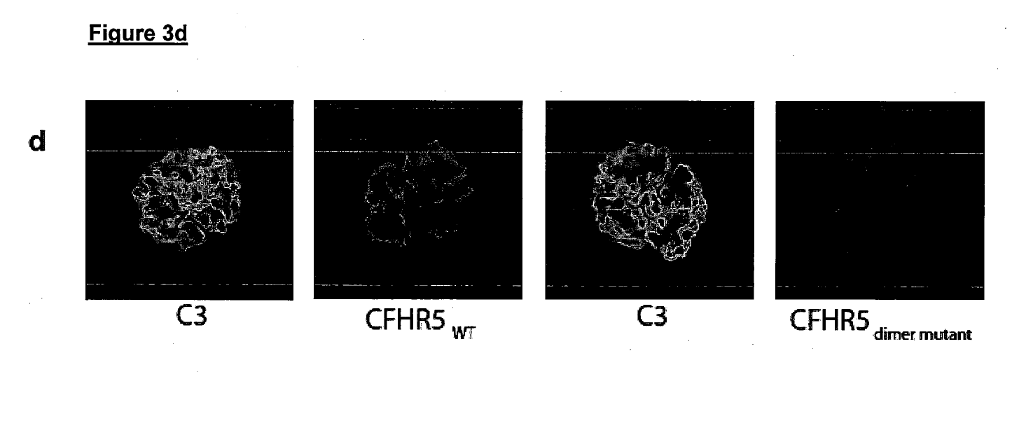

FIG. 3 shows that dimerisation enhances the interaction of CFHR5 with complement c3 in vivo. FIG. 3(a) Generation of a CFHR5 protein lacking critical amino acids within the dimerisation motif. Monomeric CFHR5 (CFHR5^{dimer mutant}) was generated by mutating the three stabilizing amino acids (Tyr34Ser, Ser36 Tyr, Tyr39Glu) within the dimerisation motif to the corresponding amino acids within CFH. FIG. 3(b) Analysis of recombinant CFHR5 and CFHR5^{dimer mutant} using SDS PAGE gel electrophoresis. Both the wild type and dimer mutants were purified to single homogenous species as visualized by denaturing electrophoresis. FIG. 3(c) Analysis of recombinant CFHR5 and CFHR5^{dimer mutant} using size exclusion chromatography. Size exclusion chromatography was performed on a Superdex200 10/30 column (GE Healthcare) equilibrated in 50 mM Tris.HCl, pH 7.5, 150 mM NaCl at 0.4 ml/min. The column was followed in-line by an Optilab-Rex refractive index monitor (Wyatt Technologies). The CFHR5 dimer elutes early from the column (blue trace) whilst the monomeric CFHR5^{dimer mutant} protein elutes at a larger column volume (red trace). FIG. 3(d) Interaction of CFHR5 and CFHR5^{dimer mutant} with renal-bound mouse C3 in vivo. When recombinant CFHR5^{dimer mutant} was injected at identical concentration to that of CFHR5, CFHR5^{dimer mutant} binding to glomerular C3 was significantly reduced compared to that of wild-type CFHR5;

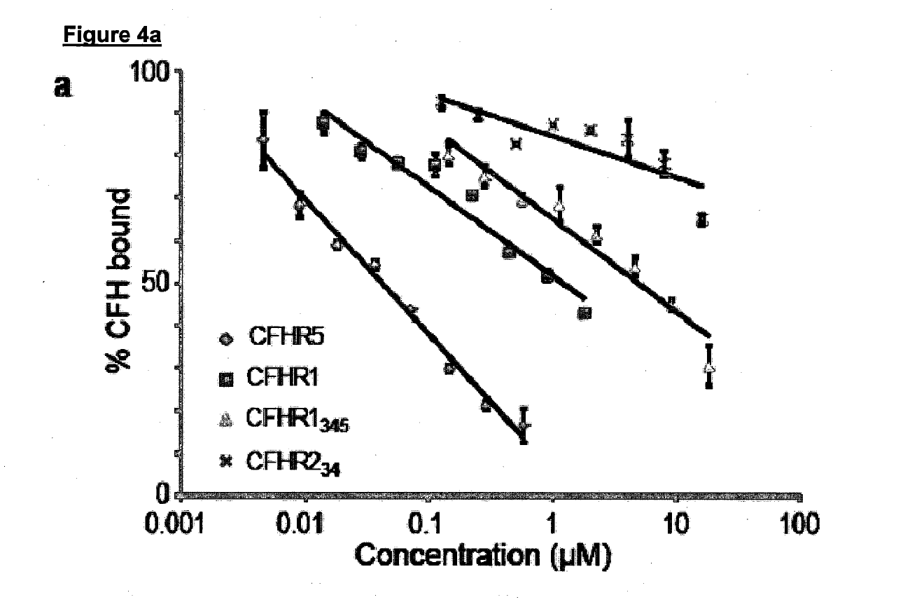

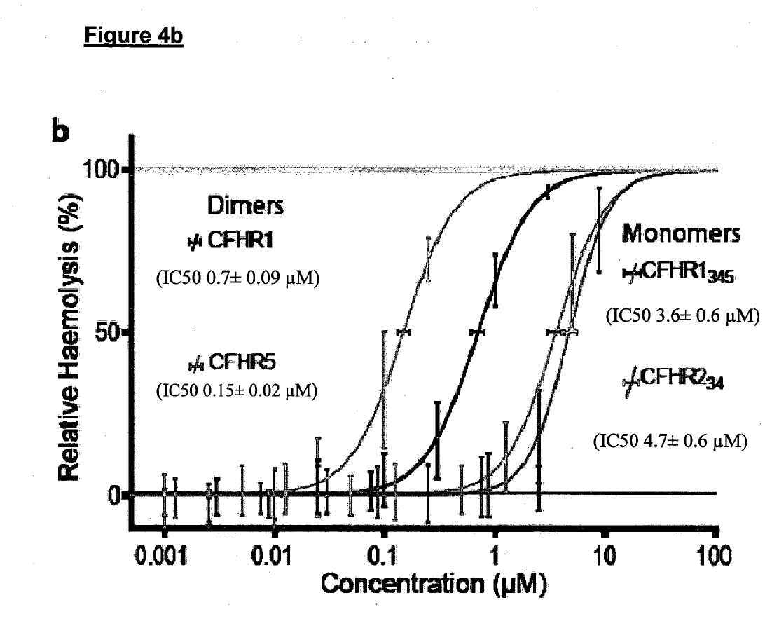

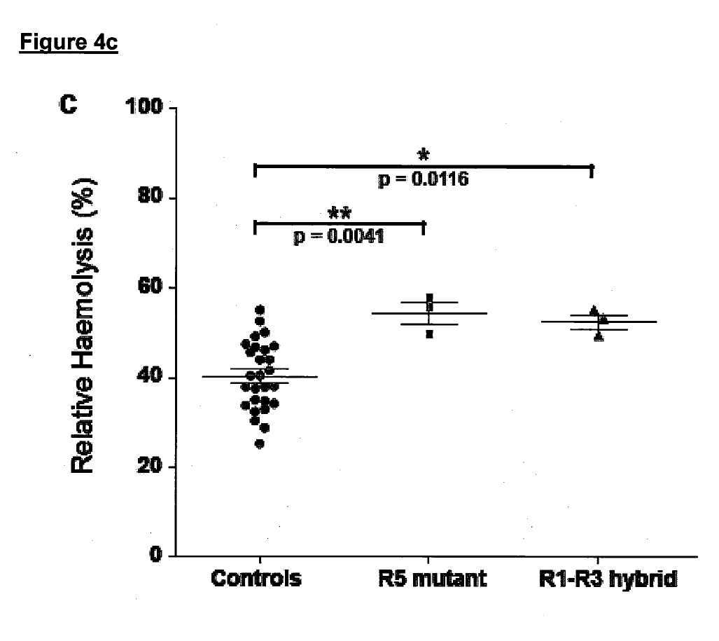

FIG. 4 shows that CFHR1 and CHFR5 de-regulate complement activation by competitively inhibiting the interaction of cfh with cab. FIG. 4(a) CFH binding to C3b is inhibited by either recombinant CFHR5 or serum-derived CFHR1. ELISA wells were coated with C3b and 0.07 μM CFH was incubated with increasing amounts of either CFHR1 (0.014 to 1.8 μM) or CFHR5 (0.005 to 0.6 μM). Both proteins reduced the CFH-C3b interaction in a dose-dependent manner. Similar results were obtained when recombinant CFHR1₃₄₅ (0.14 to 18 μM) and CFHR2₃₄ (0.13 to 16 μM) were used. FIG. 4(b) CFH-dependent alternative pathway haemolytic assay. Using a CFH dose that reduced lysis of Guinea-Pig erythrocytes to 50%, the addition of increasing concentrations of CFHR1₃₅, CFHR2₃₄, serum-derived CFHR1 and recombinant CFHR5 resulted in a dose-dependent increase in lysis. Full length, dimeric, CFHR1 and CFHR5 were orders of magnitude more potent with respect to the monomeric CFHR1 and CFHR2 fragments lacking the dimerisation motif. FIG. 4(c) Enhanced de-regulation by plasma-derived preparations containing CFHR1, CFHR2 and CFHR5 from individuals with familial C3 glomerulopathy due to a CFHR5 mutation. Using the haemolytic assay described in (b) serum-derived preparations from patients with CFHR5 mutation associated with C3 glomerulopathy showed significantly greater haemolysis than controls;

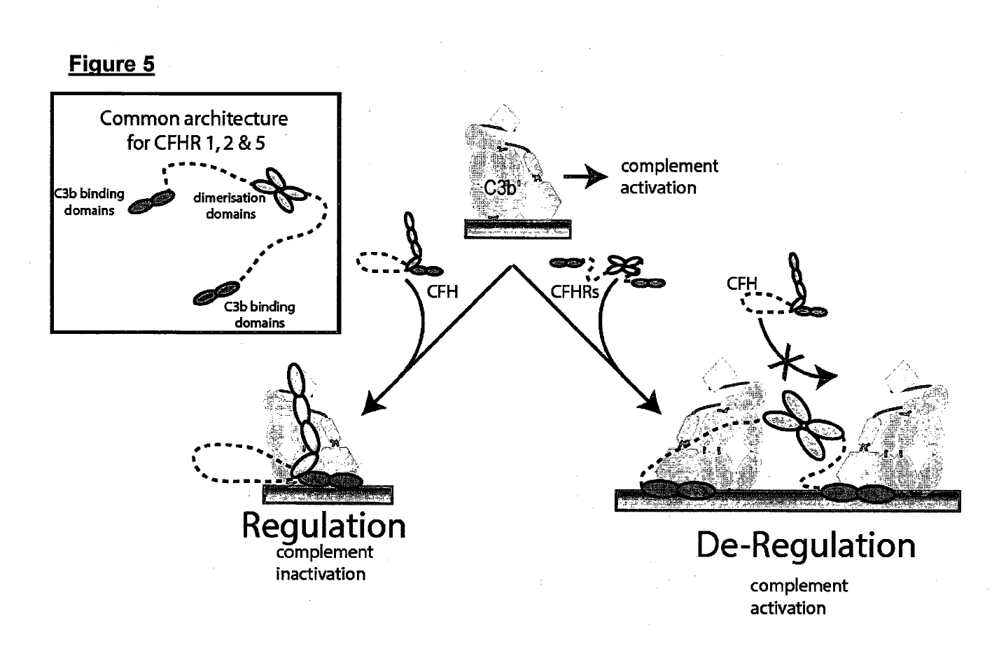

FIG. 5 shows that modulation of complement in vivo by CFHR1, CFHR2 and CFHR5. These proteins compete with CFH for interaction with C3b. Unlike CFH, they are devoid of intrinsic complement regulatory activity under physiological conditions. However, their interaction with C3b prevents the binding of C3b to CFH and thereby prevents inactivation of C3b by CFH. This process is termed “de-regulation”. Whether or not C3b interacts with CFH or components of the CFHR family will be influenced by factors such as C3b density, surface polyanions and the local concentrations of CFH and CFHR proteins. In this way, CFHR proteins provide a sophisticated means through which complement activation can be modulated in vivo. Inset: A general schematic for the functionally important portions of CFHR1, CFHR2 and CFHR5 is shown;

FIG. 6 shows that the C3b interface is conserved in the C-terminal domains but not the GAG binding surface. FIG. 6(a) Crystal structure of CFHR234 suggests GAG binding is altered or lost. The electrostatic potential (contoured at +3 kT/e—blue, −3 kT/e—red; calculated using the APBS plugin within Pymol: www.pymol.org) is mapped onto the surface and that of CFH19-20, PDB 3OXU, shown for comparison. CFH charged GAG-binding surface is ablated (right image, GAG binding surface=yellow dashed outline). FIG. 6(b) Crystal structure of CFHR234 suggests C3b binding maintained despite sequence variation. Sequence variation (identical residues—grey; variation—yellow) between CFHR2 and CFH (PDB 3OXU) mapped onto the CFHR234 structure shows high conservation of the C3b binding site despite the relatively low level of amino acid conservation in these domains between these proteins;

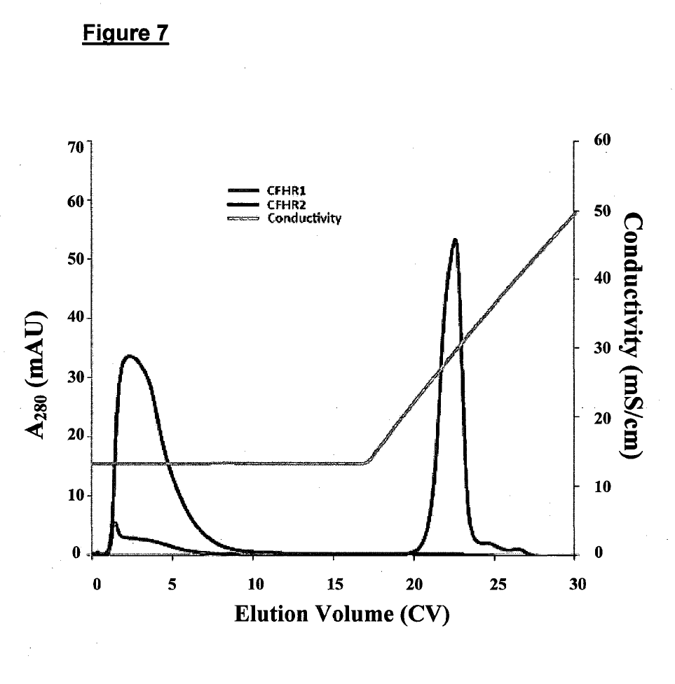

FIG. 7 shows that CFHR1 interacts with heparin via its C-terminal domains (domains 3-5) but CFHR2 does not. Approximately 0.5 mg CFHR1345 and CFHR234 in 50 mM Tris, 10 mM NaCl, pH 7.5 was loaded onto a 1 ml HiTrap Heparin column (GE Healthcare) using an AKTAfplc (GE healthcare). Non-bound material was washed out with 5CV 50 mM Tris, 10 mM NaCl, pH 7.5 prior to a gradient elution of 50% 50 mM Tris, 1M NaCl, pH 7.5 over 15CV. CFHR234 did not bind and was washed out during wash step. FHR1345 eluted at 29.6 mS/cm;

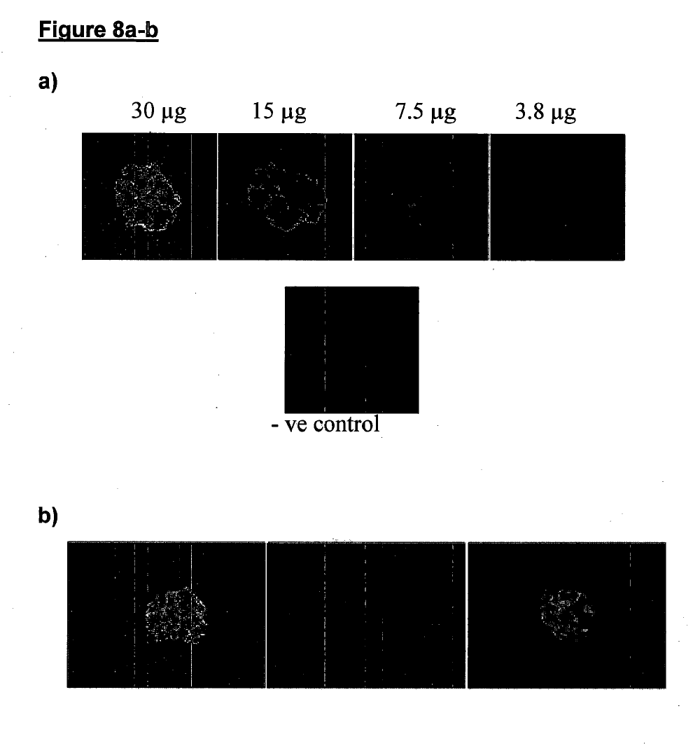

FIG. 8 shows that binding of CFHR5 to C3 in vivo is dose-dependent and targets CFHR5 to the kidney. FIG. 8(a) Binding of CFHR5 to C3 in vivo is dose-dependent. Glomerular CFHR5 staining was reduced when decreased doses of CFHR5 (30, 15, 7.5 and 3.8 μg) were injected into CFH−/− mice. No staining was observed in mice injected with PBS (negative control). FIG. 8(b) Targeting of CFHR5 to the kidney is dependent on C3. Ex vivo binding of CFHR5 to kidney sections of CFH−/− mice and animals with combined deficiency of either Cfh and C3 (CFH.C3−/−), or CFH and C5 (CfH.C5−/−). Glomerular CFHR5 staining was evident only in the presence of C3;

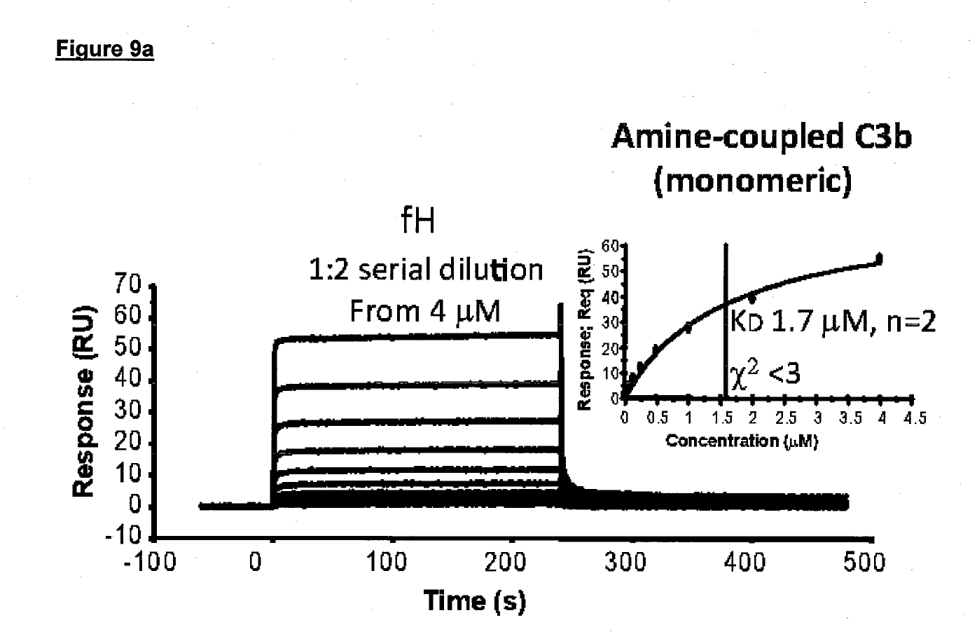

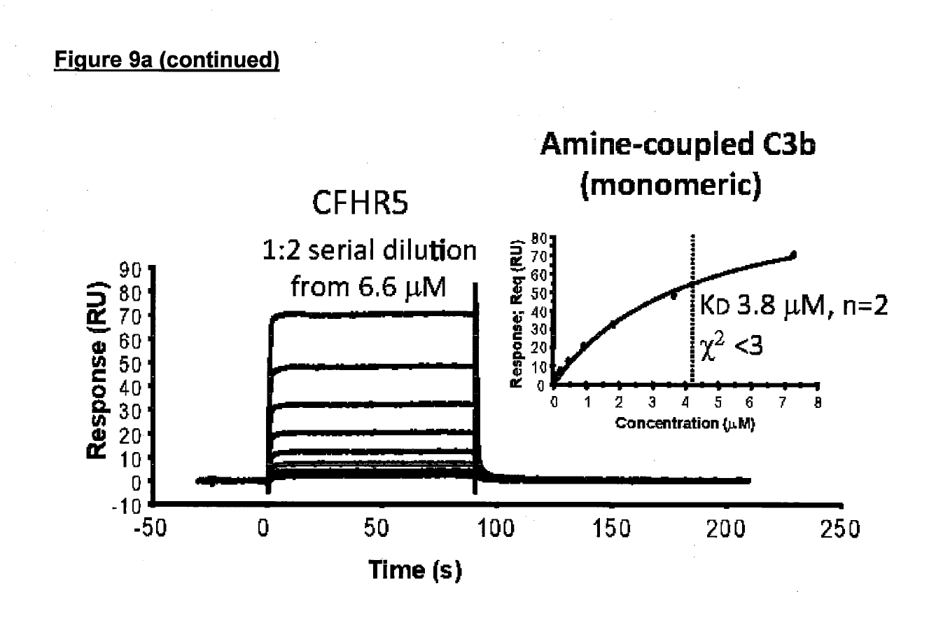

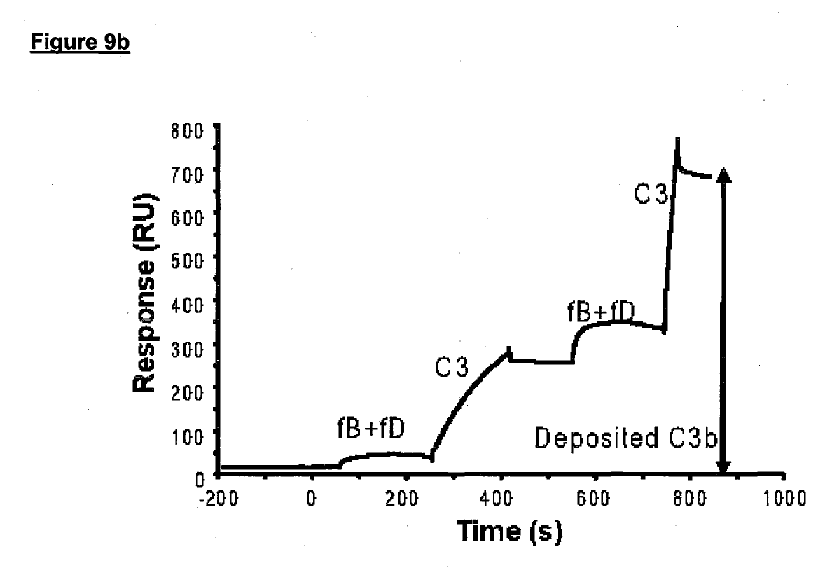

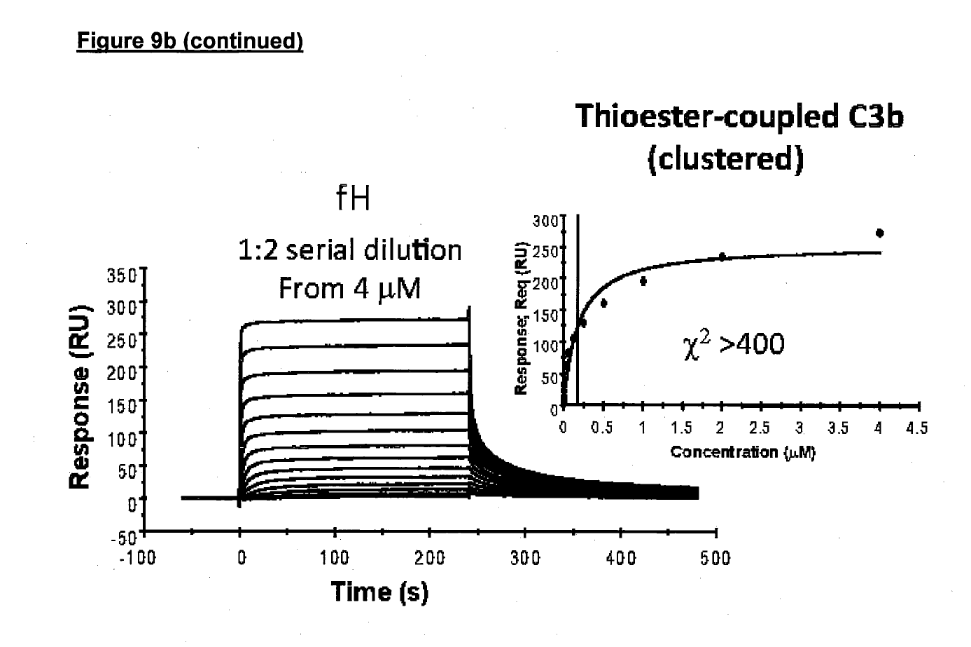

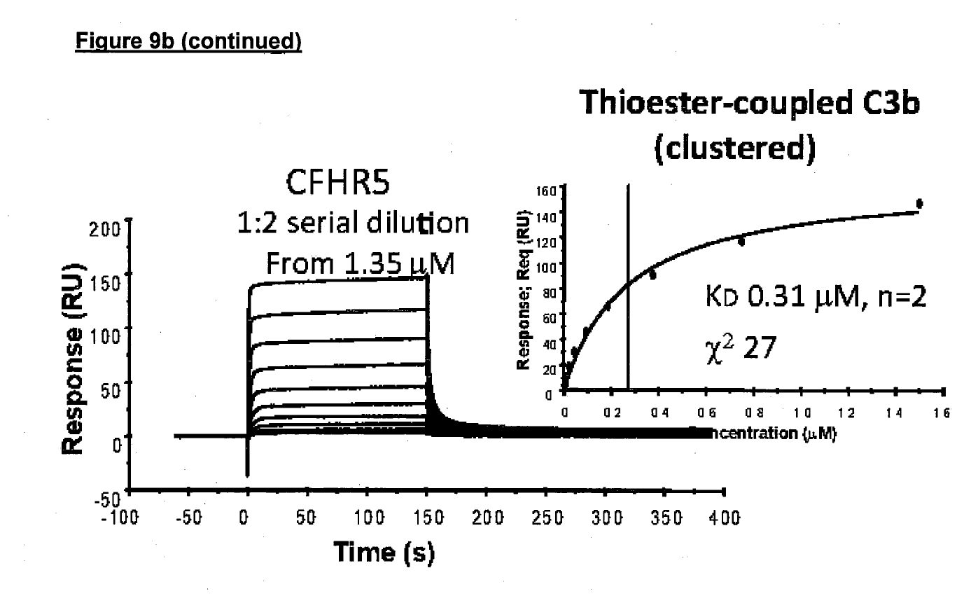

FIG. 9 shows that Surface Plasmon Resonance (SPR) analysis of CFHR5 and CFH binding to C3b. FIG. 9(a) Binding of CFHR5 and CFH to low levels of C3b coupled through amine groups (no clustering of C3b). CFH (from 4 μM) or CFHR5 (from 6.6 μM) were flowed across immobilized C3b (400 RU) at different concentrations. Affinity was calculated by steady state analysis. Analysis assumes a 1:1 binding interaction and for CFHR5 calculations used molarity of binding sites. FIG. 9(b) Binding to C3b coupled through the thiolester. C3b (150RU) was amine-coupled to a CM5 Biacore chip and used as a nidus for convertase formation. Further C3b was deposited on the chip surface by flowing fB and fD to form C3bBb followed by C3 as convertase substrate. Cleavage of C3 to C3b followed by nucleophilic attack on the C3 thioester by CM groups on the chip surface resulted in covalent binding of C3b (625 RU). CFH (from 4 μM) and CFHR5 (from 1.35 μM) were flowed across the surface and binding was analysed at steady state. Binding of CFH was very heterogeneous, likely due to crosslinking between multiple C3b-binding sites on fH and clusters of deposited C3b molecules. Affinity could not be calculated under these conditions. CFHR5 bound to this surface 10-fold more tightly than to the amine-coupled C3b, although binding heterogeneity was increased (see 2nd value). Comparison of (a) and (b) reveals differences in the binding caused by avidity; when C3b is clustered on the chip surface (b), multiple C3b-binding domains within one molecule of CFH or within the CFHR5 dimer can bind and cross-link C3b. Data were calculated using the following values: CFH, mass 155 kDa, extinction coefficient 1.95 cm-1 (mg/mL)-1; CFHR5, mass 65 kDa, extinction coefficient 1.55 cm-1 (mg/mL)-1;

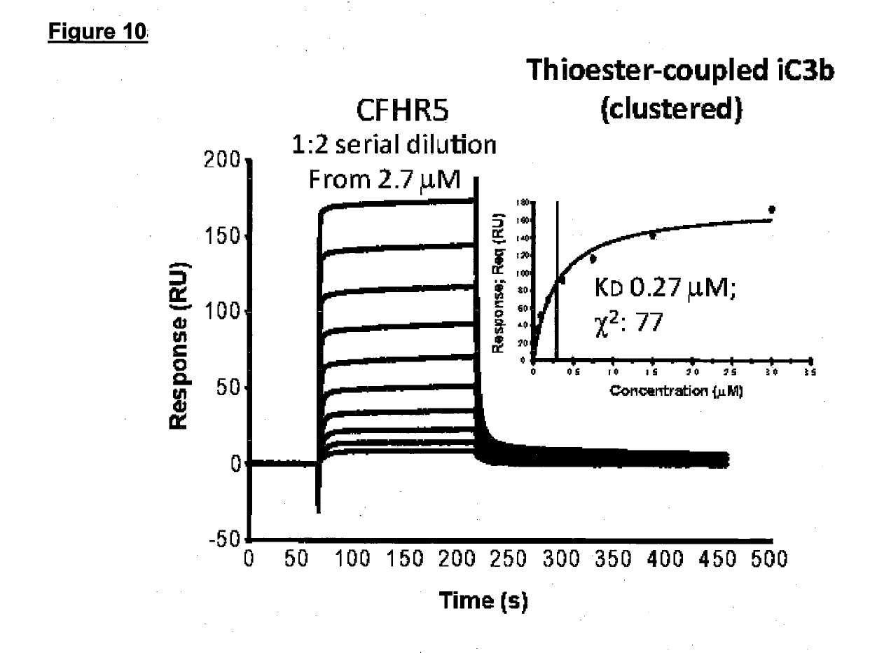

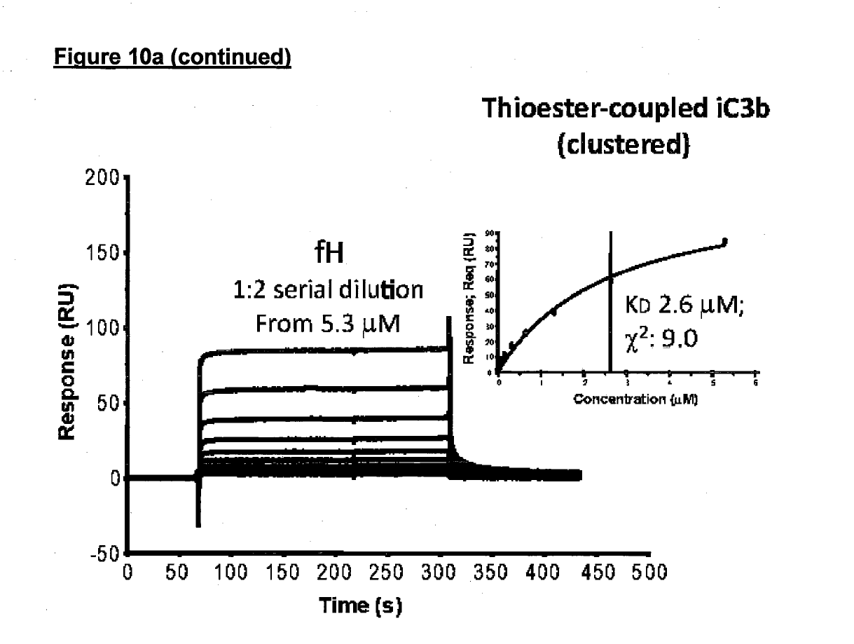

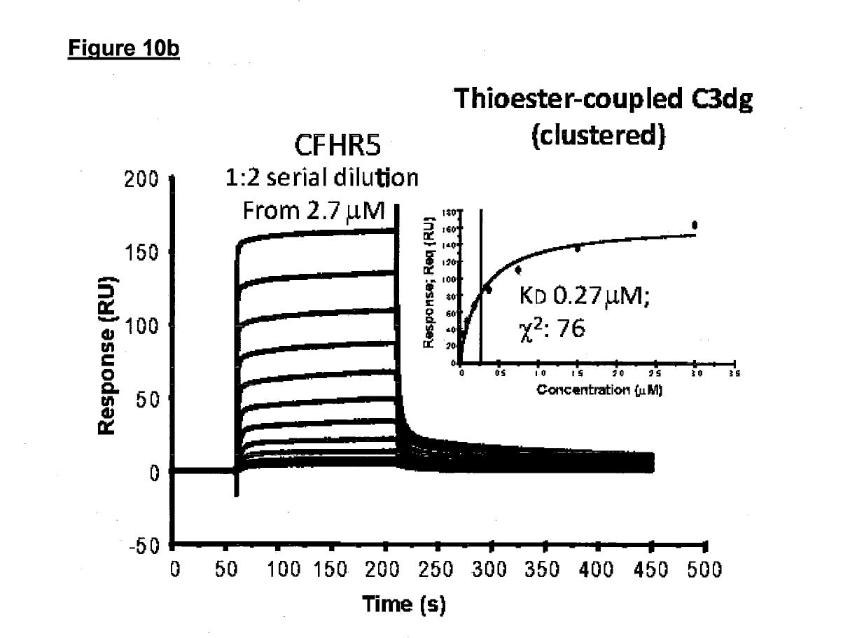

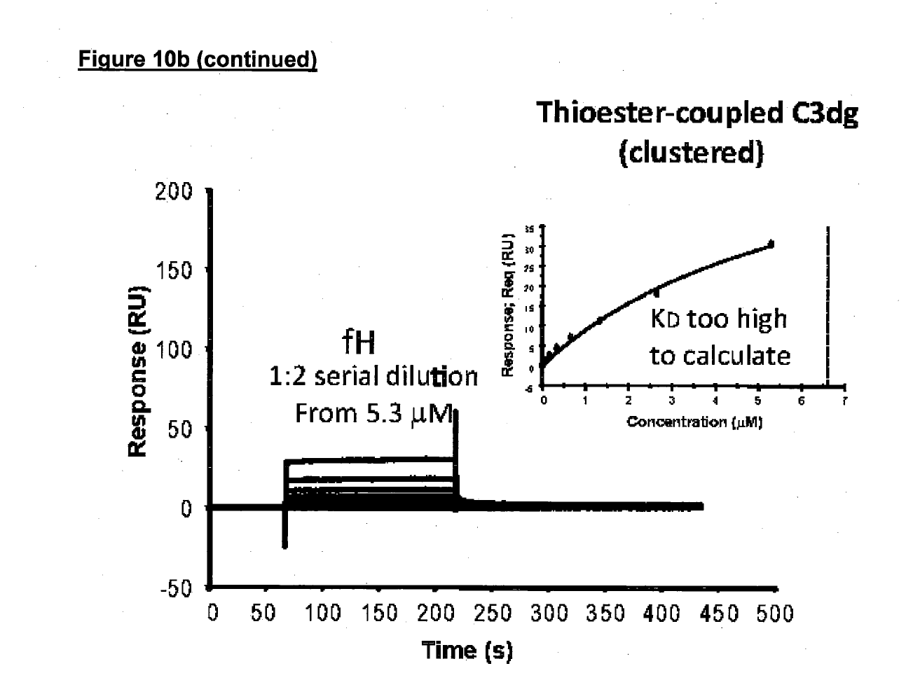

FIG. 10 shows that surface plasmon resonance analysis of CFHR5 and CFH binding to the inactivation fragments, iC3b and C3dg. FIG. 10(a) C3b deposited in FIG. 7(b) was converted to iC3b by on-chip incubation with fH (30 mg/me and fI (10 mg/ml). Binding of CFH (from 5.3 μM) and CFHR5 (from 2.7 μM) to iC3b was assessed by flowing across the surface and evaluating at steady state. Binding of CFHR5 to iC3b was comparable to C3b (although more heterogeneous); binding of fH was vastly reduced compared to C3b and was 10-fold weaker than CFHR5. FIG. 10(b) Binding of CFHR5 and CFH to C3dg coupled through the thiolester was assessed by treating the iC3b surface with CR1 and fI to convert to C3dg, C3c was released from the chip surface. CFH (from 5.3 μM) and CFHR5 (from 2.7 μM) were flowed across the surface and binding evaluated at steady state. Binding of CFHR5 to C3dg was comparable to iC3b and C3b; binding affinity of fH was very weak and could not be calculated under these concentrations. Data were calculated using the following values: CFH, mass 155 kDa, extinction coefficient 1.95 cm-1(mg/mL)-1; CFHR5, mass 65 kDa, extinction coefficient 1.55 cm-1(mg/mL)-1;

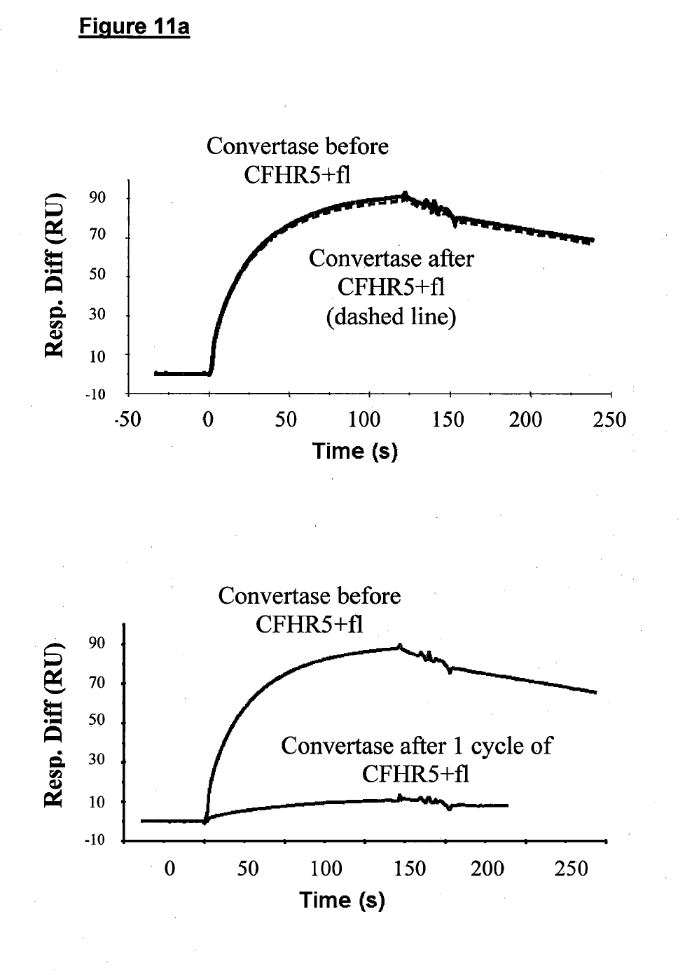

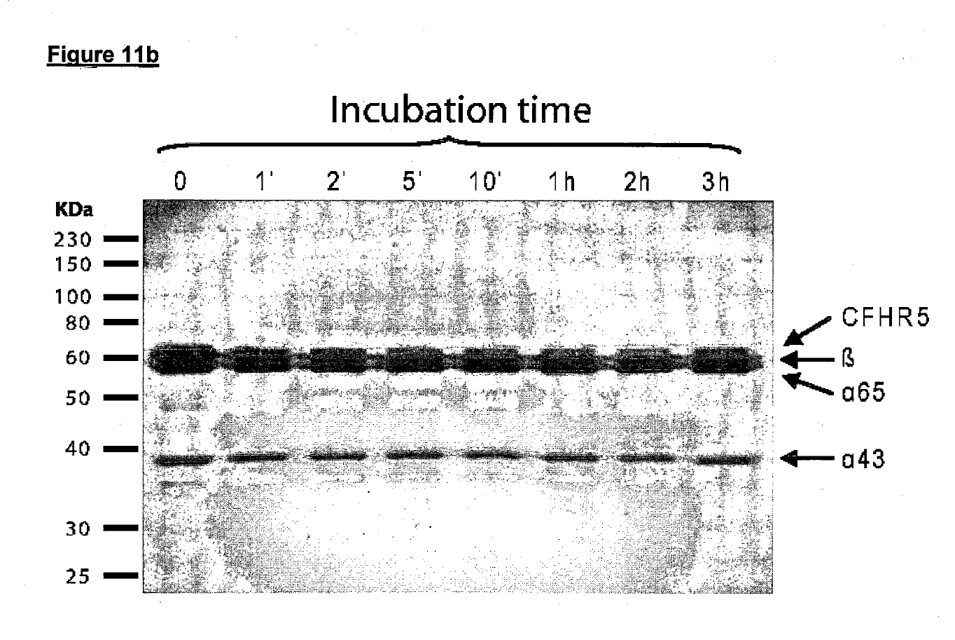

FIG. 11 shows that CFHR5 does not have fluid-phase factor I (fI) cofactor activity for the proteolytic inactivation of either C3b (a) or iC3b (b). (a) C3b was deposited on the chip surface and convertase formation monitored by flowing CFB and FD (left panel, solid line). The surface was then treated twice with CFHR5 (first cycle 0.18 μM and second cycle 0.44 μM) and FI (10 μg/ml constant) for 120 seconds each cycle. Convertase was formed by flowing CFB and FD exactly as before (left panel, dashed line). The amount of convertase formed was identical before and after treatment. In contrast, treatment of the surface with CFH (0.1 μM) and fI ablated convertase formation (right panel). Moreover, no cleavage of the α65 chain of iC3b was observed after the incubation of iC3b (2 μg) with CFHR5 (0.42 mg) and fI (0.12 μg) at 37° C. (b);

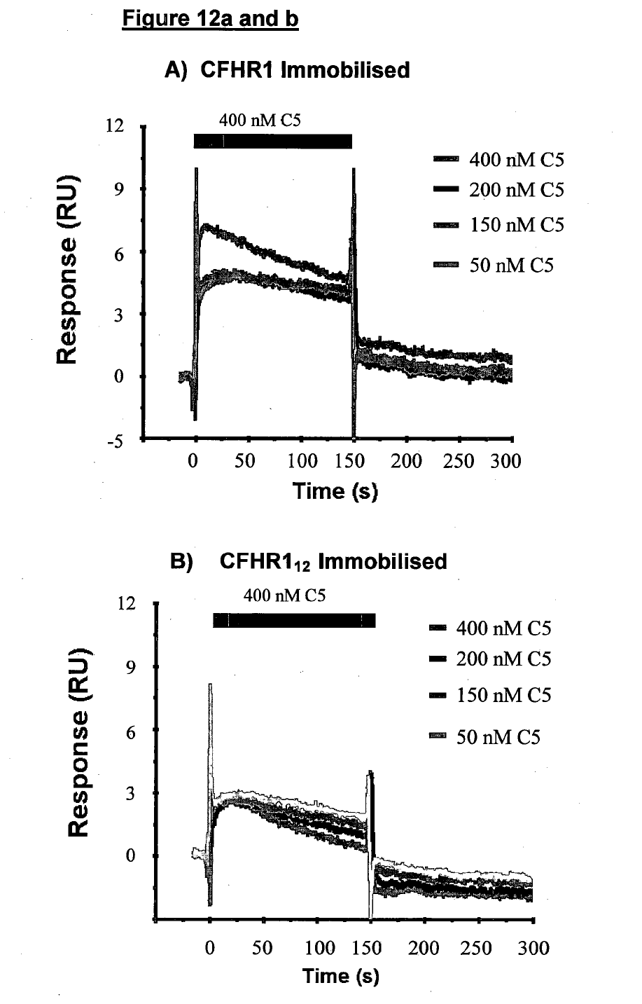

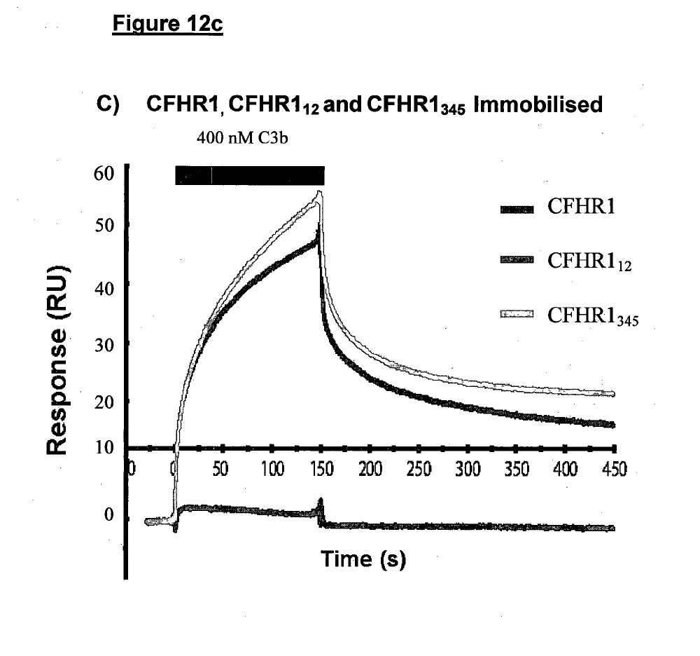

FIG. 12 shows that CFHR1 interacts with C3b and not C5 via its C-terminal domains (domains 3-5). (a) CFHR1 purified from serum or (b) recombinant CFHR1 domains 1 and 2 are immobilised on the sensor chip surface via primary amine coupling (CFHR1-2300RU; CFHR112-750RU) and C5 at concentrations between 50 and 400 nM is flowed across. No significant interaction is seen at any concentration. (c) 400 nM C3b is flown over surfaces with either serum-purified CFHR1, recombinant N-terminal CFHR112 or recombinant C-terminal CFHR1345 (CFHR1-2300RU; CFHR112-750RU; CFHR1345-1800RU). C3b interacts only with the full-length or C-terminal fragments. (Flow rate 20 μl/min all panels);

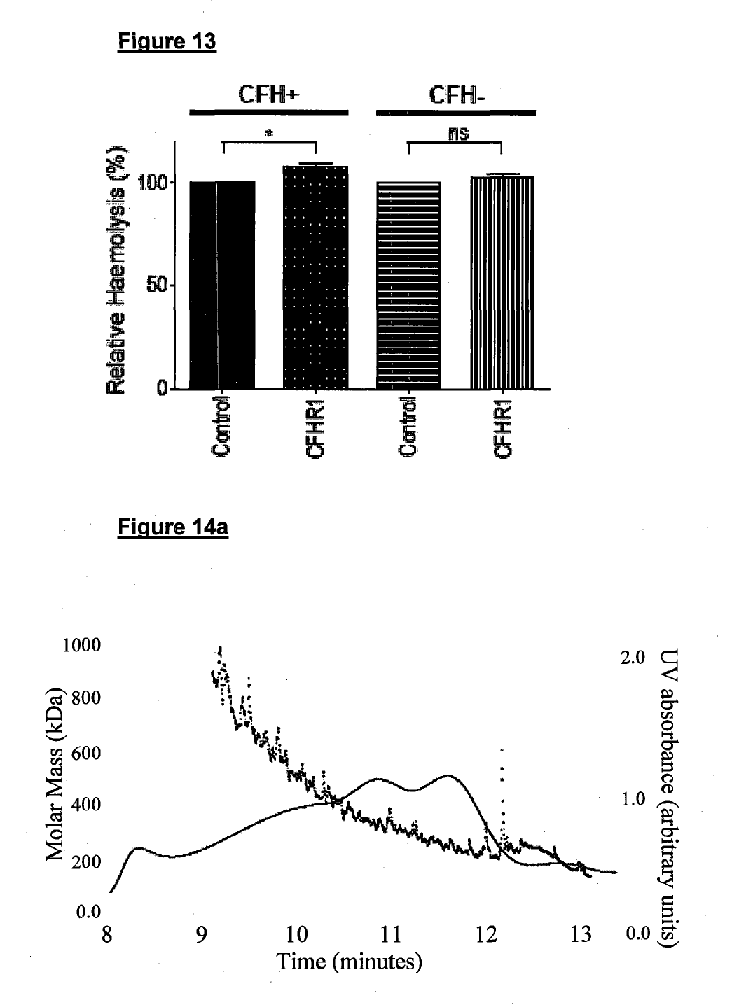

FIG. 13 shows that CFHR1 does not act as a complement regulator. Alternative pathway haemolysis assays were performed in a total volume of 200 μl containing 20% serum and approximately 106 guinea pig erythrocytes in 100 mM HEPES, 150 mM NaCl, 8 mM EGTA, 5 mM MgCl2, 0.1% gelatin, pH 7.5. Haemolysis was measured by the absorbance at 405 nm after 60 minutes at 370 C and appropriate control subtraction. Haemolysis using NHS and fH deficient serum was measured in the presence and absence of 700 nM CFHR1. All measurements were taken in triplicate and the control (no CFHR1 added) is taken as 100%. A significant increase in haemolysis was observed in factor H sufficient serum upon addition of CFHR1 (p=0.037);

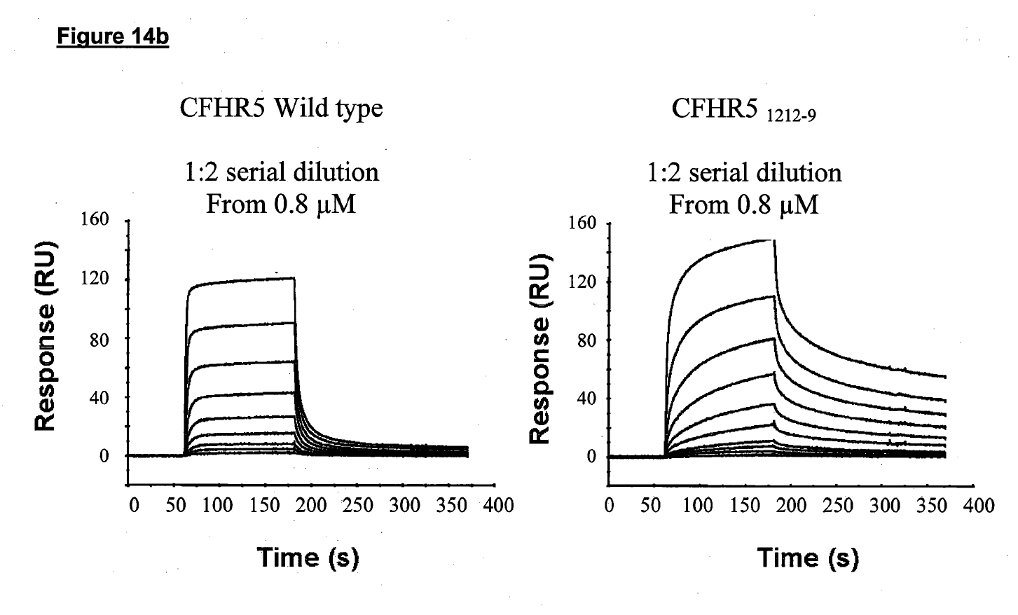

FIG. 14 shows the analysis of recombinant CFHR5₁₂₁₂₋₉. (a) Multi-angle light scattering analysis of CFHR5₁₂₁₂₋₉. Purified recombinant CFHR5₁₂₁₂₋₉ elutes as multiple species from an analytical gel filtration column with masses that range from approximately 130-950 kDa indicative of the formation of higher-order assemblies than dimers. (b) Comparison of CFHR5₁₂₁₂₋₉ versus CFHR5 wild-type binding to C3b coupled through the thioester. Dilutions of the proteins (from 0.8 μM) were flowed across immobilised C3b (408 RU amine-coupled C3b and 500 RU coupled through the thioester) and binding monitored. CFHR5₁₂₁₂₋₉ demonstrated a different binding kinetics compared to wild-type CFHR5;

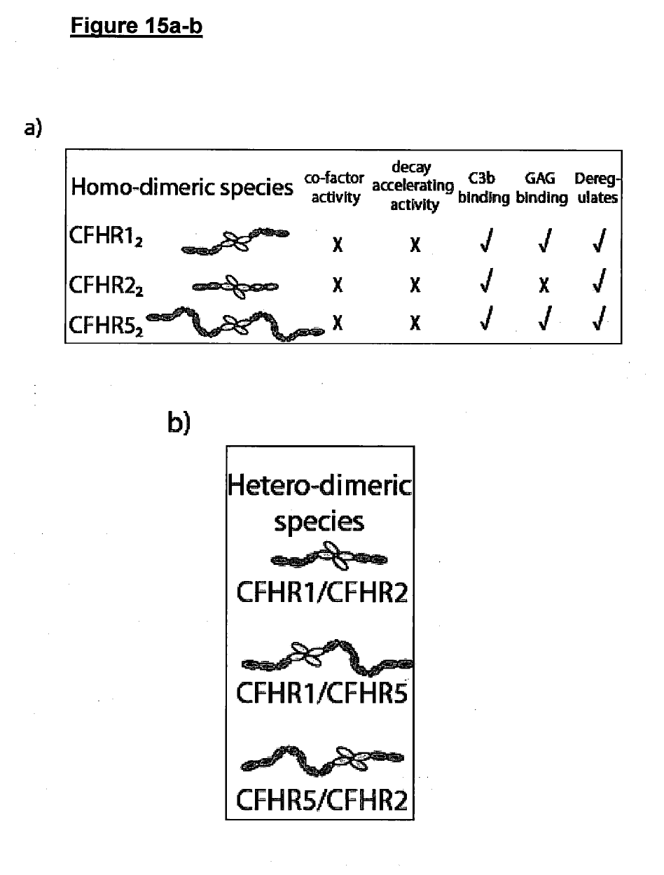

FIG. 15 shows a summary of the identities and activities of homodimeric species formed between CFHR1, CFHR2 and CFHR5. (a) A summary of the activities of each homo-dimeric species formed by CFHR1, CFHR2 and CFHR5 in serum. (b) Summary of the heterodimeric species formed between CFHR1, CFHR2 and CFHR5 which will have the properties of both components; and

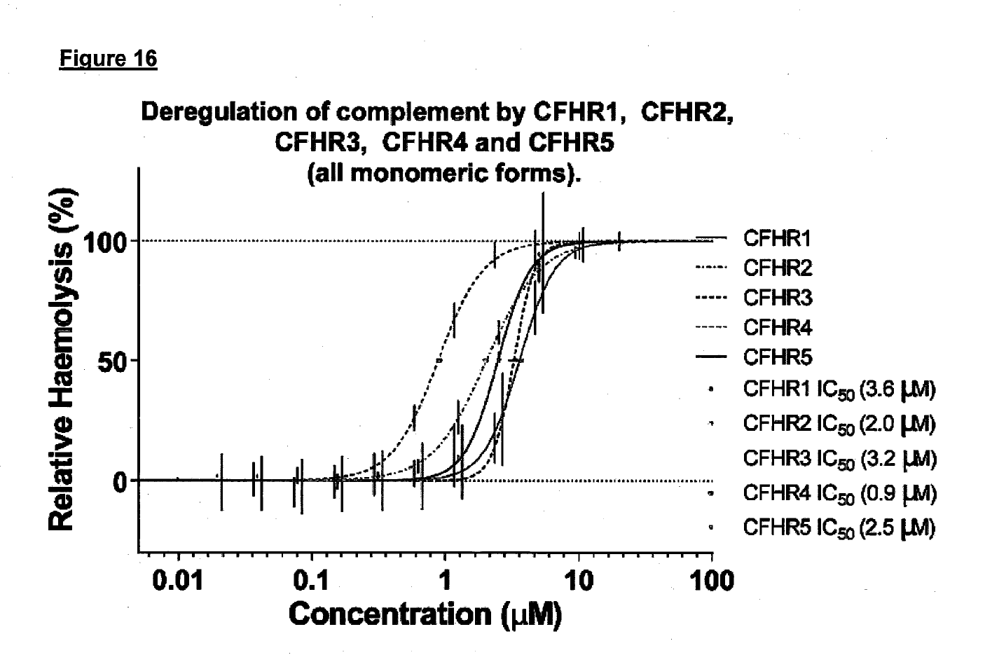

FIG. 16 shows deregulation of complement by CFHR1, CFHR2, CFHR3, CFHR4 and CFHR5 (all monomeric forms).

### MATERIALS AND METHODS

### Protein Expression and Purification

The gene encoding CFHR112 was amplified and inserted into the pKLAC2 vector using primers CFHR1₁_{_{—}} For [SEQ ID NO:14] and CFHR1₂_{_{—}} Rev [SEQ ID NO:15] prior to transformation into Kluyveromyces lactis and selection of successful integrants as per the manufacturers instructions (New England Biosciences).

Primers
CFHR1₁_For
[SEQ ID NO: 14]
5′-gctgacaaggatgatctcgagaaaagagaagcaacattttgtgattt
tcc-3′
CFHR1₂_Rev
[SEQ ID NO: 15]
5′-gccgcccatggacatctaagtggacctgcatttgg-3′
CFHR1₃_For
[SEQ ID NO: 16]
5′-gagatataccatgggcacttcctgtgtgaatccgcccacagtac-3′
CFHR1₅_Rev
[SEQ ID NO: 17]
5′-gccggatcctctatctttttgcacaagttggatactccagttt
ccc-3′
CFHR2₃_For
[SEQ ID NO: 18]
5′-tataccatgggcgaaaaatgtgggccccctccacctattgacaa
tgg-3′
CFHR2₄_Rev
[SEQ ID NO: 19]
5′-cgtgccggatcctatttttcttcacaactgggatataccagttt
ccc-3′
CFHR5₁_For
[SEQ ID NO: 20]
5′-caagttcctacaggggaagttttctcttactactgtgaagagaattt
tgtgtctccttcaaaatcct-3′
CFHR5₂_Rev
[SEQ ID NO: 21]
5′-aggattttgaaggagacacaaaattctcttcacagtagtaagagaaa
acttcccctgtaggaacttg-3′

K. lactis expressing CFHR112 was grown in a minimal media and the secreted target protein purified from the culture supernatant using size exclusion chromatography (Column; S75 16/60 (GE Healthcare) followed by ion exchange chromatography (Column; Mono Q 5/50 (GE Healthcare). Buffer A; 25 mM Tris, 10 mM NaCl, pH 7.5. Buffer B; 25 mM Tris, 1M NaCl, pH 7.5).

CFHR1345 and CFHR234 were amplified and inserted into the pET-15b vector (Novagen) using primers CFHR1₃_{_{—}} For [SEQ ID NO:16], CFHR1₅_{_{—}} Rev [SEQ ID NO:17], CFHR2₃_{_{—}} For [SEQ ID NO:18] and CFHR2₄_{_{—}} Rev [SEQ ID NO:19]. Both proteins were expressed in Escherichia coli strain BL21(DE3) and refolded from inclusion bodies based on the protocol by White et al with the substitution of the published refold buffer for 1 mM Cysteine, 2 mM Cystine, 20 mM Ethanolamine, 1 mM EDTA, pH 11.0. Refolded proteins were further purified using size exclusion chromatography (Column; 57516/60 (GE Healthcare). Buffer: 50 mM Tris, 150 mM NaCl, pH 7.5). Full-length CFHR5 cDNA was cloned into a modified version pCAGGS plasmid. CFHR5dimer mutant was generated by multi site-directed mutagenesis (Stratagene) according to manufacturer's instructions using primers CFHR5₁_{_{—}} For [SEQ ID NO:20] and CFHR52_Rev [SEQ ID NO:21]. Recombinant CFHR5 and CFHR5dimer mutant proteins were expressed in HEK293 cells. Recombinant proteins were purified by a single affinity chromatography step. Wild-type CFHR5 supernatant was applied onto a Hitrap NHS activated HP (GE Healthcare) column coated with MBC125 mouse monoclonal anti-CFHR1/2/5 antibody. CFHR5dimer mutant supernatant was applied onto a Hitrap NHS-activated HP column coated with rabbit anti-human CFHR5 antibody (a gift from Dr. J. McRae). After extensive washes with PBS and 0.5M NaCl-containing buffers, bound protein was eluted with 50 mM diethylamine and fractions were neutralized with 1/10 volume of 1M Tris pH7.

EDTA-plasma derived CFHR1, CFHR2 and CFHR5 used for haemolytic assays were co-purified using the Hitrap NHS-activated HP column coated with MBC125 mouse monoclonal anti-CF HR1/2/5 2 antibody following the same method as described above for recombinant CFHR5. Identical EDTA plasma volume was used for the purification for each sample. Native CFHR1, CFHR2 and CFHR5 used for MALS were co-purified using the Hitrap NHS-activated HP column coated with MBC125 mouse monoclonal anti-CFHR1/2/5 antibody as above but omitting the NaCl wash step. Following elution from MBC125 affinity column, protein was dialysed against 10 mM sodium phosphate pH7.8 and loaded onto a Mono Q column (GE Healthcare) in the same buffer. Protein was eluted using a gradient to 300 mM NaCl over 25 column volumes (CVs) and the major peak (eluting at approximately 120 mM NaCl) was used for subsequent analysis using MALS.

### Crystallisation and X-Ray Data Collection

Crystals were grown using the sitting drop vapour diffusion method from 0.2 μL, protein+0.2 μL mother liquor drops at 210 C using protein stocks at A280=3.3 and 7.8 for CFHR112 and CFHR234 respectively. CFHR112 crystals grew from a mother liquor containing 36% PEG 2000 MME, 0.1M MES pH 6.5. CFHR234 crystals grew in 30% PEG 8000, 0.2M ammonium sulphate. Crystals were plunge cooled in liquid nitrogen following cryoprotection in 20% and 15% ethylene glycol for CFHR112 and CFHR234, respectively. Data were collected at both the ESRF and DIAMOND using the rotation method with oscillation ranges of 0.150 or 0.20 at 120 K. CFHR112 data were collected at beamline ID29 (ESRF, Grenoble) with λ=1.7105 Å. CFHR234 data were collected at beamline I04-1 (DIAMOND, UK) with λ=0.9173 Å. Data were integrated and scaled using XIA2 19 with the −3dii option to enforce usage of XDS 20 for integration and SCALA for scaling 21.

### Structure Solution and Refinement

The structures of CFHR112 and CFHR234 were solved by molecular replacement using PHASER 22 with models derived from fH67 (PDB id: 2UWN) and fH19-20 (PDB id: 2G7I) respectively. Models were refined iteratively with manual rebuilding in COOT 23 and refinement using autoBUSTER 24. Data collection and refinement statistics are shown in Table 1. Ramachandran plots show that for CFHR112 93.4% of residues are in the favoured and 0.4% in the disallowed and for CFHR234 98.4% favoured, 0% disallowed.

### C3b Binding Competition Assay

C3b at 25 μg/ml in 0.1M NaHCO3 pH 9.5 buffer was immobilised in microtiter well plate (NUNC) overnight at 4° C. After blocking for 1 hour at room temperature with PBS containing 2% BSA, 0.073 μM of CFH alone or in combination with serial dilutions of CFHR5, CFHR1, CFHR1345 and CFHR234 (starting at 0.584 μM, 1.8 μM, 18 μM and 16 μM, respectively) were incubated for 2 hours at room temperature. A monoclonal anti-CFH (OX24) antibody was used as a detection antibody. Optical density (OD) values at 450 nm were corrected and expressed as a percentage of CFH binding considering 100% those OD values where CFH was incubated in the absence of CFHR proteins.

### Fluid Phase CFI Cofactor Activity Assays

CFH or soluble complement receptor 1 (sCR1) CFI cofactor activity for the cleavage of either C3b or iC3b was done as previously described 25. CFHR5 cofactor activity was tested under the same conditions.

### Detection of Heterodimers by Immunoprecipitation and ELISA Assays

Detection of heterodimers CFHR1-CFHR2 and CFHR1-CFHR5 were identified by immunoprecipitation. 50 μl of serum from an individual with 2 copies of the CFHR3-1 genes or from an individual lacking these genes (ΔCFHR3-1 homozygote) were diluted 1/10 in PBS and incubated with either a monoclonal anti-CFHR2 antibody (MBI-18) or with a monoclonal anti-CFHR5 (R&D Systems) antibody for 1 h at 4° C. In parallel, as a negative control for the immunoprecipitation, samples were not incubated with any antibody. Protein A/G sepharose beads previously washed with PBS were added and incubated overnight at 4° C. After extensive washes of the beads with PBS, bound proteins were eluted in protein loading buffer, separated using SDS-PAGE and analysed by western blotting using the anti-CFHR1/2/5 antibody (MBC125) followed by a HRP-conjugated rabbit anti-mouse IgG antibody (DAKO). Detection of heterodimer CFHR2-CFHR5 from serum was identified by enzyme-linked immunosorbent assay using rabbit anti-human CFHR5 (Abcam) and mouse anti-human CFHR2 (MBI-18) antibodies as capture and detection antibodies, respectively.

### Administration of CFHR5 to Cfh−/− Mice and Immunohistochemistry Studies

Cfh−/− mice were injected intravenously with 30 μg of either recombinant CFHR5 or CFHR5dimer mutant protein. Mice were sacrificed 2 hours post-injection and immunostaining performed on snap-frozen renal tissue performed as previously described 11. Mouse C3 was detected using a FITC-conjugated goat anti-mouse C3 antibody (MP Biomedicals, CA, USA). CFHR5 staining was performed using a polyclonal rabbit anti-human CFHR5 antibody (Abcam). Glomerular fluorescence intensity was calculated using image analysis software (Image-Pro Plus 7.0) and an Olympus U-TV1X-2 camera. We assessed 20 glomeruli from mice injected with identical concentration of either recombinant CFHR5 (n=2) or CFHR5dimer mutant protein (n=2). The median arbitrary fluorescence was significantly different between the two groups when calculated using either the total glomeruli counted in each group (n=40, p<0.05, unpaired t test) or when comparing per animal (n=2 per group, 20 glomeruli per animal, p<0.05, unpaired t test). The experiment was repeated with a separate batch of recombinant CFHR5 or CFHR5dimer mutant protein and glomerular binding of the CFHR5dimer mutant protein was again reduced.

### Haemolytic Assays

Alternative pathway haemolysis assays were performed in a total volume of 200 μl containing 20% serum and approximately 106 guinea pig erythrocytes in 100 mM HEPES, 150 mM NaCl, 8 mM EGTA, 5 mM MgCl2, 0.1% gelatin, pH 7.5. Haemolysis was measured by the absorbance at 405 nm after 60 minutes at 370 C and appropriate control subtraction. Dilution series of CFHR1, CFHR1345, CFHR234 and CFHR5, ranging from 1 nM to 9 μM, were added to reactions that had been supplemented with 140 nM CFH. All measurements were recorded in triplicate and are presented as haemolysis relative to the level of lysis in the absence of any CFHR proteins (o %) and 100% lysis by H2O. The effect of the CFHR5₁₂₁₂₋₉ mutation upon deregulation was assessed by comparison to the level of haemolysis by the wild type protein. CFHR1, CFHR2 and CFHR5 were co-purified from individuals with wild-type or mutant CFHR5 and haemolysis was measured using the same protocol described above with the addition of the CFHRs to reconstitute the serum levels in each individual. All measurements were performed in triplicate and are reported as percentages of maximum lysis by H2O. Haemolysis using normal human sera and CFH-deficient serum was measured in the presence and absence of 700 nM CFHR1 using the same protocol without the addition of CFH. All measurements were performed in triplicate and are reported as percentages of maximum lysis by H2O.

### Heparin Binding

Approximately 0.5 mg CFHR1345 and CFHR234 in 50 mM Tris, 10 mM NaCl, pH 7.5 was loaded onto a 1 ml HiTrap Heparin column (GE Healthcare) using an AKTAfplc (GE Healthcare). Non-bound material was washed out with 5 CVs 50 mM Tris, 10 mM NaCl, pH 7.5 prior to a gradient elution of 50% 50 mM Tris, 1M NaCl, pH 7.5 over 15 CVs. The conductivity at which the peak elutes was recorded for each sample.

### Multi Angle Laser Light Scattering

100 μg of sample was injected onto an S200 16/60 column (GE Healthcare. Buffer: 50 mM tris, 150 mM NaCl, pH 7.5) and the elution monitored using a Dawn Helios II (Wyatt Technology) and an Optilab TrEX (Wyatt Technology). All data and were analysed using ASTRA (Wyatt Technology).

### Surface Plasmon Resonance

All data in FIGS. 9-11 were gathered using a Biacore T100 (GE Healthcare). A reference channel that was mock activated-deactivated was included on each chip. For kinetic studies, samples were injected using the KINJECT command, in HBS/P (10 mM HEPES pH7.4, 150 mM NaCl, 0.05% surfactant-P20) flowed at 30 μl/min and analysed at 25° C. All kinetic data were double-referenced (data from reference cell and blank injection subtracted). The chip surface was regerated between cycles using 10 mM sodium acetate pH 4.0, 1 M NaCl. C3b (Comptech, Tyler, USA) was primary amine-coupled (deposition levels=150-400RU) to a CM5 chip following manufacturer's instructions (GE Healthcare). Where binding to clustered C3b was under investigation, further C3b was deposited by forming C3 convertase on amine-coupled C3b by flowing 100 μg/ml FB and 1 μg/ml factor D using the same buffer supplemented with 1 mM MgCl2, followed by C3 as substrate 26 resulting in 625RU of nascent C3b covalently bound to the chip surface. To generate iC3b, the surface was treated with 3 successive cycles of CFH (15.5 μg/ml) and factor I (10 μg/ml) until C3 convertase could no longer be formed. To generate C3dg, the iC3b surface was treated with soluble CR1 (gift from T Cell Sciences, 3 cycles at 5 μg/ml, 3 cycles at 50 μg/ml) and factor I (10 μg/ml). For kinetic analyses, CFH or CFHR5 were dialysed into HBS/P and each was flowed across the surface at a range of concentrations as indicated (1:2 serial dilution), with a regeneration step between each cycle. Data were analysed by steady state equilibrium analysis. Cofactor activity was assessed by flowing CFHR5 (0.18 μM and 0.44 μM over two 120 s cycles) with factor I (10 μg/ml) across the surface for 2 mins at 10 μl/min. As a positive control, CFH (0.1 μM) was flowed with factor I for 120 s. The capacity of C3b on the surface to form a convertase was assessed before and after CFH/CFHR5/factor I injection by flowing CFB and factor D, decrease in convertase formation indicated cleavage of C3b to iC3b. All data in FIG. 12 were collected on a Biacore 3000 instrument (GE Healthcare) using CM5 chips to which proteins were immobilised via standard primary amine coupling protocols. A reference channel that was mock activated-deactivated was included on each chip. HBS-EP buffer was used throughout. 2300 RU CFHR1, 750 RU CFHR112 and 1800 RU CFHR1345 were immobilized on a chip. 50 μl of 400 nM C3b (Calbiochem) was flowed over the surface at 20 μl/min using the KINJECT command with a dissociation time of 400 seconds. A dilution series of C5 (Calbiochem) between 50 nM and 400 nM was injected in an identical manner. All curves were reference subtracted and analysed using BIAEVALUATION (GE Healthcare).

### EXAMPLES

The complement system is a key component of the early, innate, immune system. Genetic variation in complement regulation influences susceptibility to age-related macular degeneration (AMD), meningitis and kidney disease. Variation includes genomic rearrangements within the complement factor H-related (CFHR) locus. Unfortunately, up until now, elucidating the mechanism underlying these associations has been hindered by the lack of understanding of the biological role of CFHR proteins. In the following examples, however, the inventors present unique structural data demonstrating that at least three of the CFHR proteins (CFHR1, 2 and 5) contain a shared dimerisation motif and that this hitherto unrecognised structural property enables formation of both homodimers and heterodimers. The examples also show that dimerisation confers avidity for tissue-bound complement fragments and enables these proteins to efficiently compete with the physiological complement inhibitor, complement factor H (CFH), for ligand binding. The data go on to demonstrate that these CFHR proteins function as competitive antagonists of CFH to modulate complement activation in vivo and explain why variation in the CFHRs predisposes to disease.

### Example 1

### CFHR1, CFHR2 and CFHR5 Contain a Novel Dimerization Motif

Comparing the amino acid conservation between CFHR1, CFHR2 and CFHR5 and CFH demonstrated that the CFHR proteins do not possess the residues implicated in the complement regulatory activity of CFH (cyan, FIG. 1a) but that these CFHRs shared a unique pair of highly conserved N-terminal domains (>85% sequence identity, FIG. 1a). The inventors therefore determined the crystal structure of the first two SCR domains of CFHR1 (CFHR1₁₂), which revealed that these domains assemble as a tight head-to-tail dimer with residues Tyr34, Ser36 and Tyr39 playing key roles in stabilising the assembly (FIG. 1b-d, Table 1).

TABLE 1
Data collection and refinement statistics

| | CFHR1₁ [symbol] | | CFHR2 [symbol] | |
|---|---|---|---|---|
| Data collection | | | | |
| Space group | P2 [symbol] 2 [symbol] 2 [symbol] | | P2 | |
| Cell dimensions | | | | |
| a, b, c (Å) | 45.3, 46.9, 111.7 | | 53.0, 25.2, 95.7 | |
| α, β, γ ( [symbol] ) | 90.0, 90.0, 90.0 | | 90.0, 93.8, 90.0 | |
| Resolution (Å) | 55.8-2.0 | (2.1-2.0) | 95.5-2.0 | (2.1-2.0) |
| R [symbol] | 0.09 | (0.54) | 0.05 | (0.26) |
| [symbol] σ [symbol] | 11.2 | (2.9) | 15.2 | (4.0) |
| Completeness (%) | 96.6 | (90.6) | 96.7 | (86.8) |
| Redundancy | 6.2 | (6.4) | 3.2 | (2.6) |
| Refinement | | | | |
| Resolution (Å) | 1.99-55.83 | (1.99-2.15) | 2.00-19.09 | (2.00-2.12) |
| No. reflections | 16261 | (1724) | 16963 | (2567) |
| R [symbol] R [symbol] | 0.22/0.25 | (0.22/0.26) | 0.21/0.24 | (0.21/0.27) |

| No. atoms | | |
|---|---|---|
| Protein | 1973 | 1952 |
| Ligand/ion | 166 | 117 |
| Water | 102 | 77 |
| B-factors (Å [symbol] ) | | |
| Protein | 52 | 27 |
| Ligand/ion | 53 | 40 |
| Water | 50 | 25 |
| R [symbol] diviations | | |
| Bond lengths (Å) | 0.008 | 0.010 |
| Bond angles ( [symbol] ) | 0.98 | 1.10 |

[symbol]

Highest resolution shell is shown in parenthesis. [symbol]
indicates data missing or illegible when filed

The recombinant CFHR1₁₂ fragment was also homogenously dimeric in solution (FIG. 2a) and the only conditions under which the chains can be separated is by reducing SDS-PAGE (FIG. 2a). Surprisingly, the dimer interface is highly conserved amongst CFHR1, CFHR2 and CFHR5 (FIGS. 1c and d). This conservation, together with the structural data, shows that CFHR1, CFHR2 and CFHR5 can assemble as hetero- as well as homo-dimers. The inventors next looked for the presence of these species in vivo.

### Example 2

### Plasma CFHR1, CFHR2 and CFHR5 Exist as Dimeric Species In Vivo

The inventors purified CFHR1, CFHR2 and CFHR5 from serum using a monoclonal antibody (MBC125; anti-CFHR1/2/5) that recognizes a shared epitope within the first two SCR domains of these proteins. When this purified preparation was analysed in solution by multi-angle laser light scattering (FIG. 2a) the observed mass range was 65-80 kDa. The lowest observed mass exceeded the predicted molecular mass of the smallest protein (CFHR2, predicted Mr=30 kDa), whilst the largest observed mass exceeded that of the largest protein (CFHR5, predicted Mr=64 kDa). This demonstrated that CFHR2 is not monomeric in vivo and was consistent with CFHR1, CFHR2 and CFHR5 dimerisation.

To look for heterodimers in vivo the inventors performed serum immunoprecipitation using either a specific anti-CFHR2 (MBC22; FIG. 2b) or anti-CFHR5 (FIG. 2c) antibody. In both assays, sera from individuals with and without the ΔCFHR3-1 deletion polymorphism were used and probed with the anti-CFHR1/2/5 antibody. This revealed the presence of CFHR1-CFHR2 (FIG. 2b) and CFHR1-CFHR5 (FIG. 2c) heterodimers in serum. The specificity of these assays was supported by the lack of these heterodimers in sera from individuals with the ΔCFHR3-1 deletion polymorphism. Detection of CFHR2-5 heterodimers using these assays was not possible because of the presence of non-specific bands in the region of CFHR5 (FIG. 2b) and CFHR2 (FIG. 2c). The inventors therefore designed an ELISA assay using anti-CFHR5 as a capture antibody and anti-CFHR2 as a detection antibody (FIG. 2d). This showed a strong signal using sera from two individuals homozygous for the ΔCFHR3-1 deletion whilst a weak or absent signal resulted when sera from individuals without this polymorphism was used. This demonstrated that the relative abundance of CFHR1, CFHR2 and CFHR5 influences the pattern of dimers present in vivo.

### Example 3

### Dimerisation Enhances the Interaction of CFHRs with Renal-Bound Mouse Complement C3 In Vivo

The inventors next explored the functional consequences of dimerisation. They predicted that dimerisation would enhance ligand interaction through avidity. To test this they generated monomeric and dimeric CFHR5 proteins. Monomeric CFHR5 (CFHR5^{dimer mutant}) was generated in vitro by mutating the three key amino acids within the dimerisation motif to the corresponding amino acids within CFH (Tyr34Ser, Ser36Tyr, Tyr39Glu, FIGS. 3a and b). CFHR5^{dimer mutant} was demonstrated to be monomeric using MALS (FIG. 3c). Next they examined the interaction of monomeric and dimeric CFHR5 with tissue-bound complement in a mouse model. Gene-targeted CFH-deficient mice have florid deposition of activated mouse C3 along the glomerular basement membrane (GBM) within the kidney. Human CFHR5 was able to interact with the GBM-bound C3 in a specific and dose-dependent manner (FIG. 8). Using this model the interaction of intravenously administered monomeric CFHR5 with GBM-bound mouse C3 was significantly reduced compared to that of the dimeric protein (median glomerular staining=227 and 95 arbitrary fluorescence units, for wild-type and dimer mutant respectively, P<0.05, unpaired t test, FIG. 3d). This indicated that dimerisation of CFHR5 enhanced its ability to interact with mouse C3 in vivo.

### Example 4

### Dimerisation Enhances the Ability of CFHR1 and CFHR5 to Compete with CFH for C313 Binding In Vitro

The inventors next speculated that dimerisation of CFHR1, CFHR2 and CFHR5 would enable these proteins to efficiently compete with CFH for interaction with C3 in vivo. Since CFH, CFHR1 and CFHR5 contain the same carboxyl-terminal C3b/C3d binding site (FIG. 1a, FIG. 6), the inventors developed an ELISA assay to determine if CFHR1 and CFHR5 influence the interaction of CFH with C3b. This demonstrated that the CFH-C3b interaction was inhibited in a dose dependent manner at physiologically relevant concentrations by native dimers of CFHR5 (dose range 0.005 to 0.6 μM) and CFHR1 (dose range 0.014 to 1.8 μM) (FIG. 4a). Monomeric constructs of CFHR1 and CFHR2 that lack the dimerization domains (denoted CFHR1₃₄₅ and CFHR2₃₄, respectively) could also inhibit CFH binding but at higher concentrations (FIG. 4a).

### Example 5

### CFHR1 and CFHR5 De-Regulate Complement Activation by Acting as Competitive Antagonists of CFH

To determine the physiological relevance of the competition between CFHR1/CFHR5 and CFH for C3b binding the inventors have studied the ability of CFHR1 and CFHR5 to regulate C3. Using surface plasmon resonance (SPR), in which the sensor surface was coated with either amine or thioester coupled C3b (monomeric or ‘clustered’ C3b respectively; FIG. 9), or thioester-coupled iC3b and C3dg (FIG. 10), CFHR5 bound to C3b, iC3b and C3dg but there was no evidence of fluid-phase factor I cofactor activity (figure ii). CFHR1 has previously been reported to inhibit the C5 not C3 convertase by binding to C5/C5b6 but the inventors were unable to detect any significant interaction with C5 (FIG. 12). Moreover, they were unable to detect any evidence of complement regulatory activity when CFHR1 was investigated in alternative pathway haemolysis assays (FIG. 13). These data indicated that CFHR1 and CFHR5 have no intrinsic C3 or C5 regulatory activity at physiological concentrations. They therefore hypothesized that these proteins, through their ability to compete with CFH for binding to C3b, actually prevent CFH-mediated complement regulation.

To test this, the inventors utilized a complement-dependent haemolytic assay comprising unopsonised guinea-pig erythrocytes (a complement activating surface) incubated with 20% normal human sera. The addition of 100 nM CFH resulted in 50% inhibition of cell lysis and therefore enabled us to determine if exogenous CFHR proteins increased or decreased haemolysis. Using these conditions, in which the total CFH concentration in the assay was approximately 0.5 μM (100 nM added to assay in addition to 20% normal human sera), they added increasing concentrations of concentrations of CFHR1₃₄₅, CFHR2₃₄, serum-derived CFHR1 and recombinant CFHR5 (FIG. 4b). Surprisingly, these preparations increased rather than decreased haemolysis in a dose-dependent fashion. Importantly, the IC50 was significantly lower for the dimeric CFHR1 (0.7 μM) and CFHR5 (0.15 μM) compared to the monomeric CFHR1 (3.6 μM) and CFHR2 (4.7 μM) fragments. These data demonstrated that CFHR1 and CFHR5 can interfere with the C3b inhibitory actions of CFH by acting as competitive antagonists and that this interference is enhanced by dimerisation. The inventors refer to this process as complement de-regulation because it emphasizes the point that these proteins have no ability to influence complement regulation in the absence of CFH.

### Example 6

### De-Regulation b CFH Mutation Associated with Familial C3 Glomerulopathy

In patients with familial complement-mediated kidney disease, termed C3 glomerulopathy, there is a heterozygous CFHR5 mutation in which the initial two N-terminal domains are duplicated. The data presented here reveal that this results in duplication of the dimerisation motif (denoted CFHR 5₁₂₁₂₋₉). When they generated recombinant CFHR5₁₂₁₂₋₉ it was clear that the purified preparation readily ‘aggregated’ and was associated with atypical C3 binding kinetics using SPR (FIG. 14). When they elucidated the dimerisation domain, they re-interpreted this aggregation as a direct consequence of duplicated dimerisation domains (enabling multimeric interaction) rather than an in vitro artefact. A further consequence of the structural data was that examination of the isolated recombinant CFHR5₁₂₁₂₋₉ was irrelevant pathophysiologically since it was likely that CFHR5₁₂₁₂₋₉ interacted with CFHR1, CFHR2 and the wild-type CFHR5 (derived from the unaffected allele) in vivo. Consequently, they tested whether de-regulation is influenced in these patients by comparing plasma preparations containing all CFHR1, CFHR2 and CFHR5 species from affected individuals and healthy controls without the CFHR3-1 deletion polymorphism (FIG. 4c). This showed that patient preparations resulted in significantly greater haemolysis than that of controls.

discussion

The data presented herein provide compelling evidence that CFHR1, CFHR2 and CFHR5 at physiologically relevant concentrations interfere with the complement inhibitory activities of CFH. This process, which the inventors term de-regulation, is influenced by the ability of these proteins to form dimers (FIG. 5). This structural property confers avidity enabling these dimeric molecules to compete with CFH for ligand due to the fact that the C-terminal C3b/C3d recognition sites are essentially conserved between the CFHR proteins and CFH. The shared dimerisation domain between CFHR1, CFHR2 and CFHR5 enabled the formation of both homo- and heterodimers. The dimerisation motif that has been characterized is not present within CFHR3 and CFHR4 but it has been suggested that CFHR4, at least (and possibly also CFHR3), may also exist as a dimer. Accordingly, CFHR3 and CFHR4 are also believed to form dimers and behave as competitive antagonists of CFH.

The inventors were able to demonstrate heterodimers within CFHR1, CFHR2 and CFHR5 and the specificity of these interactions was evident when comparing sera from individuals with and without CFHR1. A priori the inventors predicted that homo and heterodimers containing CFHR1 would predominate in sera from individuals without the ΔCFHR3-1 deletion polymorphism since this protein is most abundant with a mean serum concentration equimolar to that of CFH (CFH=116-562 μg/ml, 0.7-3.6 μM, mean 2.1 μM (13), CFHR1=70-100 μg/ml, 1.7-2.5 μM, mean 2.1 μM (11)). In contrast the median concentration of CFHR5 (3-6 μg/ml, 0.05-0.09 μM, mean 0.07 μM (14)) is much lower. The inventors are not aware of published estimates for the circulating concentration of CFHR2 but the data suggest its concentration is intermediate between CFHR1 and CFHR5 (Coomassie gel inset, FIG. 2a). Consistent with the predominance of CFHR1-containing dimers, CFHR2-CFHR5 heterodimers were only readily detectable in sera from patients deficient in CFHR1 (those with the ΔCFHR3-1 deletion polymorphism).

The inventors were unable to demonstrate C3 regulatory activity for CFHR5 and were unable to demonstrate an interaction between CFHR1 and C5. Interestingly, although CFHR3 has previously been reported as a regulator of complement (in non-physiological conditions), other experiments reported in the same paper demonstrate that, as shown here for CFHR1, CFHR2 and CFHR5, CFHR3 can also de-regulate CFH. Recently, CFHR4 was shown to be devoid of intrinsic complement activity but able to act as a platform on which complement activation could proceed unhindered. Therefore, if CFHR4 was able to compete for CFH ligands then it too has the potential to de-regulate CFH activity. Taken together, the data suggest that the CFHR1, CFHR2 and CFHR5 modulate complement activation by competing with CFH for C3b binding. In contrast to CFH-C3b interaction which prevents further C3b generation (negative regulation), the interaction of these CFHR proteins with C3b enables C3b amplification to proceed unhindered. The ability of CFHR proteins to de-regulate CFH would be predicted to be influenced by many factors including (1) the concentration and composition of the CFHR proteins relative to CFH in the vicinity of complement activation, (2) the spatial density of deposited C3 (for example, they speculate that the action of large dimers such as CFHR5-CFHR5 may be important when spatial density is low), (3) the polyanion composition of the surface upon which complement is activated since the polyanion affinities of the different CFHR proteins may vary and (4) the flow rate across the site of complement activation in surfaces in contact with blood (the enhanced avidity of dimeric species would favour their interaction with ligand relative to CFH under high flow) such as within the kidney.

The data had obvious implications for how one considers the impact of the C3 glomerulopathy-associated CFHR5 mutation in which there is duplication of the dimerisation domain (duplication of SCR1 and SCR2, CFHR5₁₂₁₂₋₉)(8). Theoretically, this duplication would result in trimeric or higher order complexes. However, since CFHR1 is abundant in vivo, the inventors speculate that the most common species would be trimeric and composed of two molecules of CFHR1 complexed with CFHR5₁₂₁₂₋₉. When they purified CFHR1, CFHR2, CFHR5 and CFHR5₁₂₁₂₋₉ from an affected individual, this serum fraction was more potent in de-regulation than serum fractions from healthy controls. If it is assumed that CFH plays a physiological role in protecting the GBM from C3 activation, the data would suggest that C3 glomerulopathy develops in individuals since the presence of CFHR5₁₂₁₂₋₉ results in a greater degree of CFHR-mediated de-regulation.

CFH serum levels are not actively regulated in an individual, varying only under extreme conditions such as meningococcal sepsis where tight interactions with the bacterium deplete CFH. The inventors believe that fine-tuning of complement activation (complement modulation) can be achieved by altering CFHR levels. It is notable that in otitis media with effusion, where complement is strongly activated in the middle ear effusion fluid, CFHR5 levels were noted to be high and it was proposed that competition between CFHR5 and CFH might be relevant in this circumstance. This requires further study but the data presented here would predict that a local increase in CFHR protein concentration would, through enhanced CFH de-regulation, enable rapid enhancement of complement activation. The opposite might be achieved by down-regulating CFHR concentrations thereby reducing de-regulation.

In summary, the inventors clearly show that these proteins can bind bivalently to adjacent molecules of C3b (or iC3b/C3dg/C3d) deposited on the membrane, and that these dimers are not artifacts of expression in P. pastoris, but occur in the plasma. In addition, the inventor have demonstrated, using surface Plasmon resonance (SPR), that CFHR5 (that has several modules between its dimerisation site and its C3b-binding site) binds surprisingly well to clustered C3b molecules, but not so well to spaced-apart C3b molecules, and this may suggest that CFHR1-5 are sensitive to the distribution of C3b molecules, and can therefore modulate the regulatory activity of CFH accordingly. These observations have revealed an exciting and novel function of the CFHR proteins. The inventors propose that these molecules have evolved to enable complement to be modulated at a sophisticated level under diverse circumstances. Understanding how these proteins modulate activation during infection, tissue injury and inflammation will enable us not only to gain further understanding of the role of complement in disease but also to devise novel strategies to increase or decrease complement activation therapeutically.

### Example 7

### CFHR1, CFHR2, CFHR3, CFHR4 and CFHR5 De-Regulate Complement Activation by Acting as Competitive Antagonists of CFH

In Example 5, the inventors have already shown that CFHR1 and CFHR5, through their ability to compete with CFH for binding to C3b, prevent CFH-mediated complement regulation. The inventors then set out to test CFHR3 and CFHR4, using a complement-dependent haemolytic assay comprising unopsonised guinea-pig erythrocytes (a complement activating surface) incubated with 20% normal human sera (Goicoechea de Jorge et al., Dimerization of complement factor H-related proteins modulates complement activation in vivo. Proc Natl Acad Sci USA. 2013 Mar. 19; no (12):4685-90). The addition of 100 nM CFH resulted in 50% inhibition of cell lysis and therefore enabled them to determine if exogenous CFHR proteins increased or decreased haemolysis. Using these conditions, in which the total CFH concentration in the assay was approximately 0.5 μM (100 nM added to assay in addition to 20% normal human sera), they added increasing concentrations of concentrations of CFHR1, CFHR2, CFHR3, CFHR4 and CFHR5. The results are shown in FIG. 16.

Surprisingly, these preparations increased rather than decreased haemolysis in a dose-dependent fashion. Importantly, the IC5O are within the physiological range of these proteins. Accordingly, these data show that CFHR3 and CFHR4 de-regulate, and so validates the hypothesis that deregulation applies to all five of the CFHR proteins.

### REFERENCES

1. Pickering M C & Cook HT (2008) Translational mini-review series on complement factor H: renal diseases associated with complement factor H: novel insights from humans and animals. Clin Exp Immunol 151(2):210-230.
2. de Cordoba S R, Tortajada A, Harris C L, & Morgan B P (2012) Complement dysregulation and disease: From genes and proteins to diagnostics and drugs. Immunobiology 217(11):1034-1046.
3. Hageman G S, et al. (2006) Extended haplotypes in the complement factor H (CFH) and CFH-related (CFHR) family of genes protect against age-related macular degeneration: characterization, ethnic distribution and evolutionary implications. Ann Med 38(8):592-604.
4. Abarrategui-Garrido C, Martinez-Barricarte R, Lopez-Trascasa M, de Cordoba S R, & Sanchez-Corral P (2009) Characterization of complement factor H-related (CFHR) proteins in plasma reveals novel genetic variations of CFHR1 associated with atypical hemolytic uremic syndrome. Blood 114(19):4261-4271.
5. Gharavi A G, et al. (2011) Genome-wide association study identifies susceptibility loci for IgA nephropathy. Nat Genet 43(4):321-327.
6. Hughes A E, et al. (2006) A common CFH haplotype, with deletion of CFHR1 and CFHR3, is associated with lower risk of age-related macular degeneration. Nat Genet 38(10):1173-1177.
7. Zhao J, et al. (2011) Association of genetic variants in complement factor H and factor H-related genes with systemic lupus erythematosus susceptibility. PLoS Genet 7(5):e1002079.
8. Gale D P, et al. (2010) Identification of a mutation in complement factor H-related protein 5 in patients of Cypriot origin with glomerulonephritis. Lancet 376(9743):794-801.
9. Malik T H, et al. (2012) A Hybrid CFHR3-1 Gene Causes Familial C3 Glomerulopathy. J Am Soc Nephrol.
10. Pickering M C, et al. (2002) Uncontrolled C3 activation causes membranoproliferative glomerulonephritis in mice deficient in complement factor H. Nat Genet 31(4)424-428.
11. Heinen S, et al. (2009) Factor H-related protein 1 (CFHR-1) inhibits complement C5 convertase activity and terminal complex formation. Blood 114(12):2439-2447.
12. Hellwage J, et al. (1999) Functional properties of complement factor H-related proteins FHR-3 and FHR-4: binding to the C3d region of C3b and differential regulation by heparin. FEBS letters 462 (3)345-352.
13. Esparza-Gordillo J, et al. (2004) Genetic and environmental factors influencing the human factor H plasma levels. Immunogenetics 56 (2):77-82.
14. McRae J L, et al. (2005) Human factor H-related protein 5 has cofactor activity, inhibits C3 convertase activity, binds heparin and C-reactive protein, and associates with lipoprotein. J Immunol 174(10): 6250-6256.
15. Fritsche L G, et al. (2010) An imbalance of human complement regulatory proteins CFHR1, CFHR3 and factor H influences risk for age-related macular degeneration (AMD). Hum Mol Genet 19(23)4694-4704.
16. Hebecker M & Jozsi M (2012) Factor H-related Protein 4 Activates Complement by Serving as a Platform for the Assembly of Alternative Pathway C3 Convertase via Its Interaction with C3b Protein. J Biol Chem 287(23):19528-19536.
17. Narkio-Makela M, Hellwage J, Tahkokallio O, & Meri S (2001) Complement-regulator factor H and related proteins in otitis media with effusion. Clin Immunol 100(1):118-126.

1. An agent, which:—
(i) reduces the concentration or activity of at least one complement factor H-related (CFHR) protein selected from a group consisting of: CFHR1, CFHR2, CFHR3, CFHR4 and CFHR5; or
(ii) reduces or inhibits dimerisation or higher order assembly of at least one CFHR protein selected from a group consisting of: CFHR1, CFHR2, CFHR3, CFHR4 and CFHR5, for use in diagnosis or therapy.

2. An agent, which:—
(i) reduces the concentration or activity of at least one complement factor H-related (CFHR) protein selected from a group consisting of: CFHR1, CFHR2, CFHR3, CFHR4 and CFHR5; or
(ii) reduces or inhibits dimerisation or higher order assembly of at least one CFHR protein selected from a group consisting of: CFHR1, CFHR2, CFHR3, CFHR4 and CFHR5, for use in the treatment, prevention or amelioration of a disease characterised by excessive complement activation.

3. An agent according to claim 1, wherein the agent is used to treat, prevent or ameliorate meningitis, renal disease, including C3 glomerulopathy, autoimmune disease or inflammation including conditions, such as rheumatoid arthritis, asthma, lupus nephritis, ischemia-reperfusion injury, atypical haemolytic uremic syndrome, thrombotic thrombocytopenic purpura, paroxysmal nocturnal haemoglobinuria, Membranoproliferative glomerulonephritis, hemolytic uremic syndrome, Hypocomplementemic glomerulonephritis, dense deposit disease, macular degeneration (e.g. age-related macular degeneration, AMD), spontaneous foetal loss, Pauci-immune vasculitis, epidermolysis bullosa, recurrent foetal loss, multiple sclerosis, traumatic brain injury, Degos' disease, myasthenia gravis, cold agglutinin disease, dermatomyositis, Graves' disease, Hashimoto's thyroiditis, type I diabetes, psoriasis, pemphigus, autoimmune hemolytic anaemia, idiopathic thrombocytopenic purpura, Goodpasture syndrome, antiphospholipid syndrome, Infective endocarditis, or injury resulting from myocardial infarction, cardiopulmonary bypass and hemodialysis.

4. An agent according to claim 1, wherein the agent reduces the concentration or activity of, or reduces or inhibits dimerisation or higher order assembly of, at least one CFHR protein comprising an amino acid sequence substantially as set out in SEQ ID NO:2, 4, 6, 8, 9 or 11, or a functional variant or fragment thereof.

5. An agent according to claim 1, wherein the agent reduces the concentration or activity of, or reduces or inhibits dimerisation or higher order assembly of, at least one CFHR protein encoded by a nucleic acid sequence substantially as set out in SEQ ID No: 1, 3, 5, 7 or 10, or a functional variant or fragment thereof.

6. An agent according to claim 1, wherein the agent binds to domain 1 and 2 of any of SEQ ID NO:2, 4, 6, 8, 9 or 11, or a fragment of variant thereof, and thereby reduces the concentration or activity of, or reduces or inhibits dimerisation or higher order assembly of, the at least one CFHR protein.

7. An agent according to claim 1, wherein the agent binds to SEQ ID No.12 or SEQ ID No.27, or a fragment or variant thereof, and thereby reduces the concentration or activity of, or reduces or inhibits dimerisation or higher order assembly of, the at least one CFHR protein.

8. An agent according to claim 1, wherein the agent binds to SEQ ID No.13, or a fragment or variant thereof, and thereby reduces the concentration or activity of, or reduces or inhibits dimerisation or higher order assembly of, the at least one CFHR protein.

9. An agent according to claim 1, wherein the agent binds to a region of SEQ ID No.12, or a fragment or variant thereof, other than that which is represented by SEQ ID No.13, and thereby reduces the concentration or activity of, or reduces or inhibits dimerisation or higher order assembly of, the at least one CFHR protein.

10. An agent according to claim 1, wherein the agent reduces the concentration or activity of a dimer of the CFHR, wherein the dimer is either: (i) a homodimer selected from a group consisting of: CFHR1-CFHR1, CFHR2-CFHR2, CFHR3-CFHR3, CFHR4-CFHR4 and CFHR5-CFHR5, or (ii) a heterodimer selected from a group consisting of: CFHR1-CFHR2, CFHR1-CFHR3, CFHR1-CFHR4, CFHR1-CFHR5, CFHR2-CFHR3, CFHR2-CFHR4, CFHR2-CFHR5, CFHR3-CFHR4, CFHR3-CFHR5 and CFHR4-CFHR5.

11. An agent according to claim 1, wherein the agent:—
(a) reduces binding between a CFHR and a C3 fragment;
(b) increases binding between CFH and a C3 fragment;
(c) binds to a CFHR to reduce its biological activity; or
(d) decreases expression of a CFHR.

12. An agent according to claim 1, wherein the agent comprises a competitive polypeptide or a peptide-like molecule, or a derivative or analogue thereof; an antibody or antigen-binding fragment or derivative thereof; an aptamer (nucleic acid or peptide); a peptide-binding partner; or a small molecule that binds specifically to the CFHR protein to prevent it binding to a C3 fragment.

13. An agent according to claim 1, wherein the agent comprises a gene-silencing molecule.

14. An agent according to claim 1, wherein the agent comprises an antibody, or antigen binding fragment thereof.

15. An agent according to claim 14, wherein the antibody or antigen binding fragment thereof is raised against any of SEQ ID NO:2, 4, 6, 8, 9 or 11, or a fragment of variant thereof, acting as antigen.

16. An agent according to claim 14, wherein the antibody or antigen binding fragment thereof is raised against domains 1 and 2 of any of SEQ ID NO:2, 4, 6, 8, 9 or 11, or a fragment of variant thereof, acting as antigen.

17. An agent according to claim 14, wherein the antibody or antigen binding fragment thereof is raised against SEQ ID No.12, SEQ ID No.13 or SEQ ID No.27, acting as antigen.

18. An agent according to claim 14, wherein the antibody is known as “2C6”.

19. An antibody or antigen binding fragment thereof, which binds specifically to SEQ ID No.12 or SEQ ID No. 27, or a fragment or variant thereof.

20. An antibody or antigen binding fragment thereof according to claim 19, which binds specifically to SEQ ID No.13, or a fragment or variant thereof.

21. An antibody or antigen binding fragment thereof according to claim 19, which binds specifically to a region of SEQ ID No.12, or a fragment or variant thereof, other than that which is represented by SEQ ID No.13.

22. An antibody or antigen binding fragment according to claim 19, for use in reducing the concentration or activity of, or reducing or inhibiting dimerisation or higher order assembly of, at least one CFHR protein selected from a group consisting of: CFHR1, CFHR2, CFHR3, CFHR4 and CFHR5

23. An antibody or antigen binding fragment according to claim 19, for use in the treatment, prevention or amelioration of a disease characterised by excessive complement activation.

24. Use of SEQ ID NO:12 or SEQ ID No.13 or SEQ ID No.27, or a functional variant or fragment thereof, as an epitope for generating an antibody, or a functional fragment thereof.

25. Use according to claim 24, wherein SEQ ID NO:13 is used as an epitope for producing the antibody or functional fragment thereof.

26. A complement factor H-related (CFHR) gene-silencing molecule, for use in the treatment, amelioration or prevention of a disease characterised by excessive complement activation.

27. A gene-silencing molecule according to claim 26, wherein the molecule reduces expression of at least one CFHR protein selected from a group consisting of: CFHR1, CFHR2, CFHR3, CFHR4 and CFHR5.

28. A method for identifying an agent that modulates dimerisation or higher order assembly of at least one complement factor H-related (CFHR) protein selected from a group consisting of: CFHR1, CFHR2, CFHR3, CFHR4 and CFHR5, the method comprising:—
(i) contacting, in the presence of a test agent, a first protein selected from a group consisting of: CFHR1, CFHR2, CFHR3, CFHR4 and CFHR5, with a second protein selected from a group consisting of: CFHR1, CFHR2, CFHR3, CFHR4 and CFHR5; and
(ii) detecting binding between the first and second proteins, wherein an alteration in binding as compared to a control is an indicator that the agent modulates dimerisation or higher order assembly of at least one complement factor H-related (CFHR) protein selected from a group consisting of: CFHR1, CFHR2, CFHR3, CFHR4 and CFHR5.

29. A method for identifying a candidate agent, for use in the treatment, prevention or amelioration of a disease characterised by inappropriate complement activation, the method comprising:—
(i) contacting, in the presence of a test agent, a first protein selected from a group consisting of: CFHR1, CFHR2, CFHR3, CFHR4 and CFHR5, with a second protein selected from a group consisting of: CFHR1, CFHR2, CFHR3, CFHR4 and CFHR5; and
(ii) detecting binding between the first and second proteins, wherein an alteration in binding as compared to a control is an indicator that the agent is a candidate for the treatment, prevention of amelioration a disease characterised by inappropriate complement activation.

30. An assay for identifying an agent that modulates dimerisation or higher order assembly of at least one complement factor H-related (CFHR) protein selected from a group consisting of: CFHR1, CFHR2, CFHR3, CFHR4 and CFHR5, the method comprising:—
(i) a first protein selected from a group consisting of: CFHR1, CFHR2, CFHR3, CFHR4 and CFHR5;
(ii) a second protein selected from a group consisting of: CFHR1, CFHR2, CFHR3, CFHR4 and CFHR5; and
(iii) a vessel configured to permit contacting of at least one test agent with the first and/or second agent.

31. The ex vivo use of a colourimetrically- or fluorescentally-labelled first protein selected from a group consisting of: CFHR1, CFHR2, CFHR3, CFHR4 and CFHR5, and/or a colourimetrically- or fluorescentally-labelled second protein selected from a group consisting of: CFHR1, CFHR2, CFHR3, CFHR4 and CFHR5, for identifying an agent which modulates dimerisation or higher order assembly of at least one complement factor H-related (CFHR) protein selected from a group consisting of: CFHR1, CFHR2, CFHR3, CFHR4 and CFHR5.

32. A pharmaceutical composition, comprising an agent which:
(i) reduces the concentration or activity of at least one complement factor H-related (CFHR) protein selected from a group consisting of: CFHR1, CFHR2, CFHR3, CFHR4 and CFHR5; or
(ii) reduces or inhibits dimerisation or higher order assembly of at least one CFHR protein selected from a group consisting of: CFHR1, CFHR2, CFHR3, CFHR4 and CFHR5, and a pharmaceutically acceptable vehicle.

33. A pharmaceutical composition according to claim 32, wherein the agent comprises an antibody or antigen binding fragment thereof.

34. A process for making the pharmaceutical composition according to claim 32, the process comprising contacting a therapeutically effective amount of an agent which:
(i) reduces the concentration or activity of at least one complement factor H-related (CFHR) protein selected from a group consisting of: CFHR1, CFHR2, CFHR3, CFHR4 and CFHR5; or
(ii) reduces or inhibits dimerisation or higher order assembly of at least one CFHR protein selected from a group consisting of: CFHR1, CFHR2, CFHR3, CFHR4 and CFHR5, and a pharmaceutically acceptable vehicle.
